(19) United States
(12) Patent Application Publication
(10) Pub. No.: US 2026/0201470 A1
(43) Pub. Date: Jul. 16, 2026

(54) CIRCULATING PERIPHERAL BLOOD MONOCYTES AS A PROGNOSTIC MARKER FOR COMPLICATED AND RESISTANT CROHN'S DISEASE

(71) Applicant: CEDARS-SINAI MEDICAL CENTER, Los Angeles, CA (US)

(72) Inventors: Rebecca GONSKY, Los Angeles, CA (US); Stephan R. TARGAN, Santa Monica, CA (US)

(21) Appl. No.: 19/132,743

(22) PCT Filed: Nov. 27, 2023
(86) PCT No.: PCT/US2023/081196

§ 371 Date: May 23, 2025

Related U.S. Application Data
(60) Provisional application No. 63/428,362, filed on Nov. 28, 2022.
(60) Provisional application No. 63/500,211, filed on May 4, 2023.

(51) Int. Cl. C12Q 1/6883; G16B 25/00

(52) U.S. Cl. CPC C12Q 1/6883; G16B 25/00; C12Q 2600/112; C12Q 2600/156; C12Q 2600/158

(57) ABSTRACT

Described herein are systems and methods for identifying gene clusters in patients that have Crohn's Disease (CD). Further provided herein are systems and methods for determining or characterizing a Crohn's Disease (CD) subtype status in a subject having CD, selecting a treatment for a subject, or treating a subject.

### CROSS-REFERENCE TO RELATED APPLICATIONS

This application claims benefit of U.S. Provisional Patent Application No. 63/500,211, filed on May 4, 2023, and U.S. Provisional Patent Application No. 63/428,362, filed on Nov. 28, 2022, each of which is incorporated herein by reference in its entirety.

### STATEMENT AS TO FEDERALLY SPONSORED RESEARCH

This invention was made with government support under Grant Nos. DK043211, RR033176-01, and DK062413-18 awarded by the National Institutes for Health. The government has certain rights in the invention.

### BACKGROUND

In Crohn's disease (CD) extravasation of pro-inflammatory peripheral monocytes plays a fundamental role in innate immunity, gut homeostasis and modulating intestinal disease. Yet the molecular mechanisms contributing to chronic inflammation and disease progression mediated by circulating monocytes remains largely unknown.

### SUMMARY

In an aspect, a method of treating a Crohn's Disease (CD) subtype status is provided, wherein the status comprises a perianal fistula related CD14+ monocyte subtype (PAF-CD-mono subtype), the method comprising detecting expression of any one or more of the genes TNF, CCL4, CXCL3, CCL3, SELP, SELPLG, PROS1, PROC, MTR, CD226, CD155, and CXCL8 to obtain an expression profile; determining the CD subtype status based on the expression profile, wherein differential expression in the any one or more genes as compared to a reference expression profile indicates status of PAF-CD-mono subtype; and administering a therapeutically effective amount of a therapeutic agent for treating CD. In an aspect, a method of treating a Crohn's Disease (CD) subtype status is provided, wherein the status comprises a perianal fistula related CD14+ monocyte subtype (PAF-CD-mono subtype), the method comprising: detecting expression of any one or more the genes from Tables 1, 2, and 3 to obtain an expression profile; determining the CD subtype status based on the expression profile, wherein differential expression in the any one or more genes as compared to a reference expression profile indicates status of PAF-CD-mono subtype; and administering a therapeutically effective amount of a therapeutic agent for treating CD. In an aspect, a method of treating a Crohn's Disease (CD) subtype status is provided, wherein the status comprises a perianal fistula related CD14+ monocyte subtype (PAF-CD-mono subtype), the method comprising: detecting expression of any one or more the genes of the cell type cluster PAF-CD-mono to obtain an expression profile; determining the CD subtype status based on the expression profile, wherein differential expression in the any one or more genes as compared to a reference expression profile indicates status of PAF-CD-mono subtype; and administering a therapeutically effective amount of a therapeutic agent for treating CD. In an aspect, a method of treating a Crohn's Disease (CD) subtype status is provided, wherein the status comprises a perianal fistula related CD14+ monocyte subtype (PAF-CD-mono subtype), the method comprising: detecting expression of any one or more of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF to obtain an expression profile; determining the CD subtype status based on the expression profile, wherein differential expression of the any one or more genes as compared to a reference expression profile indicates status of PAF-CD-mono subtype; and administering a therapeutically effective amount of a therapeutic agent for treating CD. In embodiments, the one or more genes comprise CARD9, NOD2, TNFRSF1B, CD93, SELL, ITGAL, ITGAM, and IGTB2. In embodiments, the one or more genes comprise TLR1, TLR5, TRLR6, TRAF3, TLR8, TLR4, MYD88, and IRAK3. In embodiments, the one or more genes comprise VWF. In embodiments, differential expression in the any one or more genes comprises reduced expression in any one or more of the genes. In embodiments, differential expression in the any one or more genes comprises increased expression in any one or more of the genes. In embodiments, the therapeutic agent comprises an anti-TL1A antibody. In embodiments, the therapeutic agent comprises a steroid. In embodiments, the therapeutic agent comprises an immunosuppressant. In embodiments, the any one or more genes comprise any one or more of SELP and CD226. In embodiments, the any one or more of the genes from Tables 1, 2, and 3, comprise genes associated with any one or more of (i) platelet-monocyte interaction and clotting pathways, (ii) monocyte mediated inflammatory cytokine/chemokine expression, (iii) candidate TED-risk gene expression, and (iv) MDSC surface marker expression. In embodiments, the genes associated with candidate TED-risk gene expression comprise any one or more of PROS1, MTR, and PROC APC. In embodiments, the any one or more the genes from Tables 1, 2, and 3, comprises genes associated with any one or more of perianal fistula and perianal disease. In embodiments, the genes associated with any one or more of perianal fistula and perianal disease comprise any one or more of CD226, SELP, and PROS1. In embodiments, differential gene expression of the any one or more of the genes is due to aggregation of CD14+ monocytes with (i) platelets, (ii) megakaryocytes, or (iii) both platelets and megakaryocytes. In an aspect, a method of treating a Crohn's Disease (CD) subtype status in a subject is provided, wherein the status comprises a perianal fistula related CD14+ monocyte subtype (PAF-CD-mono subtype), the method comprising administering a therapeutically effective amount of a therapeutic agent for treating CD, provided that one or more polymorphisms comprising rs763361 or a proxy polymorphism in linkage disequilibrium therewith as determined with an r2 of at least 0.85, or a combination thereof, are detected in a biological sample obtained from the subject. In embodiments, (i) the one or more polymorphisms is detected using one or more of a microarray, sequencing, and qPCR; and/or (ii) the biological sample comprises a blood sample or is purified from a blood sample of the subject. In embodiments, the subject's genotype comprises a rs763361 T variant, optionally wherein the subject is homozygous for the rs763361 T variant.

In an aspect, a method of treating moderate to severe Crohn's Disease (CD) in a subject is provided, the method comprising: administering a therapeutically effective amount of a therapeutic agent for treatment of the CD, provided the subject is determined to have a perianal fistula related CD14+ monocyte subtype (PAF-CD-mono subtype) based, at least in part, on differential expression of one or more genes from Tables 1, 2, and 3 in a biological sample obtained from the subject, relative to a reference expression profile. In embodiments, the one or more genes from Tables 1, 2, or 3 comprise genes of the cell type cluster PAF-CD-mono. In embodiments, the one or more genes from Tables 1, 2, or 3 comprises any one or more of TNF, CCL4, CXCL3, CCL3, SELP, SELPLG, PROS1, PROC, MTR, CD226, CD155, and CXCL8. In embodiments, the one or more genes from Tables 1, 2, or 3 comprise any one or more of SELP and CD226. In an aspect, a method of treating moderate to severe Crohn's Disease (CD) in a subject is provided, the method comprising: administering a therapeutically effective amount of a therapeutic agent for treatment of the CD, provided the subject is determined to have a perianal fistula related CD14+ monocyte subtype (PAF-CD-mono subtype) based, at least in part, on differential expression (e.g., an increased expression) of one or more genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF in a biological sample obtained from the subject, relative to a reference expression profile. In embodiments, the one or more genes comprise CARD9, NOD2, TNFRSF1B, CD93, SELL, ITGAL, ITGAM, and IGTB2. In embodiments, the one or more genes comprise TLR1, TLR5, TRLR6, TRAF3, TLR8, TLR4, MYD88, and IRAK3. In embodiments, the one or more genes comprise VWF. In an aspect, a method of treating moderate to severe Crohn's Disease (CD) in a subject is provided, the method comprising: administering a therapeutically effective amount of a therapeutic agent for treatment of the CD, provided that one or more polymorphisms comprising rs763361 or a proxy polymorphism in linkage disequilibrium therewith as determined with an r² of at least 0.85, or a combination thereof, are detected in a biological sample obtained from the subject. In embodiments, the PAF-CD-mono subtype is associated with perianal disease or perianal fistula. In embodiments, the therapeutic agent comprises an anti-TL1A antibody. In embodiments, the therapeutic agent comprises a steroid. In embodiments, the therapeutic agent comprises an immunosuppressant.

In an aspect, a method of determining a Crohn's Disease (CD) subtype status is provided, wherein the status comprises a perianal fistula related CD14+ monocyte subtype (PAF-CD-mono subtype), the method comprising: detecting expression of any one or more of the genes TNF, CCL4, CXCL3, CCL3, SELP, SELPLG, PROS1, PROC, MTR, CD226, CD155, and CXCL8 to obtain an expression profile; and determining the CD subtype status based on the expression profile, wherein differential expression in the any one or more genes as compared to a reference expression profile indicates status of PAF-CD-mono subtype. In an aspect, a method of determining a Crohn's Disease (CD) subtype status is provided, wherein the status comprises a perianal fistula related CD14+ monocyte subtype (PAF-CD-mono subtype), the method comprising: detecting expression of any one or more of the genes from Tables 1, 2, and 3 to obtain an expression profile; and determining the CD subtype status based on the expression profile, wherein differential expression in the any one or more genes as compared to a reference expression profile indicates status of PAF-CD-mono subtype. In an aspect, a method of determining a Crohn's Disease (CD) subtype status, wherein the status comprises a perianal fistula related CD14+ monocyte subtype (PAF-CD-mono subtype), the method comprising: detecting expression of any one or more the genes of the cell cluster type PAF-CD-mono to obtain an expression profile; and determining the CD subtype status based on the expression profile, wherein differential expression in the any one or more genes as compared to a reference expression profile indicates status of PAF-CD-mono subtype. In an aspect, a method of determining a Crohn's Disease (CD) subtype status is provided, wherein the status comprises a perianal fistula related CD14+ monocyte subtype (PAF-CD-mono subtype), the method comprising: detecting expression of any one or more of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF to obtain an expression profile; and determining the CD subtype status based on the expression profile, wherein differential expression in the any one or more genes as compared to a reference expression profile indicates status of PAF-CD-mono subtype. In embodiments, the one or more genes comprise CARD9, NOD2, TNFRSF1B, CD93, SELL, ITGAL, ITGAM, and IGTB2. In embodiments, the one or more genes comprise TLR1, TLR5, TRLR6, TRAF3, TLR8, TLR4, MYD88, and IRAK3. In embodiments, the one or more genes comprise VWF. In embodiments, differential expression in the any one or more of the genes comprises reduced expression in any one or more of the genes. In embodiments, differential expression in the any one or more of the genes comprise increased expression in any one or more of the genes. In embodiments, any one or more of the genes comprise any one or more of SELP and CD226. In embodiments, any one or more of the genes from Tables 1, 2, and 3, comprise genes associated with any one or more of (i) platelet-monocyte interaction and clotting pathways, (ii) monocyte mediated inflammatory cytokine/chemokine expression, (iii) candidate TED-risk gene expression, and (iv) MDSC surface marker expression. In embodiments, the genes associated with candidate TED-risk gene expression, comprise any one or more of PROS1, MTR, PROC APC. In embodiments, the any one or more of the genes from Tables 1, 2, and 3, comprises genes associated with any one or more of perianal fistula and perianal disease. In embodiments, the genes associated with any one or more of perianal fistula and perianal disease comprise any one or more of CD226, SELP, and PROS1. In embodiments, differential gene expression of any one or more of the genes is due to aggregation of CD14+ monocytes with (i) platelets, (ii) megakaryocytes, or (iii) both platelets and megakaryocytes. In an aspect, a method of determining a Crohn's Disease (CD) subtype status is provided, wherein the status comprises a perianal fistula related CD14+ monocyte subtype (PAF-CD-mono subtype), the method comprising: detecting the presence of one or more polymorphisms comprising rs763361 or a proxy polymorphism in linkage disequilibrium therewith as determined with an r² of at least 0.85, or a combination thereof, in a biological sample obtained from the subject; and determining the CD subtype status based on the presence of the one or more polymorphisms, wherein the presence of the one or more polymorphisms indicates status of PAF-CD-mono subtype.

In embodiments, the reference expression profile of any of the reference expression profiles described herein is derived from gene expression levels measured in samples obtained from one or more individuals that: (i) does not have CD; or (ii) has a subtype of CD that is not characterized by PAF-CD-mono.

In an aspect, a method for processing or analyzing a biological sample from a subject is provided, comprising: (i) obtaining the biological sample comprising gene expression products, wherein the subject has or is suspected of having Crohn's Disease (CD); (ii) subjecting the biological sample to an assay by sequencing, array hybridization, and/or nucleic acid amplification to yield a data set including data corresponding to gene expression product levels; (iii) in a programmed computer, inputting said data including said gene expression product levels from (ii) to a trained algorithm to generate a classification of said sample as positive or negative for a PAF-CD-mono subtype, wherein the trained algorithm is trained with a plurality of training samples, and wherein said biological sample is independent of said plurality of training samples; and (iv) electronically outputting a report that identifies the classification of the biological sample as positive or negative for the PAF-CD-mono subtype. In embodiments, the gene expression products comprise any one or more of TNF, CCL4, CXCL3, CCL3, SELP, SELPLG, PROS1, PROC, MTR, CD226, CD155, and CXCL8. In embodiments, the gene expression products comprise (i) SELP, (ii) CD226 or (iii) both SELP and CD226. In embodiments, the gene expression products comprise any one or more genes from Tables 1, 2, and 3. In embodiments, the gene expression products comprise any one or more of CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF. In embodiments, the gene expression products comprise any one or more of CARD9, NOD2, TNFRSF1B, CD93, SELL, ITGAL, ITGAM, and IGTB2. In embodiments, the gene expression products comprise any one or more of TLR1, TLR5, TRLR6, TRAF3, TLR8, TLR4, MYD88, and IRAK3. In embodiments, the gene expression products comprise any one or more of comprise VWF. In embodiments, the gene expression products comprise any one or more genes of cell type cluster PAF-CD-mono.

In an aspect, a system comprising a kit for determining a Crohn's Disease (CD) subtype status in a subject having CD is provided, the system comprising: one or more detection reagents comprising nucleic acids configured to hybridize to any one or more genes TNF, CCL4, CXCL3, CCL3, SELP, SELPLG, PROS1, PROC, MTR, CD226, CD155, and CXCL8. In an aspect, a system comprising a kit for determining a Crohn's Disease (CD) subtype status in a subject having CD is provided, the system comprising: one or more detection reagents comprising nucleic acids configured to hybridize to any one or more genes in Tables 1, 2, and 3. In an aspect, a system comprising a kit for determining a Crohn's Disease (CD) subtype status in a subject having CD is provided, the system comprising: one or more detection reagents comprising nucleic acids configured to hybridize to CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF. In an aspect, a system comprising a kit for determining a Crohn's Disease (CD) subtype status in a subject having CD is provided, the system comprising: one or more detection reagents comprising nucleic acids configured to hybridize to one or more polymorphisms comprising rs763361 or a proxy polymorphism in linkage disequilibrium therewith as determined with an r2 of at least 0.85, or a combination thereof. In an aspect, a system comprising a kit for determining a Crohn's Disease (CD) subtype status in a subject having CD is provided, the system comprising: one or more detection reagents comprising nucleic acids configured to hybridize to any one or more genes of cell type cluster PAF-CD-mono. In embodiments, the kit comprises reagents for use in a qPCR reaction. In embodiments, the one or more detection reagents comprise one or more primers or probe. In embodiments, the probe comprises a quencher. In embodiments, the probe comprises a detectable label. In embodiments, the kit further comprises a chip comprising a solid substrate functionalized with oligonucleotides that hybridize to at least a portion of a sequence of the genes TNF, CCL4, CXCL3, CCL3, SELP, SELPLG, PROS1, PROC, MTR, CD226, CD155, and CXCL8. In embodiments, the oligonucleotides that hybridize to at least a portion of a sequence of the genes (i) SELP, (ii) CD226, or (iii) both SELP and CD226. In embodiments, the kit further comprises a chip comprising a solid substrate functionalized with oligonucleotides that hybridize to at least a portion of a sequence of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF. In embodiments, the genes comprise CARD9, NOD2, TNFRSF1B, CD93, SELL, ITGAL, ITGAM, and IGTB2. In embodiments, the genes comprise TLR1, TLR5, TRLR6, TRAF3, TLR8, TLR4, MYD88, and IRAK3. In embodiments, the genes comprise VWF. In embodiments, the kit further comprises a chip comprising a solid substrate functionalized with oligonucleotides that hybridize to at least a portion of a sequence of the any one or more genes in Tables 1, 2, and 3. In embodiments, the kit further comprises a chip comprising a solid substrate functionalized with oligonucleotides that hybridize to at least a portion of a sequence of the any one or more genes of cell type cluster PAF-CD-mono.

In an aspect, a computer-implemented platform for determining a Crohn's Disease (CD) subtype status in a subject having CD is provided, wherein the status comprises identifying a perianal fistula related CD14+ monocyte subtype (PAF-CD-mono subtype), the computer-implemented platform comprising: one or more processors collectively or individually programmed to implement a method comprising: (i) analyzing gene expression data of the subject to detect a level of expression of any one or more genes TNF, CCL4, CXCL3, CCL3, SELP, SELPLG, PROS1, PROC, MTR, CD226, CD155, and CXCL8, to produce an expression profile of the subject; and (ii) determining the CD subtype status of the subject based upon the expression profile, wherein differential expression of the one or more genes as compared to a reference expression profile indicates that the CD subtype status of the subject comprises a PAF-CD-mono subtype; and a database for storing the gene expression data of the subject and/or the expression profile. In embodiments, analyzing the gene expression data of the subject to detect the level of expression of any one or more of the genes comprises detecting the level of expression of (i) SELP, (ii) CD226, or (iii) both SELP and CD226. In an aspect, a computer-implemented platform for determining a Crohn's Disease (CD) subtype status in a subject having CD is provided, wherein the status comprises identifying a perianal fistula related CD14+ monocyte subtype (PAF-CD-mono subtype), the computer-implemented platform comprising: one or more processors collectively or individually programmed to implement a method comprising: (i) analyzing gene expression data of the subject to detect a level of expression of any one or more genes in Tables 1, 2, and 3, to produce an expression profile of the subject; and (ii) determining the CD subtype status of the subject based upon the expression profile, wherein differential expression of the one or more genes as compared to a reference expression profile indicates that the CD subtype status of the subject comprises a PAF-CD-mono subtype; and a database for storing the gene expression data of the subject and/or the expression profile. In embodiments, any one or more the genes in Tables 1, 2, and 3 comprise (i) SELP, (ii) CD226, or (iii) both SELP and CD226. In an aspect, a computer-implemented platform for determining a Crohn's Disease (CD) subtype status in a subject having CD is provided, wherein the status comprises identifying a perianal fistula related CD14+ monocyte subtype (PAF-CD-mono subtype), the computer-implemented platform comprising: one or more processors collectively or individually programmed to implement a method comprising: (i) analyzing gene expression data of the subject to detect a level of expression of any one or more genes of cell type cluster PAF-CD-mono, to produce an expression profile of the subject; and (ii) determining the CD subtype status of the subject based upon the expression profile, wherein differential expression of the one or more genes as compared to a reference expression profile indicates that the CD subtype status of the subject comprises a PAF-CD-mono subtype; and a database for storing the gene expression data of the subject and/or the expression profile. In embodiments, any one or more the genes of the cell type cluster PAF-CD-mono comprise (i) SELP, (ii) CD226, or (iii) both SELP and CD226. In an aspect, a computer-implemented platform for determining a Crohn's Disease (CD) subtype status in a subject having CD is provided, wherein the status comprises identifying a perianal fistula related CD14+ monocyte subtype (PAF-CD-mono subtype), the computer-implemented platform comprising: one or more processors collectively or individually programmed to implement a method comprising: (a) analyzing gene expression data of the subject to detect a level of expression of any one or more genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF, to produce an expression profile of the subject; and (b) determining the CD subtype status of the subject based upon the expression profile, wherein differential expression of the one or more genes as compared to a reference expression profile indicates that the CD subtype status of the subject comprises a PAF-CD-mono subtype; and a database for storing the gene expression data of the subject and/or the expression profile. In embodiments, the one or more genes comprise CARD9, NOD2, TNFRSF1B, CD93, SELL, ITGAL, ITGAM, and IGTB2. In embodiments, the one or more genes comprise TLR1, TLR5, TRLR6, TRAF3, TLR8, TLR4, MYD88, and IRAK3. In embodiments, the one or more genes comprise VWF. In embodiments, the computer implemented platform further comprises a device for detecting the expression of any one or more genes in a biological sample obtained from the subject. In embodiments, the device comprises a microarray, a sequencer, or a qPCR machine. In embodiments, the expression profile is predictive of the PAF-CD-mono subtype with an accuracy of at least 70%, 80%, 90%, or 100%. In embodiments, the expression profile is predictive of the PAF-CD-mono subtype with an area under the curve (AUC) of at least about 0.70, 0.80, 0.90, or 1.0. In embodiments, the expression profile is predictive of PAF-CD-mono subtype with a negative predictive value (NPL) of at least 70%, 80%, 90%, or 100%. In embodiments, the expression profile is predictive of PAF-CD-mono subtype with a positive predictive value (PPV) of at least 70%, 80%, 90%, or 100%. In embodiments, the CD-MNP subtype is characterized by a PAF-CD-mono transcriptomic signature. In embodiments, the PAF-CD-mono transcriptomic subtype is associated with perianal disease or perianal fistula. In embodiments, the reference expression profile is derived from gene expression levels measured in samples obtained from one or more individuals that: (i) does not have CD or (ii) has a subtype of CD that is not characterized by PAF-CD-mono. In embodiments, differential expression of the one or more genes comprises reduced expression in any one or more the genes. In embodiments, differential expression of the one or more genes comprises increased expression in any one or more of the genes.

In an aspect, a method of treating a Crohn's Disease (CD) subtype status is provided, wherein the status comprises a perianal fistula related CD14+ monocyte subtype (PAF-CD-mono subtype), the method comprising: detecting expression of (i) SELP, (ii) CD226, or (iii) both SELP and CD226; determining the CD subtype status based on detecting expression of SELP, CD226, or both SELP and CD226, wherein differential expression of SELP, CD226, or both SELP and CD226, as compared to a reference expression profile indicates status of PAF-CD-mono subtype; and administering a therapeutically effective amount of a therapeutic agent for treating CD. In embodiments, differential expression of SELP, CD226, or both SELP and CD226 comprises reduced expression of SELP, CD226, or both SELP and CD226. In embodiments, the therapeutic agent comprises an anti-TL1A antibody. In embodiments, the therapeutic agent comprises a steroid. In embodiments, the therapeutic agent comprises an immunosuppressant. In embodiments, differential expression of SELP, CD226, or both SELP and CD226 is due to aggregation of CD14+ monocytes with (i) platelets, (ii) megakaryocytes, or (iii) both platelets and megakaryocytes. In embodiments, SELP, CD226, or both SELP and CD226 are associated with any one or more of perianal fistula and perianal disease. In embodiments, the reference expression profile is derived from gene expression levels measured in samples obtained from one or more individuals that: (i) does not have CD or (ii) has a subtype of CD that is not characterized by PAF-CD-mono.

In an aspect, a method of treating moderate to severe Crohn's Disease (CD) in a subject is provided, the method comprising: administering a therapeutically effective amount of a therapeutic agent for treatment of the CD, provided the subject is determined to have a perianal fistula related CD14+ monocyte subtype (PAF-CD-mono subtype) based, at least in part, on differential expression of (i) SELP, (ii) CD226, or (iii) both SELP and CD226 in a biological sample obtained from the subject, relative to a reference expression profile. In embodiments, differential expression of SELP, CD226, or both SELP and CD226 comprises reduced expression of SELP, CD226, or both SELP and CD226. In embodiments, the PAF-CD-mono subtype is associated with any one or more of perianal disease or perianal fistula. In embodiments, the therapeutic agent comprises an anti-TL1A antibody. In embodiments, the therapeutic agent comprises a steroid. In embodiments, the therapeutic agent comprises an immunosuppressant. In embodiments, differential expression of SELP, CD226, or both SELP and CD226 is due to aggregation of CD14+ monocytes with (i) platelets, (ii) megakaryocytes, or (iii) both platelets and megakaryocytes. In embodiments, differential expression of SELP, CD226, or both SELP and CD226 is associated with any one or more of perianal fistula and perianal disease. In embodiments, the reference expression profile is derived from gene expression levels measured in samples obtained from one or more individuals that: (i) does not have CD or (ii) has a subtype of CD that is not characterized by PAF-CD-mono.

In an aspect, a method of determining a Crohn's Disease (CD) subtype status is provided, wherein the status comprises a perianal fistula related CD14+ monocyte subtype (PAF-CD-mono subtype), the method comprising: detecting expression of (i) SELP, (ii) CD226, or (iii) both SELP and CD226; and determining the CD subtype status based on the expression of (i), (ii), or (iii), wherein differential expression in the any one or more of SELP, CD226, or both SELP and CD226 as compared to a reference expression profile indicates status of PAF-CD-mono subtype. In embodiments, differential expression of SELP, CD226, or both SELP and CD226 comprises reduced expression of SELP, CD226, or both SELP and CD226. In embodiments, differential expression of SELP, CD226, or both SELP and CD226 is due to aggregation of CD14+ monocytes with (i) platelets, (ii) megakaryocytes, or (iii) both platelets and megakaryocytes. In embodiments, the PAF-CD-mono subtype is associated with any one or more of perianal disease or perianal fistula. In embodiments, the reference expression profile is derived from gene expression levels measured in samples obtained from one or more individuals that: (i) does not have CD or (ii) has a subtype of CD that is not characterized by PAF-CD-mono.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1A provides results from a principal component analysis (PCA) of RNA-seq data for CD14+ monocytes isolated from CD patients at the time of surgery.

FIG. 1B provides results from a clustering analysis of total RNA-seq, which illustrates distinct CD14+ clusters.

FIG. 1C provides a heat map of genes differentially regulated greater than or equal to 1.5-fold in the CD14+ cluster 1 and CD14+ cluster 2 from FIG. 1B.

FIG. 1D provides a heat map of genes differentially regulated greater than or equal to 2.0-fold in the CD14+ cluster 1 and the CD14+ cluster 2 from FIG. 1B.

FIG. 2A provides ENRICHR pathway analysis of the differentially expressed genes.

FIG. 2B provides Reactome pathway analysis showing that CD-mono differential gene expression is associated with platelet activation and clotting pathways.

FIG. 2C provides GO biological process pathway analysis showing that CD-mono differential gene expression is associated with platelet activation, adhesion, and wound healing pathways.

FIG. 3 provides an ARCHS4 generated t-SNE plots showing that gene signature from a differentially upregulated gene panel in PAF-CD-mono vs CD-mono overlaps with similar co-expression from thrombocytes.

FIG. 4 provides results showing differential gene expression in CD14+ cluster subtypes with monocyte-platelet associated pro-inflammatory/cytotoxic gene expression.

FIG. 5 provides results showing gene expression in CD14+ cluster subtypes of genes associated with monocyte-platelet associated complex and clotting associated genes.

FIG. 6 provides results showing differential gene expression in CD14+ cluster subtypes with CD226 (DNAM-1) and its receptor, PVR.

FIG. 7 provides results showing that there is no association of CBC lab values or other clinical, demographic markers associated with the CD14+ cluster subtype.

FIG. 8 provides results showing that a CD14+ subset is associated with Perianal Fistula and a higher disease-severity score mediated largely by the fistula component, with association lost following exclusion of fistula from scoring.

FIG. 9 provides results showing that decreased gene expression in the PAF-CD-mono subtype was associated with perianal fistula.

FIG. 10 provides results showing that CD226 gene expression is correlated with thromboembolic associated (TED) associated genes.

FIG. 11 provides results showing that low expression of CD226 and SELP is associated with perianal fistula and B2/B3 disease behavior.

FIG. 12A provides a heatmap of genes differentially expressed in monocytes between CD patients with (n=10) or without (n=42) perianal fistula.

FIG. 12B provides a pathway analysis of the differentially expressed genes.

FIG. 13A provides hierarchical clustering demonstrating a distinct gene expression pattern of the CD14+CD-mono subtype versus a CD14+ PAF-CD-mono subtype in confirmational cohorts, which includes a first cohort of 15 repeat patient samples from FIGS. 1A-1B and FIG. 2A and a second cohort of 16 new patient samples. FIG. 13A demonstrates a distinct gene expression pattern of the CD14+CD-mono subtype versus a CD14+ PAF-CD-mono subtype in confirmational cohorts, which includes a first cohort of 15 repeat patient samples from FIGS. 1A-1B and FIG. 2A and a second cohort of 16 new patient samples. Among the first cohort of 15 repeat patient samples, 14 of the 15 samples classified as they did in FIGS. 1A-1B and FIG. 2A. When the 15 repeat samples of the first cohort were removed, 15 of the 16 new samples in the second cohort clustered the same way as shown in FIG. 13A.

FIG. 13B provides a heatmap demonstrating a distinct gene expression pattern of the CD14+CD-mono subtype versus a CD14+ PAF-CD-mono subtype from hierarchical clusters shown in FIG. 13A.

FIG. 14A provides results showing that perianal fistula is associated with the PAF-CD-mono subtype in a validation cohort (a cohort of patients not included in FIG. 8 and FIG. 11).

FIG. 14B provides results showing that reduced expression of certain monocyte-platelet markers are associated with perianal fistula in the same validation cohort.

FIG. 15A provides results showing that perianal disease is associated with the PAF-CD-mono subtype, after two (2) independent RNA-seq runs combining the initial cohort tested in FIG. 8 and FIG. 11 and the validation cohort tested in FIGS. 14A-14B.

FIG. 15B provides results showing that perianal fistula is associated with the PAF-CD-mono subtype, after two (2) independent RNA-seq runs of the same combined cohort.

FIG. 16A provides results showing differential gene expression of platelet-mediated monocyte aggregation, activation, and signaling genes in PAF-CD-mono patients.

FIG. 16B provides additional results showing differential gene expression of platelet-mediated monocyte aggregation, activation, and signaling genes in PAF-CD-mono patients.

FIG. 17 provides results showing that perianal fistula is associated with platelet-mediated monocyte aggregation, activation, signaling, and CFB interactive marker genes.

FIG. 18 provides results showing that low expression of CD226 and SELP is associated with perianal fistula.

FIG. 19 provides a schematic of an analysis used for identifying CD14+ subtypes.

FIG. 20 provide results showing that certain monocyte-platelet markers have altered gene expression following surgery.

FIG. 21 shows that there is a decrease in CD226 and SELP post-surgery in the CD-mono while an increase is detected for the PAF subjects.

FIG. 22A shows the association of perianal fistula with expression of the monocyte-platelet marker CD226, before and after surgery.

FIG. 22B shows the association of perianal fistula with expression of the monocyte-platelet marker SELP, before and after surgery.

FIG. 23 provides results showing that circulating CD226 protein mirrors CD226 mRNA expression in both the CD-mono and PAF-CD-mono subtypes.

FIG. 24 provides results showing that low levels of CD226 protein levels are associated with perianal fistula, structuring disease, and family history.

FIG. 25 provides a heatmap showing differential gene expression between the CD14+ clusters for any gene that had at least a 2-fold change in gene expression relative to the control group as further described in Table 1.

FIG. 26 provides a heatmap showing differential gene expression between the CD14+ clusters for any gene that had at least a 1.5-fold change in gene expression relative to the control group as further described in Table 2.

FIG. 27 provides a heat map showing differential gene expression of genes that are within the IBD GWAS loci, which had at least a 1.5-fold change in gene expression relative to the control group as further described in Table 3.

FIG. 28 provides results showing that CD226 rs763361 was associated with decreased expression of CD226 (left panel) and perianal fistula (right panel).

FIG. 29 provides Reactome pathway analysis showing that CD-PAF upregulated differential gene expression was associated with innate immune regulation.

FIG. 30 provides results showing that innate immune response and adhesion markers including CARD9, NOD2, TNFRSF1B, CD93, SELL, ITGAL, ITGAM, and IGTB2 were elevated in PAF-CD-mono patients as compared to CD-mono patients.

FIG. 31 provides results showing that expression of Toll-like receptor (TLR) signaling molecules including TLR1, TLR5, TRLR6, TRAF3, TLR8, TLR4, MYD88, and IRAK3 were elevated in PAF-CD-mono patients as compared to CD-mono patients.

### DETAILED DESCRIPTION

The present disclosure provides methods and systems for characterizing and treating Crohn's Disease (CD). Intestinal monocytes play a key role in innate immunity, gut homeostasis and intestinal disease, and monocyte activation contributes to chronic inflammation and disease progression in CD. Therefore, there is a need to characterize the monocyte signatures to provide more effective therapeutic strategies based on transcriptomic profiles. In particular embodiments, a CD patient is characterized as having or not having perianal fistula related signature in CD14+ monocytes by transcriptomic profiling (“PAF-CD-mono signature”). Such PAF-CD-mono signature can include a signature of genes associated with platelet activation, a signature of genes associated with clotting pathways, and/or a signature of genes associated with thrombocytes. A patient having either one of these signatures may express one or more genes disclosed herein, e.g., one or more genes of Tables 1, 2, and 3 at a level higher or lower than a reference subject as indicated in Tables 1, 2, and 3. The reference subject may be a subject that does not have IBD. The reference subject may be a subject that does not have CD or a severe form of CD. The reference subject may be a subject that does not have a PAF-CD-mono subtype of CD. In other embodiments, one or more polymorphisms comprising rs763361 or a proxy polymorphism in linkage disequilibrium therewith as determined with an r² of at least 0.85, or a combination thereof, can be used to determine whether a CD patient has a PAF-CD-mono subtype.

In an aspect, a method is provided comprising treating a Crohn's Disease (CD) subtype status, wherein the status comprises a perianal fistula related CD14+ monocyte subtype (PAF-CD-mono subtype), the method comprising detecting expression of any one or more genes to obtain an expression profile; determining the CD subtype status based on the expression profile, wherein differential expression in the any one or more genes as compared to a reference expression profile indicates status of PAF-CD-mono subtype; and administering a therapeutically effective amount of a therapeutic agent for treating CD.

In an aspect, a method is provided comprising treating a Crohn's Disease (CD) subtype status, wherein the status comprises a perianal fistula related CD14+ monocyte subtype (PAF-CD-mono subtype), the method comprising administering a therapeutically effective amount of a therapeutic agent for treating CD, provided that one or more polymorphisms comprising rs763361 or a proxy polymorphism in linkage disequilibrium therewith as determined with an r2 of at least 0.85, or a combination thereof, are detected in a biological sample obtained from the subject.

In an aspect, a method is provided comprising treating a Crohn's Disease (CD) subtype status, wherein the status comprises a perianal fistula related CD14+ monocyte subtype (PAF-CD-mono subtype), the method comprising detecting one or more polymorphisms comprising rs763361 or a proxy polymorphism in linkage disequilibrium therewith as determined with an r2 of at least 0.85, or a combination thereof, in a biological sample obtained from the subject, and administering a therapeutically effective amount of a therapeutic agent for treating CD.

In an aspect, a method of treating moderate to severe Crohn's Disease (CD) in a subject is provided, comprising administering a therapeutically effective amount of a therapeutic agent for treatment of the CD, provided the subject is determined to have a perianal fistula related CD14+ monocyte subtype (PAF-CD-mono subtype) based, at least in part, on differential expression of any one or more genes in a biological sample obtained from the subject, relative to a reference expression profile.

In an aspect, a method of treating moderate to severe Crohn's Disease (CD) in a subject is provided, comprising administering a therapeutically effective amount of a therapeutic agent for treatment of the CD, provided the subject is determined to have a perianal fistula related CD14+ monocyte subtype (PAF-CD-mono subtype) based, at least in part, on detection of one or more polymorphisms comprising rs763361 or a proxy polymorphism in linkage disequilibrium therewith as determined with an r2 of at least 0.85, or a combination thereof, in a biological sample obtained from the subject.

In an aspect, a method of determining a Crohn's Disease (CD) subtype status is provided, wherein the status comprises a perianal fistula related CD14+ monocyte subtype (PAF-CD-mono subtype), the method comprising: detecting expression of any one or more the genes of the cell cluster type PAF-CD-mono to obtain an expression profile; and determining the CD subtype status based on the expression profile, wherein differential expression in the any one or more genes as compared to a reference expression profile indicates status of PAF-CD-mono subtype.

In embodiments, the any one or more genes comprises any one or more of TNF, CCL4, CXCL3, CCL3, SELP, SELPLG, PROS1, PROC, MTR, CD226, CD155, and CXCL8. In embodiments, the any one or more genes comprises any two (2) or more, three (3) or more, four (4) or more, five (5) or more, six (6) or more, seven (7) or more, eight (8) or more, nine (9) or more, or ten (10) of TNF, CCL4, CXCL3, CCL3, SELP, SELPLG, PROS1, PROC, MTR, CD226, CD155, and CXCL8. In embodiments, the any one or more genes comprises each of TNF, CCL4, CXCL3, CCL3, SELP, SELPLG, PROS1, PROC, MTR, CD226, CD155, and CXCL8.

In embodiments, the any one or more genes comprises TNF. In embodiments, the any one or more genes comprises CCL4. In embodiments, the any one or more genes comprises CXCL3. In embodiments, the any one or more genes comprises CCL3. In embodiments, the any one or more genes comprises SELP. In embodiments, the any one or more genes comprises SELPLG. In embodiments, the any one or more genes comprises PROS1. In embodiments, the any one or more genes comprises PROC. In embodiments, the any one or more genes comprises MTR. In embodiments, the any one or more genes comprises CD226. In embodiments, the any one or more genes comprises CD155. In embodiments, the any one or more genes comprises CXCL8. In embodiments, the any one or more genes comprises SELP and CD226.

In embodiments, the any one or more genes comprises any one or more genes from Tables 1, 2, and 3.

In embodiments, the any one or more genes comprises any one or more of CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF. In embodiments, the any one or more genes comprises any two (2) or more, three (3) or more, four (4) or more, five (5) or more, six (6) or more, seven (7) or more, or eight (8) of CARD9, NOD2, TNFRSF1B, CD93, SELL, ITGAL, ITGAM, IGTB2. In embodiments, the any one or more genes comprises any two (2) or more, three (3) or more, four (4) or more, five (5) or more, six (6) or more, seven (7) or more, or eight (8) of TLR1, TLR5, TRLR6, TRAF3, TLR8, TLR4, MYD88, and IRAK3. In embodiments, the any one or more genes comprises each of TLR1, TLR5, TRLR6, TRAF3, TLR8, TLR4, MYD88, and IRAK3. In embodiments, the any one or more genes comprises each of CARD9, NOD2, TNFRSF1B, CD93, SELL, ITGAL, ITGAM, IGTB2, TLR1, TLR5, TRLR6, TRAF3, TLR8, TLR4, MYD88, and IRAK3. In embodiments, the any one or more genes comprises VWF. In embodiments, the any one or more genes comprise any one or more of CLU, ITGA2B, MGLL, PROS1, GP1BA, GP9, SPARC, and YES1.

In embodiments, the any one or more genes comprises any one or more genes from the cell type cluster PAF-CD-mono.

In some embodiments, SELP is decreased in PAF-CD-mono of a subject of PAF-CD-mono subtype subtype as compared to that in the CD14+ monocyte of a CD subject not affected by perianal fistula. In another embodiment, CD226 is decreased in PAF-CD-mono of a subject of PAF-CD-mono subtype subtype as compared to that in the CD14+ monocyte of a CD subject not affected by perianal fistula. In embodiments, both SELP and CD226 are decreased in PAF-CD-mono of a subject of PAF-CD-mono subtype as compared to that in the CD14+ monocyte of a CD subject not affected by perianal fistula. Additional markers that are decreased in PAF-CD-mono of a subject of PAF-CD-mono subtype subtype compared to that in the CD14+ monocyte of a CD subject not affected by perianal fistula, include any one or more of PROS1, MTR, CXCL3, CXCL8, TNF, CCL3, and CCL4.

In some embodiments, SELP is decreased in a subject of PAF-CD-mono subtype subtype as compared to that in a CD subject not affected by perianal fistula. In another embodiment, CD226 is decreased in a subject of PAF-CD-mono subtype subtype as compared to that in a CD subject not affected by perianal fistula. Additional markers that are decreased in a subject of PAF-CD-mono subtype subtype as compared to that in a CD subject not affected by perianal fistula, include any one or more of PROS1, MTR, CXCL3, CXCL8, TNF, CCL3, and CCL4.

In some embodiments, SELPLG is increased in PAF-CD-mono of a subject of PAF-CD-mono subtype subtype as compared to that in the CD14+ monocyte of a CD subject not affected by perianal fistula. Additional markers that are increased in PAF-CD-mono of a subject of PAF-CD-mono subtype subtype as compared to that in the CD14+ monocyte of a CD subject not affected by perianal fistula, include any one or more of PROC and CD155.

In some embodiments, SELPLG is increased in a subject of PAF-CD-mono subtype subtype as compared to that in a CD subject not affected by perianal fistula. Additional markers that are increased in a subject of PAF-CD-mono subtype subtype as compared to that in a CD subject not affected by perianal fistula, include any one or more of PROC and CD155.

In embodiments, differential expression in the any one or more genes comprises increased expression in the any one or more genes relative to a reference subject. In embodiments, increased expression comprises at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least-2.4 fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, or at least 5.0-fold higher gene expression than a reference subject. In embodiments, increased expression comprises greater than 5.0-fold higher gene expression than a reference subject.

In embodiments, differential expression in the any one or more genes comprises decreased expression in the any one or more genes relative to a reference subject. In embodiments, decreased expression comprises at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, or at least 5.0-fold lower gene expression than a reference subject. In embodiments, decreased expression comprises greater than 5.0-fold lower gene expression than a reference subject.

In embodiments, differential expression in any one or more genes comprises increased expression in at least one gene relative to a reference subject and decreased expression in at least one (1) gene relative to a reference subject. In embodiments, increased expression in the at least one (1) gene comprises at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, or at least 5.0-fold higher gene expression than a reference subject. In embodiments, increased expression in the at least one (1) gene comprises greater than 5.0-fold higher gene expression than a reference subject. In embodiments, decreased expression in the at least one (1) gene comprises at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, or at least 5.0-fold lower gene expression than a reference subject. In embodiments, decreased expression in the at least one (1) gene comprises greater than 5.0-fold lower gene expression than a reference subject.

In embodiments, differential expression of any one or more of the genes describe herein is due to an innate immune response in which genes are differentially expressed in CD14+ monocytes relative to a reference expression profile. In some embodiments, the genes differentially expressed in CD14+ monocytes due to the innate immune response comprise any one or more of genes in Tables 1, 2, and 3. In some embodiments, the genes differentially expressed in CD14+ monocytes due to the innate immune response comprise (i) SELP, (ii) CD226, or (iii) both SELP and CD226. In some embodiments, the differentially expressed genes in CD14+ monocytes to the innate immune response comprise reduced expression of SELP and CD226.

In embodiments, differential expression of any of the genes described herein is due to differentially expressed genes in CD14+ monocytes as a result of activation of CD14+ monocytes by platelets. In some embodiments, activation of CD14+ monocytes by platelets is due to aggregation of CD14+ monocytes with (i) platelets, (ii) megakaryocytes, or (iii) both platelets and megakaryocytes. In some embodiments, aggregation of CD14+ monocytes with (i), (ii), or (iii) causes a platelet-mediated thrombotic signal in which genes are differentially regulated in CD14+ monocytes that function in the clotting cascade. In some embodiments, activation of CD14+ monocytes by platelets results in differential gene expression of any one or more the genes in Tables 1, 2, and 3. In some embodiments, activation of CD14+ monocytes by platelets results in differential gene expression of (i) SELP, (ii) CD226, or (iii) both SELP and CD226. In some embodiments, activation of CD14+ monocytes by platelets results in reduced expression of SELP and CD226.

In embodiments, differential expression of any one or more the genes described herein is due to differential gene expression in platelets. In some embodiments, differential gene expression in platelets is concomitant with aggregation of CD14+ monocytes with platelets. In some embodiments, differential gene expression in platelets comprises differential gene expression of SELP. In some embodiments, differential gene expression of SELP in platelets comprises reduced expression of SELP in platelets.

In embodiments, any of therapeutic agents for treating CD described herein comprises an anti-TL1A antibody. In some embodiments, the therapeutic comprise any one of the anti-TL1A antibodies described in U.S. Pat. Nos. 10,322,174; 10,689,439; 11,440,954; 11,292,848; 9,683,998; 10,968,279; 8,642,741; 10,822,422; 8,263,743; 10,138,296; 9,290,576; and 9,068,003; and US Patent Publication No. US 2022-0213226 (Ser. No. 17/379,968). In one embodiment, the anti-TL1A antibody comprises or consists of PRA023. In one embodiment, the anti-TL1A antibody comprises or consists of tulisokibart. In one embodiment, the anti-TL1A antibody comprises or consists of PF-06480605 (e.g., as disclosed in clinicaltrials.gov IDs NCT04090411, NCT05471492, and NCT02840721). In one embodiment, the anti-TL1A antibody comprises or consists of TEV-48574 (e.g., as disclosed in clinicaltrials.gov ID NCT05499130). All references cited in this paragraph are herein incorporated in their entireties by reference.

Alternatively, in some embodiments, the therapeutic agent for treating CD comprises an anti-CD30L antibody. In some embodiments, the therapeutic comprise any one of the anti-CD30L antibodies described in U.S. Pat. No. 9,926,373 and PCT Publication No. WO 2022/177963 (PCT Patent Application No. PCT/US2022/016565). In one embodiment, the anti-CD30L antibody comprises or consists of KPL-045. In another embodiment, the anti-CD30L antibody comprises or consists of PRA052. All references cited in this paragraph are herein incorporated in their entireties by reference.

In embodiments, the therapeutic agent for treating CD comprises adalimumab. In embodiments, the therapeutic agent for treating CD comprises infliximab. In embodiments, the therapeutic agent for treating CD comprises vedolizumab. In embodiments, the therapeutic agent for treating CD comprises ustekinumab.

In embodiments, the therapeutic agent for treating CD comprises a steroid.

In embodiments, the therapeutic agent for treating CD comprises an immunosuppressant. In embodiments, the immunosuppressant comprises azathioprine. In embodiments, the immunosuppressant comprises mercaptopurine. In embodiments, the immunosuppressant comprises methotrexate.

### Transcriptomic Signature and Profiling

In some embodiments, the methods involve detecting in a biological sample from a subject expression levels of one or more genes of a transcriptomic signature to obtain an expression profile comprising the expression levels of each of the one or more genes in the signature. In some embodiments, the transcriptomic signature comprises one or more biomarkers listed in Tables 1, 2, or 3. In some embodiments, the transcriptomic signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80, 90, 100, or more of the genes of Tables 1, 2, or 3. In some embodiments, the transcriptomic signature comprises any combination of 1, 2, 3, 4, 5, 6, 7, or 8 genes selected from CARD9, NOD2, TNFRSF1B, CD93, SELL, ITGAL, ITGAM, and IGTB2. In some embodiments, the transcriptomic signature comprises any combination of 1, 2, 3, 4, 5, 6, 7, or 8 genes selected from TLR1, TLR5, TRLR6, TRAF3, TLR8, TLR4, MYD88, and IRAK3. In some embodiments, the transcriptomic signature comprises VWF. In embodiments, the any one or more genes comprise any one or more of CLU, ITGA2B, MGLL, PROS1, GP1BA, GP9, SPARC, and YES1.

In some embodiments, the transcriptomic signature is predictive of a severe form of a disease or a condition in a subject, such as an inflammatory bowel disease (IBD). In some embodiments, the IBD comprises Crohn's disease (CD) or ulcerative colitis (UC). In some embodiments, the subtype characterized by an increase or a decrease in serological markers that display an immune reactivity to a microbial antigen. In some embodiments, the serological markers comprise antineutrophil cytoplasmic antibodies (ANCA), antibodies (IgG) against the yeast Saccharomyces cerevisiae (ASCA), or a combination thereof. In some embodiments, subtype is associated with an increase or a decrease in a quartile sum score (QSS) of such serological markers. A quartile sum score as disclosed herein may be calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699, which is hereby incorporated by reference in its entirety. In some embodiments, the subtype is associated with disease in a particular tissue of the GI tract, such as the large intestine or the small intestine. In some embodiments, the particular tissue comprises the colon, ileum, or ileocolonic region of the intestine, or a combination thereof.

In some embodiments, gene expression profiling may be used as a research tool to identify new markers for diagnosis and/or classification of an IBD disease or condition, to monitor the effect of drugs or candidate drugs on biological samples and/or patients, to uncover new pathways for IBD treatment, or any combination thereof.

The expression profile of a transcriptomic signature in a subject may be determined by analyzing genetic material obtained from a subject. The subject may be human. In some embodiments, the genetic material is obtained from a subject having an inflammatory disease, such as inflammatory bowel disease, or specifically, Crohn's Disease. Although the methods described herein are generally referenced for use with Crohn's Disease patients, in some cases the methods and transcriptomic signatures are applicable to other inflammatory diseases, including, ulcerative colitis.

In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., from the intestinal track of the subject. Isolating components of fluid or tissue samples (e.g., cells or RNA or DNA) may be accomplished using a variety of techniques. After the sample is obtained, it may be further processed to enrich for or purify genomic material. Any of the foregoing samples or techniques may also be used to obtain genetic material for detecting for the presence of one or more polymorphisms comprising rs763361 or a proxy polymorphism in linkage disequilibrium therewith as determined with an r² of at least 0.85, or a combination thereof.

In some embodiments, the expression level of a biomarker in a sample from a subject is compared to a reference expression level (or reference expression profile). In some cases, the reference expression level is from a subject that does not comprise IBD. In some cases, the reference expression level is from a subject that does not comprises a severe form of IBD.

TABLE 1
Differentially Expressed Genes (DGE) that contain a fold change ≥2.0

| | | | Geom | | | | | | |
|---|---|---|---|---|---|---|---|---|---|
| | | Geom | mean of | | | | | | |
| | | mean of | intensities | | | | | | |
| Parametric | | intensities | PAF- | Fold- | | Entrez | | | |
| p-value | FDR | Cdmono | Cdmono | change | UniqueID | ID | Accession | UGCluster | Ensembl |
| <1e−07 | <1e−07 | 225.46 | 74.9 | 3.01 | ODC1 | 4953 | NM_001287188 | Hs.467701 | ENSG00000115758 |
| <1e−07 | <1e−07 | 234.01 | 77.19 | 3.03 | MTURN | 222166 | NM_152793 | Hs.200100 | ENSG00000180354 |
| <1e−07 | <1e−07 | 139.21 | 53.68 | 2.59 | PCGF5 | 84333 | NM_001256549 | Hs.500512 | ENSG00000180628 |
| <1e−07 | <1e−07 | 648.43 | 300.89 | 2.16 | ITGB1 | 3688 | NM_002211 | Hs.643813 | ENSG00000150093 |
| <1e−07 | <1e−07 | 69.6 | 20.5 | 3.39 | PBX1 | 5087 | NM_001204961 | Hs.557097 | ENSG00000185630 |
| <1e−07 | <1e−07 | 456.51 | 152.27 | 3 | H2AC6 | | | | ENSG00000180573 |
| <1e−07 | <1e−07 | 288.87 | 78.28 | 3.69 | PDLIM1 | 9124 | NM_020992 | Hs.368525 | ENSG00000107438 |
| <1e−07 | <1e−07 | 436.15 | 134.19 | 3.25 | GRAP2 | 9402 | NM_001291824 | Hs.517499 | ENSG00000100351 |
| <1e−07 | <1e−07 | 260.27 | 125.55 | 2.07 | TLK1 | 9874 | NM_001136554 | Hs.744917 | ENSG00000198586 |
| <1e−07 | <1e−07 | 194.9 | 65.35 | 2.98 | RGS10 | 6001 | NM_001005339 | Hs.501200 | ENSG00000148908 |
| <1e−07 | <1e−07 | 402.3 | 108.12 | 3.72 | SH3BGRL2 | 83699 | NM_031469 | Hs.302772 | ENSG00000198478 |
| <1e−07 | <1e−07 | 501.03 | 174.85 | 2.87 | KIF2A | 3796 | NM_001098511 | Hs.558351 | ENSG00000068796 |
| <1e−07 | <1e−07 | 290.41 | 106.23 | 2.73 | RNF11 | 26994 | NM_014372 | Hs.309641 | ENSG00000123091 |
| <1e−07 | <1e−07 | 493.87 | 185.42 | 2.66 | MAX | 4149 | NM_001271068 | Hs.285354 | ENSG00000125952 |
| <1e−07 | <1e−07 | 25.55 | 7.06 | 3.62 | VEPH1 | 79674 | NM_001167911 | Hs.658046 | ENSG00000197415 |
| <1e−07 | <1e−07 | 51.48 | 16.62 | 3.1 | TTC7B | 145567 | NM_001010854 | Hs.655697 | ENSG00000165914 |
| <1e−07 | <1e−07 | 657.3 | 322.21 | 2.04 | NAP1L1 | 4673 | NM_001307924 | Hs.524599 | ENSG00000187109 |
| <1e−07 | <1e−07 | 198.24 | 90.74 | 2.18 | CMPK1 | 51727 | NM_001136140 | Hs.731647 | ENSG00000162368 |
| <1e−07 | <1e−07 | 102.48 | 30.16 | 3.4 | LGALSL | 29094 | NM_014181 | Hs.372208 | ENSG00000119862 |
| <1e−07 | <1e−07 | 55.18 | 16.9 | 3.27 | MEIS1 | 4211 | NM_002398 | Hs.526754 | ENSG00000143995 |
| <1e−07 | <1e−07 | 191.13 | 53.52 | 3.57 | LTBP1 | 4052 | NM_000627 | Hs.619315 | ENSG00000049323 |
| <1e−07 | <1e−07 | 258.72 | 76.2 | 3.4 | CTTN | 2017 | NM_001184740 | Hs.596164 | ENSG00000085733 |
| <1e−07 | <1e−07 | 56.27 | 15.52 | 3.63 | CDC14B | 8555 | NM_001077181 | Hs.40582 | ENSG00000081377 |
| <1e−07 | <1e−07 | 1511.38 | 381.79 | 3.96 | CCL5 | 6352 | NM_001278736 | Hs.514821 | ENSG00000271503 |
| <1e−07 | <1e−07 | 52.46 | 17.79 | 2.95 | TSPO | 1.01E+08 | NR_038410 | Hs.718477 | ENSG00000265148 |
| | | | | | AP1-AS1 | | | | |
| <1e−07 | <1e−07 | 504.84 | 173.22 | 2.91 | PTGS1 | 5742 | NM_000962 | Hs.201978 | ENSG00000095303 |
| <1e−07 | <1e−07 | 114.93 | 40.11 | 2.87 | CCDC92 | 80212 | NM_001304957 | Hs.114111 | ENSG00000119242 |
| <1e−07 | <1e−07 | 26.78 | 7.02 | 3.81 | TRAPPC3L | 1E+08 | NM_001139444 | Hs.134795 | ENSG00000173626 |
| <1e−07 | <1e−07 | 28.3 | 8.64 | 3.28 | RGS6 | 9628 | NM_001204416 | Hs.509872 | ENSG00000182732 |
| <1e−07 | <1e−07 | 108.52 | 31.65 | 3.43 | CTDSPL | 10217 | NM_001008392 | Hs.475963 | ENSG00000144677 |
| <1e−07 | <1e−07 | 127.5 | 37.33 | 3.42 | GFI1B | 8328 | NM_001135031 | Hs.553160 | ENSG00000165702 |
| <1e−07 | <1e−07 | 26.77 | 8.32 | 3.22 | ATP9A | 10079 | NM_006045 | Hs.649234 | ENSG00000054793 |
| <1e−07 | <1e−07 | 139.72 | 38.28 | 3.65 | ENDOD1 | 23052 | NM_015036 | Hs.167115 | ENSG00000149218 |
| <1e−07 | <1e−07 | 104.95 | 28.87 | 3.64 | SELP | 6403 | NM_003005 | Hs.73800 | ENSG00000174175 |
| <1e−07 | <1e−07 | 711.36 | 233.37 | 3.05 | OST4 | 1E+08 | NM_001134693 | Hs.502948 | ENSG00000228474 |
| <1e−07 | <1e−07 | 634.26 | 212.68 | 2.98 | PRKAR2B | 5577 | NM_002736 | Hs.433068 | ENSG00000005249 |
| <1e−07 | <1e−07 | 198.26 | 86.66 | 2.29 | MOB1B | 92597 | NM_001244766 | Hs.691454 | ENSG00000173542 |
| <1e−07 | <1e−07 | 312.59 | 152.53 | 2.05 | MPP1 | 4354 | NM_001166460 | Hs.496984 | ENSG00000130830 |
| <1e−07 | <1e−07 | 181.22 | 53.45 | 3.39 | FAXDC2 | 10826 | NM_016348 | Hs.519694 | ENSG00000170271 |
| <1e−07 | <1e−07 | 34.87 | 10.35 | 3.37 | PARD3 | 56288 | NM_001184785 | Hs.131489 | ENSG00000148498 |
| <1e−07 | <1e−07 | 68.31 | 27.4 | 2.49 | SEPTIN11 | | | | ENSG00000138758 |
| <1e−07 | <1e−07 | 41.14 | 12.1 | 3.4 | EGF | 1950 | NM_001178130 | Hs.419815 | ENSG00000138798 |
| <1e−07 | <1e−07 | 309.01 | 88.44 | 3.49 | C2orf88 | 84281 | NM_001042519 | Hs.389311 | ENSG00000187699 |
| <1e−07 | <1e−07 | 181.68 | 52.06 | 3.49 | PDE5A | 8654 | NM_001083 | Hs.647971 | ENSG00000138735 |
| <1e−07 | <1e−07 | 739.62 | 223.3 | 3.31 | SPARC | 6678 | NM_001309443 | Hs.111779 | ENSG00000113140 |
| <1e−07 | <1e−07 | 250.44 | 80.62 | 3.11 | CD226 | 10666 | NM_001303618 | Hs.660130 | ENSG00000150637 |
| <1e−07 | <1e−07 | 997.18 | 328.35 | 3.04 | CAVIN2 | 8436 | NM_004657 | Hs.26530 | ENSG00000168497 |
| <1e−07 | <1e−07 | 180.53 | 55.65 | 3.24 | MMD | 23531 | NM_012329 | Hs.463483 | ENSG00000108960 |
| <1e−07 | <1e−07 | 524.63 | 204.32 | 2.57 | LIMS1 | 3987 | NM_001193482 | Hs.597715 | ENSG00000169756 |
| <1e−07 | <1e−07 | 196.67 | 53.7 | 3.66 | TREML1 | 340205 | NM_001271807 | Hs.117331 | ENSG00000161911 |
| <1e−07 | <1e−07 | 118.11 | 36.29 | 3.25 | ACRBP | 84519 | NM_032489 | Hs.123239 | ENSG00000111644 |
| <1e−07 | <1e−07 | 61.01 | 18.48 | 3.3 | ARHGAP6 | 395 | NM_001174 | Hs.435291 | ENSG00000047648 |
| <1e−07 | <1e−07 | 231.2 | 67.3 | 3.44 | GNG11 | 2791 | NM_004126 | Hs.83381 | ENSG00000127920 |
| <1e−07 | <1e−07 | 151.35 | 47.78 | 3.17 | DNM3 | 26052 | NM_001136127 | Hs.654775 | ENSG00000197959 |
| <1e−07 | <1e−07 | 169.32 | 53.07 | 3.19 | MYLK | 4638 | NM_001321309 | Hs.477375 | ENSG00000065534 |
| <1e−07 | <1e−07 | 218.55 | 78.29 | 2.79 | PGRMC1 | 10857 | NM_001282621 | Hs.90061 | ENSG00000101856 |
| <1e−07 | <1e−07 | 27.15 | 7.75 | 3.5 | VIL 1 | 7429 | NM_007127 | Hs.654595 | ENSG00000127831 |
| <1e−07 | <1e−07 | 49.36 | 14.18 | 3.48 | TC2N | 123036 | NM_001128595 | Hs.510262 | ENSG00000165929 |
| <1e−07 | <1e−07 | 67.34 | 28.3 | 2.38 | PTK2 | 5747 | NM_001199649 | Hs.395482 | ENSG00000169398 |
| <1e−07 | <1e−07 | 793.47 | 233.6 | 3.4 | MPIG6B | 80739 | NM_025260 | Hs.247879 | ENSG00000204420 |
| <1e−07 | <1e−07 | 144.48 | 60.52 | 2.39 | PRDX6 | 9588 | NM_004905 | Hs.120 | ENSG00000117592 |
| <1e−07 | <1e−07 | 144.37 | 52.29 | 2.76 | DAB2 | 1601 | NM_001244871 | Hs.696631 | ENSG00000153071 |
| <1e−07 | <1e−07 | 55.51 | 16.25 | 3.42 | PLA2G12A | 81579 | NM_030821 | Hs.389452 | ENSG00000123739 |
| <1e−07 | <1e−07 | 52.44 | 16.06 | 3.27 | H2BC11 | 8970 | | | |
| <1e−07 | <1e−07 | 170.95 | 81.01 | 2.11 | LRBA | 987 | NM_001199282 | Hs.480938 | ENSG00000198589 |
| <1e−07 | <1e−07 | 112.29 | 36.03 | 3.12 | TAL1 | 6886 | NM_001287347 | Hs.705618 | ENSG00000162367 |
| <1e−07 | <1e−07 | 156.46 | 50.92 | 3.07 | ARHGEF12 | 23365 | NM_001198665 | Hs.24598 | ENSG00000196914 |
| <1e−07 | <1e−07 | 480.2 | 164.97 | 2.91 | RGS18 | 64407 | NM_130782 | Hs.440890 | ENSG00000150681 |
| <1e−07 | <1e−07 | 202 | 54.54 | 3.7 | MFAP3L | 9848 | NM_001009554 | Hs.593942 | ENSG00000198948 |
| <1e−07 | <1e−07 | 302.62 | 93.28 | 3.24 | CXCR2P1 | 3580 | NR_002712 | Hs.647858 | |
| <1e−07 | <1e−07 | 88.94 | 28.4 | 3.13 | ELOVL7 | 79993 | NM_001104558 | Hs.274256 | ENSG00000164181 |
| <1e−07 | <1e−07 | 2357.19 | 783.27 | 3.01 | TUBB1 | 81027 | NM_030773 | Hs.303023 | ENSG00000101162 |
| <1e−07 | <1e−07 | 146.31 | 61.18 | 2.39 | ANKRD28 | 23243 | NM_001195098 | Hs.335239 | ENSG00000206560 |
| <1e−07 | <1e−07 | 75.77 | 28.89 | 2.62 | PRUNE1 | 58497 | NM_001303229 | Hs.78524 | ENSG00000143363 |
| <1e−07 | <1e−07 | 59. | 16.88 | 3.5 | GUCY1A1 | 2982 | NM_000856 | Hs.24258 | ENSG00000164116 |
| <1e−07 | <1e−07 | 188.85 | 94.42 | 2 | MAPRE2 | 10982 | NM_001143826 | Hs.532824 | ENSG00000166974 |
| <1e−07 | <1e−07 | 71.59 | 21.76 | 3.29 | CPNE5 | 57699 | NM_001314017 | Hs.372129 | ENSG00000124772 |
| <1e−07 | <1e−07 | 203.6 | 70.56 | 2.89 | STON2 | 85439 | NM_001256430 | Hs.14248 | ENSG00000140022 |
| <1e−07 | <1e−07 | 71.21 | 21.53 | 3.31 | GUCY1B1 | 2983 | NM_000857 | Hs.77890 | ENSG00000061918 |
| <1e−07 | <1e−07 | 199.5 | 93.7 | 2.13 | ARHGAP21 | 57584 | NM_020824 | Hs.524195 | ENSG00000107863 |
| <1e−07 | <1e−07 | 176.94 | 78.6 | 2.25 | ZNF185 | 7739 | NM_001178106 | Hs.16622 | ENSG00000147394 |
| <1e−07 | <1e−07 | 101.9 | 29.08 | 3.5 | BEX3 | 27018 | NM_001282674 | Hs.448588 | ENSG00000166681 |
| <1e−07 | <1e−07 | 49.14 | 14.77 | 3.33 | SVIP | 258010 | NM_001320340 | Hs.349096 | ENSG00000198168 |
| <1e−07 | <1e−07 | 29.66 | 10.23 | 2.9 | MED12L | 116931 | NM_053002 | Hs.744234 | ENSG00000144893 |
| <1e−07 | <1e−07 | 260.94 | 108.07 | 2.41 | TUBA4A | 7277 | NM_001278552 | Hs.75318 | ENSG00000127824 |
| <1e−07 | <1e−07 | 103.06 | 28.78 | 3.58 | ABLIM3 | 22885 | NM_001301015 | Hs.49688 | ENSG00000173210 |
| <1e−07 | <1e−07 | 102.92 | 29.04 | 3.54 | BEND2 | 139105 | NM_001184767 | Hs.403802 | ENSG00000177324 |
| <1e−07 | <1e−07 | 48.38 | 14.44 | 3.35 | WHAMMP3 | 339005 | NR_003521 | Hs.212670 | |
| <1e−07 | <1e−07 | 75.13 | 22.06 | 3.41 | PEAR1 | 375033 | NM_001080471 | Hs.142003 | ENSG00000187800 |
| <1e−07 | <1e−07 | 696.69 | 341 | 2.04 | RAP1B | 5908 | NM_001010942 | Hs.369920 | ENSG00000127314 |
| <1e−07 | <1e−07 | 98.09 | 34.28 | 2.86 | ASAP2 | 8853 | NM_001135191 | Hs.555902 | ENSG00000151693 |
| <1e−07 | <1e−07 | 30.52 | 8.81 | 3.46 | RAB6B | 51560 | NM_016577 | Hs.715344 | ENSG00000154917 |
| <1e−07 | <1e−07 | 85.74 | 23.86 | 3.59 | PCSK6 | 5046 | NM_001291309 | Hs.498494 | ENSG00000140479 |
| <1e−07 | <1e−07 | 53.69 | 21.81 | 2.46 | MLH3 | 27030 | NM_001040108 | Hs.436650 | ENSG00000119684 |
| <1e−07 | <1e−07 | 43.3 | 13.04 | 3.32 | BANK1 | 55024 | NM_001083907 | Hs.480400 | ENSG00000153064 |
| <1e−07 | <1e−07 | 647.05 | 172 | 3.76 | PF4 | 5196 | NM_002619 | Hs.81564 | ENSG00000163737 |
| <1e−07 | <1e−07 | 247.62 | 95.51 | 2.59 | NCK2 | 8440 | NM_001004720 | Hs.529244 | ENSG00000071051 |
| <1e−07 | <1e−07 | 233.6 | 94.98 | 2.46 | TSC22D1 | 8848 | NM_001243797 | Hs.436383 | ENSG00000102804 |
| <1e−07 | <1e−07 | 15.32 | 5.9 | 2.6 | C12orf76 | 400073 | NM_207435 | Hs.44817 | ENSG00000174456 |
| <1e−07 | <1e−07 | 126.71 | 47.6 | 2.66 | ARHGAP18 | 93663 | NM_033515 | Hs.486458 | ENSG00000146376 |
| <1e−07 | <1e−07 | 1252.05 | 446.29 | 2.81 | F13A1 | 2162 | NM_000129 | Hs.335513 | ENSG00000124491 |
| <1e−07 | <1e−07 | 160.72 | 62.6 | 2.57 | STOM | 2040 | NM_001270526 | Hs.253903 | ENSG00000148175 |
| <1e−07 | <1e−07 | 80.83 | 22.89 | 3.53 | CA2 | 760 | NM_000067 | Hs.155097 | ENSG00000104267 |
| <1e−07 | <1e−07 | 136.34 | 55.53 | 2.46 | CNST | 163882 | NM_001139459 | Hs.368353 | ENSG00000162852 |
| <1e−07 | <1e−07 | 910.88 | 397.49 | 2.29 | VCL | 7414 | NM_003373 | Hs.643896 | ENSG00000035403 |
| <1e−07 | <1e−07 | 99.12 | 29.88 | 3.32 | PKHD1L1 | 93035 | NM_177531 | Hs.170128 | ENSG00000205038 |
| <1e−07 | <1e−07 | 21.41 | 9.44 | 2.27 | AL365361.1 | | | | ENSG00000259834 |
| <1e−07 | <1e−07 | 222.61 | 103.44 | 2.15 | RAB11A | 8766 | NM_001206836 | Hs.321541 | ENSG00000103769 |
| <1e−07 | <1e−07 | 223.36 | 105.04 | 2.13 | EIF2AK1 | 27102 | NM_001134335 | Hs.520205 | ENSG00000086232 |
| <1e−07 | <1e−07 | 96.44 | 32.29 | 2.99 | TPM1 | 7168 | NM_000366 | Hs.133892 | ENSG00000140416 |
| <1e−07 | <1e−07 | 23.8 | 7.77 | 3.06 | AC114752.2 | | | | |
| <1e−07 | <1e−07 | 202.96 | 63.17 | 3.21 | GNAZ | 2781 | NM_002073 | Hs.584760 | ENSG00000128266 |
| <1e−07 | <1e−07 | 63.96 | 20.08 | 3.19 | IGF2BP3 | 10643 | NM_006547 | Hs.700696 | ENSG00000136231 |
| <1e−07 | <1e−07 | 69.34 | 20.97 | 3.31 | TMEM40 | 55287 | NM_001284406 | Hs.475502 | ENSG00000088726 |
| <1e−07 | <1e−07 | 257.25 | 72.38 | 3.55 | RAB27B | 5874 | NM_004163 | Hs.25318 | ENSG00000041353 |
| <1e−07 | <1e−07 | 599.89 | 154.93 | 3.87 | MYL9 | 10398 | NM_006097 | Hs.504687 | ENSG00000101335 |
| <1e−07 | <1e−07 | 340.8 | 105.49 | 3.23 | TRIM58 | 25893 | NM_015431 | Hs.269151 | ENSG00000162722 |
| <1e−07 | <1e−07 | 57.17 | 24.38 | 2.34 | PRKACB | 5567 | NM_001242857 | Hs.487325 | ENSG00000142875 |
| <1e−07 | <1e−07 | 27.88 | 9.32 | 2.99 | NCKAP1 | 10787 | NM_013436 | Hs.603732 | ENSG00000061676 |
| <1e−07 | <1e−07 | 33.29 | 10.41 | 3.2 | XK | 7504 | NM_021083 | Hs.78919 | ENSG00000047597 |
| <1e−07 | <1e−07 | 114.08 | 44.66 | 2.55 | NT5C3A | 51251 | NM_001002009 | Hs.487933 | ENSG00000122643 |
| <1e−07 | <1e−07 | 33.29 | 9.36 | 3.55 | LCN2 | 3934 | NM_005564 | Hs.204238 | ENSG00000148346 |
| <1e−07 | <1e−07 | 45.21 | 14.08 | 3.2 | GP6 | 51206 | NM_001083899 | Hs.661752 | ENSG00000088053 |
| <1e−07 | <1e−07 | 50.43 | 15.69 | 3.21 | RHOBTB1 | 9886 | NM_001242359 | Hs.737374 | ENSG00000072422 |
| <1e−07 | <1e−07 | 42.51 | 17.4 | 2.44 | MCUR1 | 63933 | NM_001031713 | Hs.214043 | ENSG00000050393 |
| <1e−07 | <1e−07 | 32.17 | 13.05 | 2.47 | SIAE | 54414 | NM_001199922 | Hs.10056 | ENSG00000110013 |
| <1e−07 | <1e−07 | 40.64 | 13.35 | 3.04 | H2AC11 | 8969 | | | |
| <1e−07 | <1e−07 | 3432.04 | 1274.28 | 2.69 | NRGN | 4900 | NM_001126181 | Hs.524116 | ENSG00000154146 |
| <1e−07 | <1e−07 | 14 | 6.04 | 2.32 | ACTR3B | 57180 | NM_001040135 | Hs.647117 | ENSG00000133627 |
| <1e−07 | <1e−07 | 27.14 | 8.24 | 3.3 | SPX | 80763 | NM_030572 | Hs.130692 | ENSG00000134548 |
| <1e−07 | <1e−07 | 65.98 | 21.6 | 3.05 | SLA2 | 84174 | NM_032214 | Hs.713578 | ENSG00000101082 |
| <1e−07 | <1e−07 | 35.86 | 10.41 | 3.44 | WDR11- | 283089 | NR_033850 | Hs.568750 | ENSG00000227165 |
| <1e−07 | <1e−07 | 83.15 | 26.32 | 3.16 | ITGB5 | 3693 | NM_001354764 | Hs.13155 | ENSG00000082781 |
| <1e−07 | <1e−07 | 98.39 | 30.24 | 3.25 | ALOX12 | 239 | NM_000697 | Hs.654431 | ENSG00000108839 |
| <1e−07 | <1e−07 | 217.46 | 58.06 | 3.75 | H3C10 | 8357 | | | |
| <1e−07 | <1e−07 | 87.31 | 41.62 | 2.1 | MINDY1 | 55793 | NM_001040217 | Hs.743952 | ENSG00000143409 |
| <1e−07 | <1e−07 | 18.01 | 6.52 | 2.76 | AL731557.1 | | | | |
| <1e−07 | <1e−07 | 675.57 | 240.79 | 2.81 | CLU | 1191 | NM_001831 | Hs.436657 | ENSG00000120885 |
| <1e−07 | <1e−07 | 35.77 | 11.31 | 3.16 | PCYT1B | 9468 | NM_001163264 | Hs.660708 | ENSG00000102230 |
| <1e−07 | <1e−07 | 28.35 | 12.15 | 2.33 | SCFD2 | 152579 | NM_152540 | Hs.302287 | ENSG00000184178 |
| <1e−07 | <1e−07 | 38.12 | 11.69 | 3.26 | P2RY12 | 64805 | NM_022788 | Hs.591281 | ENSG00000169313 |
| <1e−07 | <1e−07 | 319.35 | 96.37 | 3.31 | ITGB3 | 3690 | NM_000212 | Hs.218040 | ENSG00000259207 |
| <1e−07 | <1e−07 | 26.08 | 11.49 | 2.27 | TTC33 | 23548 | NM_012382 | Hs.348915 | ENSG00000113638 |
| <1e−07 | <1e−07 | 38.86 | 11.6 | 3.35 | FHL1 | 2273 | NM_001159699 | Hs.435369 | ENSG00000022267 |
| <1e−07 | <1e−07 | 242.33 | 73.17 | 3.31 | GP1BA | 2811 | NM_000173 | Hs.1472 | ENSG00000185245 |
| <1e−07 | <1e−07 | 377.76 | 181.49 | 2.08 | PIP4K2A | 5305 | NM_001330062 | Hs.57079 | ENSG00000150867 |
| <1e−07 | <1e−07 | 85.82 | 23.1 | 3.72 | ANK1 | 286 | NM_000037 | Hs.654438 | ENSG00000029534 |
| <1e−07 | <1e−07 | 47.24 | 14.23 | 3.32 | F2R | 2149 | NM_001311313 | Hs.482562 | ENSG00000181104 |
| <1e−07 | <1e−07 | 221.56 | 63.61 | 3.48 | DMTN | 2039 | NM_001114135 | Hs.106124 | ENSG00000158856 |
| <1e−07 | <1e−07 | 91.4 | 45.53 | 2.01 | NUTF2 | 10204 | NM_001322038 | Hs.356630 | ENSG00000102898 |
| <1e−07 | <1e−07 | 20.21 | 7.22 | 2.8 | ZNF367 | 195828 | NM_153695 | Hs.494557 | ENSG00000165244 |
| <1e−07 | <1e−07 | 49.7 | 15.15 | 3.28 | CMTM5 | 116173 | NM_001037288 | Hs.99272 | ENSG00000166091 |
| <1e−07 | <1e−07 | 61.21 | 18.05 | 3.39 | CALD1 | 800 | NM_004342 | Hs.490203 | ENSG00000122786 |
| <1e−07 | <1e−07 | 48.41 | 13.36 | 3.62 | WHAMMP2 | 440253 | NR_026589 | Hs.558967 | |
| <1e−07 | <1e−07 | 21.7 | 7.63 | 2.84 | AP001636.3 | | | | |
| <1e−07 | <1e−07 | 79.51 | 24.01 | 3.31 | LINC00989 | 1.01E+08 | NR_038826 | Hs.507664 | ENSG00000250334 |
| <1e−07 | <1e−07 | 119.65 | 55.5 | 2.16 | AGPAT1 | 10554 | NM_006411 | Hs.409230 | ENSG00000204310 |
| <1e−07 | <1e−07 | 21.26 | 7.73 | 2.75 | SENCR | 1.01E+08 | NR_038908 | Hs.657715 | ENSG00000254703 |
| <1e−07 | <1e−07 | 22.12 | 7.98 | 2.77 | MSANTD3 | 91283 | NM_001198805 | Hs.530272 | ENSG00000066697 |
| <1e−07 | <1e−07 | 1662.12 | 562.73 | 2.95 | PPBP | 5473 | NM_002704 | Hs.2164 | ENSG00000163736 |
| <1e−07 | <1e−07 | 54.33 | 18.38 | 2.96 | Clorf198 | 84886 | NM_001136494 | Hs.520494 | ENSG00000119280 |
| <1e−07 | <1e−07 | 98.83 | 29.51 | 3.35 | PTCRA | 171558 | NM_001243168 | Hs.169002 | ENSG00000171611 |
| <1e−07 | <1e−07 | 32.08 | 10.16 | 3.16 | WASF3 | 10810 | NM_001291965 | Hs.618732 | ENSG00000132970 |
| <1e−07 | <1e−07 | 17.58 | 6.26 | 2.81 | C12orf75 | 387882 | NM_001145199 | Hs.368938 | ENSG00000235162 |
| <1e−07 | <1e−07 | 37.13 | 12.76 | 2.91 | TNFSF4 | 7292 | NM_001297562 | Hs.181097 | ENSG00000117586 |
| <1e−07 | <1e−07 | 73.85 | 23.79 | 3.1 | BMP6 | 654 | NM_001718 | Hs.285671 | ENSG00000153162 |
| <1e−07 | <1e−07 | 23.13 | 7.07 | 3.27 | AL445426.1 | | | | |
| <1e−07 | <1e−07 | 134.7 | 51.7 | 2.6 | EHD3 | 30845 | NM_014600 | Hs.368808 | ENSG00000013016 |
| <1e−07 | <1e−07 | 85.07 | 37.59 | 2.26 | NDUFAF3 | 25915 | NM_199069 | Hs.31387 | ENSG00000178057 |
| <1e−07 | <1e−07 | 16.61 | 6.99 | 2.38 | RTCA- | 1.01E+08 | NR_110434 | Hs.192268 | |
| | | | | | AS1 | | | | |
| <1e−07 | <1e−07 | 72.85 | 23.23 | 3.14 | ESAM | 90952 | NM_138961 | Hs.173840 | ENSG00000149564 |
| <1e−07 | <1e−07 | 19.67 | 7.26 | 2.71 | LINC01011 | 401232 | NM_207495 | Hs.720158 | |
| <1e−07 | <1e−07 | 18.8 | 6.53 | 2.88 | GIPC3 | 126326 | NM_133261 | Hs.266873 | ENSG00000179855 |
| <1e−07 | <1e−07 | 83.96 | 33.59 | 2.5 | LDLRAP1 | 26119 | NM_015627 | Hs.590911 | ENSG00000157978 |
| <1e−07 | <1e−07 | 21.66 | 7.9 | 2.74 | Z82206.1 | | | | |
| <1e−07 | <1e−07 | 58.99 | 25.15 | 2.35 | TNIK | 23043 | NM_001161560 | Hs.34024 | ENSG00000154310 |
| <1e−07 | <1e−07 | 52.54 | 26.14 | 2.01 | NLK | 51701 | NM_016231 | Hs.208759 | ENSG00000087095 |
| <1e−07 | <1e−07 | 39.43 | 14.2 | 2.78 | TFPI | 7035 | NM_001032281 | Hs.516578 | ENSG00000003436 |
| <1e−07 | <1e−07 | 48.18 | 15.23 | 3.16 | CLEC1B | 51266 | NM_001099431 | Hs.409794 | ENSG00000165682 |
| <1e−07 | <1e−07 | 13.62 | 5.9 | 2.31 | SLC8A3 | 6547 | NM_001130417 | Hs.337696 | ENSG00000100678 |
| <1e−07 | <1e−07 | 89.66 | 40.95 | 2.19 | RAB37 | 326624 | NM_001006637 | Hs.351413 | ENSG00000172794 |
| <1e−07 | <1e−07 | 29.17 | 13.19 | 2.21 | LYPLAL1 | 127018 | NM_001300769 | Hs.657617 | ENSG00000143353 |
| <1e−07 | <1e−07 | 23.29 | 10.81 | 2.16 | TWSG1 | 57045 | NM_020648 | Hs.514685 | ENSG00000128791 |
| <1e−07 | <1e−07 | 17.68 | 6.72 | 2.63 | ZNF385D | 79750 | NM_024697 | Hs.21026 | ENSG00000151789 |
| <1e−07 | <1e−07 | 74.98 | 21.2 | 3.54 | ENKUR | 219670 | NM_001270383 | Hs.534486 | ENSG00000151023 |
| <1e−07 | <1e−07 | 32.96 | 10.33 | 3.19 | TMCC2 | 9911 | NM_001242925 | Hs.6360 | ENSG00000133069 |
| <1e−07 | <1e−07 | 14.46 | 5.92 | 2.44 | TDRP | 157695 | NM_001256113 | Hs.289293 | ENSG00000180190 |
| <1e−07 | <1e−07 | 45.81 | 19.01 | 2.4 | SAVI | 60485 | NM_021818 | Hs.642842 | ENSG00000151748 |
| <1e−07 | <1e−07 | 24.08 | 8.6 | 2.8 | LPAR5 | 57121 | NM_001142961 | Hs.155538 | ENSG00000184574 |
| <1e−07 | <1e−07 | 20.62 | 7.86 | 2.62 | GGTA1P | 2681 | NR_003191 | Hs.97469 | ENSG00000204136 |
| <1e−07 | <1e−07 | 27.33 | 10.83 | 2.52 | NFATC2 | 4773 | NM_001136021 | Hs.744148 | ENSG00000101096 |
| <1e−07 | <1e−07 | 21.07 | 7.33 | 2.88 | AC147651.1 | | | | |
| <1e−07 | <1e−07 | 57.36 | 16.68 | 3.44 | SPTB | 6710 | NM_000347 | Hs.417303 | ENSG00000070182 |
| <1e−07 | <1e−07 | 15.57 | 6.12 | 2.55 | GNAO1 | 2775 | NM_020988 | Hs.644524 | ENSG00000087258 |
| <1e−07 | <1e−07 | 63.27 | 15.81 | 4 | CXCL5 | 6374 | NM_002994 | Hs.89714 | ENSG00000163735 |
| <1e−07 | <1e−07 | 52.27 | 19.54 | 2.68 | H2BC7 | 8343 | | | |
| <1e−07 | <1e−07 | 20.03 | 7.4 | 2.71 | PDE3A | 5139 | NM_000921 | Hs.386791 | ENSG00000172572 |
| <1e−07 | <1e−07 | 94.04 | 33.28 | 2.83 | PITPNM2 | 57605 | NM_001300801 | Hs.272759 | ENSG00000090975 |
| <1e−07 | <1e−07 | 48.21 | 18.82 | 2.56 | SSX2IP | 117178 | NM_001166293 | Hs.22587 | ENSG00000117155 |
| <1e−07 | <1e−07 | 38.53 | 14.58 | 2.64 | PDGFA | 5154 | NM_002607 | Hs.535898 | ENSG00000197461 |
| <1e−07 | <1e−07 | 134.3 | 54.86 | 2.45 | BCL2L1 | 598 | NM_001191 | Hs.516966 | ENSG00000171552 |
| <1e−07 | <1e−07 | 61.61 | 18.43 | 3.34 | SNCA | 6622 | NM_000345 | Hs.21374 | ENSG00000145335 |
| <1e−07 | <1e−07 | 67.33 | 30.84 | 2.18 | SMIM3 | 85027 | NM_032947 | Hs.29444 | ENSG00000256235 |
| <1e−07 | <1e−07 | 46.2 | 18.86 | 2.45 | ISCA1 | 81689 | NM_030940 | Hs.449291 | ENSG00000135070 |
| <1e−07 | <1e−07 | 3011.7 | 1489.98 | 2.02 | TMSB4X | 7114 | NM_021109 | Hs.437277 | ENSG00000205542 |
| <1e−07 | <1e−07 | 42.53 | 14.58 | 2.92 | H2BC9 | 8345 | | | |
| <1e−07 | <1e−07 | 21.59 | 8.86 | 2.44 | CCDC7 | 79741 | NM_001026383 | Hs.585464 | ENSG00000216937 |
| <1e−07 | <1e−07 | 78.26 | 29.11 | 2.69 | SMOX | 54498 | NM_001270691 | Hs.433337 | ENSG00000088826 |
| <1e−07 | <1e−07 | 15.77 | 6.22 | 2.53 | SH3TC2 | 79628 | NM_024577 | Hs.483784 | ENSG00000169247 |
| <1e−07 | <1e−07 | 17.65 | 6.63 | 2.66 | NT5M | 56953 | NM_020201 | Hs.513977 | ENSG00000205309 |
| <1e−07 | <1e−07 | 88.2 | 24.75 | 3.56 | JAM3 | 83700 | NM_001205329 | Hs.150718 | ENSG00000166086 |
| <1e−07 | <1e−07 | 42.26 | 13.4 | 3.15 | MPL | 4352 | NM_005373 | Hs.82906 | ENSG00000117400 |
| <1e−07 | <1e−07 | 34.05 | 15.82 | 2.15 | SLC10A3 | 8273 | NM_001142391 | Hs.522826 | ENSG00000126903 |
| <1e−07 | <1e−07 | 10.89 | 5.33 | 2.04 | PKIG | 11142 | NM_001281444 | Hs.472831 | ENSG00000168734 |
| <1e−07 | <1e−07 | 72.8 | 24.6 | 2.96 | ABCC4 | 10257 | NM_001105515 | Hs.508423 | ENSG00000125257 |
| <1e−07 | <1e−07 | 13.01 | 6.1 | 2.13 | TMSB4XP8 | 7117 | NM_183049 | | |
| <1e−07 | <1e−07 | 85.89 | 27.64 | 3.11 | MMRN1 | 22915 | NM_007351 | Hs.268107 | ENSG00000138722 |
| <1e−07 | <1e−07 | 22.48 | 11.21 | 2.0 | CD27- | 678655 | NR_015382 | Hs.655224 | ENSG00000215039 |
| | | | | | AS1 | | | | |
| <1e−07 | <1e−07 | 154.15 | 39.97 | 3.86 | AP000547.3 | | | | |
| <1e−07 | <1e−07 | 24.89 | 8.65 | 2.88 | RNF208 | 727800 | NM_031297 | Hs.512767 | ENSG00000212864 |
| <1e−07 | <1e−07 | 40.8 | 12.31 | 3.31 | HEMGN | 55363 | NM_018437 | Hs.176626 | ENSG00000136929 |
| <1e−07 | <1e−07 | 17.12 | 6.74 | 2.54 | LANCL3 | 347404 | NM_001170331 | Hs.521932 | ENSG00000147036 |
| <1e−07 | <1e−07 | 101.81 | 32.01 | 3.18 | AP003068.2 | | | | |
| <1e−07 | <1e−07 | 39.34 | 15.82 | 2.4 | MAST4 | 375449 | NM_001164664 | Hs.595458 | ENSG00000069020 |
| <1e−07 | <1e−07 | 13.09 | 5.69 | 2.3 | EFHC2 | 80258 | NM_025184 | Hs.521953 | ENSG00000183690 |
| <1e−07 | <1e−07 | 226.84 | 109.49 | 2.07 | PPDPF | 79144 | NM_001353423 | Hs.79625 | ENSG00000125534 |
| <1e−07 | <1e−07 | 33.48 | 10.21 | 3.28 | RBPMS2 | 348093 | NM_194272 | Hs.436518 | ENSG00000166831 |
| <1e−07 | <1e−07 | 14.87 | 6.52 | 2.28 | MSRB3 | 253827 | NM_001031679 | Hs.339024 | ENSG00000174099 |
| <1e−07 | <1e−07 | 91.31 | 41.56 | 2.2 | ABCC3 | 8714 | NM_001144070 | Hs.463421 | ENSG00000108846 |
| <1e−07 | <1e−07 | 21.17 | 8.47 | 2.5 | SLFN14 | 342618 | NM_001129820 | Hs.591193 | ENSG00000236320 |
| <1e−07 | <1e−07 | 14.2 | 6.17 | 2.3 | ACER2 | 340485 | NM_001010887 | Hs.41379 | ENSG00000177076 |
| <1e−07 | <1e−07 | 13.22 | 6.17 | 2.14 | TMEM64 | 169200 | NM_001008495 | Hs.567759 | ENSG00000180694 |
| <1e−07 | <1e−07 | 39.68 | 14.29 | 2.78 | TSPAN9 | 10867 | NM_001168320 | Hs.504517 | ENSG00000011105 |
| <1e−07 | <1e−07 | 25.73 | 8.37 | 3.07 | SEC14L5 | 9717 | NM_014692 | Hs.512856 | ENSG00000103184 |
| <1e−07 | <1e−07 | 35.79 | 10.71 | 3.34 | GMPR | 2766 | NM_006877 | Hs.484741 | ENSG00000137198 |
| <1e−07 | <1e−07 | 12.98 | 5.84 | 2.22 | AC107223.1 | | | | |
| <1e−07 | <1e−07 | 52.03 | 25.83 | 2.01 | VDAC3 | 7419 | NM_001135694 | Hs.699301 | ENSG00000078668 |
| <1e−07 | <1e−07 | 19.66 | 7.24 | 2.72 | CTSW | 1521 | NM_001335 | Hs.416848 | ENSG00000172543 |
| <1e−07 | <1e−07 | 13.33 | 6.09 | 2.19 | CDKL1 | 8814 | NM_001282236 | Hs.280881 | ENSG00000100490 |
| <1e−07 | <1e−07 | 18.44 | 7.26 | 2.54 | CABP5 | 56344 | NM_016367 | Hs.117694 | ENSG00000105507 |
| <1e−07 | <1e−07 | 116.24 | 39.61 | 2.93 | CD9 | 928 | NM_001330312 | Hs.114286 | ENSG00000010278 |
| <1e−07 | <1e−07 | 73.42 | 28.59 | 2.57 | TMEM140 | 55281 | NM_018295 | Hs.521213 | ENSG00000146859 |
| <1e−07 | <1e−07 | 16.11 | 6.75 | 2.39 | PLOD2 | 5352 | NM_000935 | Hs.477866 | ENSG00000152952 |
| <1e−07 | <1e−07 | 25.31 | 11.73 | 2.16 | APLF | 200558 | NM_173545 | Hs.720369 | ENSG00000169621 |
| <1e−07 | <1e−07 | 27.6 | 13.21 | 2.09 | ARHGAP10 | 79658 | NM_024605 | Hs.368631 | ENSG00000071205 |
| <1e−07 | <1e−07 | 18.75 | 9.25 | 2.03 | TRAPPC2 | 6399 | NM_001011658 | Hs.592238 | ENSG00000196459 |
| <1e−07 | <1e−07 | 17.87 | 8.29 | 2.15 | LINC02284 | 1.03E+08 | XR 001750778 | | |
| <1e−07 | <1e−07 | 18.28 | 7.31 | 2.5 | AVPR1A | 552 | NM_000706 | Hs.2131 | ENSG00000166148 |
| <1e−07 | <1e−07 | 12.83 | 5.75 | 2.23 | SMIM5 | 643008 | NM_001162995 | Hs.528605 | ENSG00000204323 |
| <1e−07 | <1e−07 | 14.88 | 6.43 | 2.31 | RTN2 | 6253 | NM_005619 | Hs.47517 | ENSG00000125744 |
| <1e−07 | <1e−07 | 38.87 | 18.48 | 2.1 | UBL4A | 8266 | NM_014235 | Hs.76480 | ENSG00000102178 |
| <1e−07 | <1e−07 | 293.3 | 114.98 | 2.55 | RSU1 | 6251 | NM_012425 | Hs.524161 | ENSG00000148484 |
| <1e−07 | <1e−07 | 16.54 | 8.11 | 2.04 | OPHN1 | 4983 | NM_002547 | Hs.128824 | ENSG00000079482 |
| <1e−07 | <1e−07 | 94.03 | 35.07 | 2.68 | MGLL | 11343 | NM_001003794 | Hs.277035 | ENSG00000074416 |
| <1e−07 | <1e−07 | 21.02 | 8.05 | 2.61 | HOMER2 | 9455 | NM_004839 | Hs.578443 | ENSG00000103942 |
| <1e−07 | <1e−07 | 49.85 | 15.52 | 3.21 | PROS1 | 5627 | NM_000313 | Hs.64016 | ENSG00000184500 |
| <1e−07 | <1e−07 | 102.37 | 37.88 | 2.7 | MAP1A | 4130 | NM_002373 | Hs.194301 | ENSG00000166963 |
| <1e−07 | <1e−07 | 19.55 | 9.17 | 2.13 | KIFAP3 | 22920 | NM_001204514 | Hs.433442 | ENSG00000075945 |
| <1e−07 | <1e−07 | 15.59 | 6.23 | 2.5 | AL133444.1 | | | | |
| <1e−07 | <1e−07 | 51.72 | 24.43 | 2.12 | CDK2AP1 | 8099 | NM_001270433 | Hs.433201 | ENSG00000111328 |
| <1e−07 | <1e−07 | 13.52 | 5.86 | 2.31 | MFSD2B | 388931 | NM_001080473 | Hs.407482 | ENSG00000205639 |
| <1e−07 | <1e−07 | 11.68 | 5.41 | 2.16 | AL355073.1 | | | | |
| <1e−07 | <1e−07 | 15.48 | 7.47 | 2.07 | AGBL5 | 60509 | NM_001035507 | Hs.138207 | ENSG00000084693 |
| <1e−07 | <1e−07 | 24.36 | 9.36 | 2.6 | H2BC8 | 8339 | | | |
| <1e−07 | <1e−07 | 22.09 | 9.21 | 2.4 | LEPR | 3953 | NM_001003679 | Hs.23581 | ENSG00000116678 |
| <1e−07 | <1e−07 | 19.46 | 9.47 | 2.06 | ST7 | 7982 | NM_018412 | Hs.368131 | ENSG00000004866 |
| <1e−07 | <1e−07 | 64.96 | 23.36 | 2.78 | TSPAN18 | 90139 | NM_001031730 | Hs.385634 | ENSG00000157570 |
| <1e−07 | <1e−07 | 98.16 | 43.27 | 2.27 | MARCHF2 | 51257 | | | |
| <1e−07 | <1e−07 | 12.18 | 6.07 | 2.01 | LRP12 | 29967 | NM_001135703 | Hs.600630 | ENSG00000147650 |
| <1e−07 | <1e−07 | 39.39 | 19.52 | 2.02 | CDIP1 | 29965 | NM_001199054 | Hs.654653 | ENSG00000089486 |
| <1e−07 | <1e−07 | 36.09 | 12.93 | 2.79 | SYNM | 23336 | NM_015286 | Hs.207106 | ENSG00000182253 |
| <1e−07 | <1e−07 | 13.72 | 6.3 | 2.18 | TMEM185A | 84548 | NM_001174092 | Hs.522172 | ENSG00000269556 |
| <1e−07 | <1e−07 | 48.35 | 16.6 | 2.91 | ABLIM1 | 3983 | NM_001003407 | Hs.438236 | ENSG00000099204 |
| <1e−07 | <1e−07 | 21.46 | 8.11 | 2.65 | SLC24A3 | 57419 | NM_020689 | Hs.654790 | ENSG00000185052 |
| <1e−07 | <1e−07 | 12.21 | 5.31 | 2.3 | ADRA2A | 150 | NM_000681 | Hs.249159 | ENSG00000150594 |
| <1e−07 | <1e−07 | 16.8 | 8 | 2.1 | GCLM | 2730 | NM_001308253 | Hs.315562 | ENSG00000023909 |
| <1e−07 | <1e−07 | 120.17 | 53.5 | 2.25 | TSPAN33 | 340348 | NM_178562 | Hs.27267 | ENSG00000158457 |
| <1e−07 | <1e−07 | 160.46 | 70.39 | 2.28 | SLC40A1 | 30061 | NM_014585 | Hs.643005 | ENSG00000138449 |
| <1e−07 | <1e−07 | 161.36 | 78.74 | 2.05 | DAPP1 | 27071 | NM_001306151 | Hs.436271 | ENSG00000070190 |
| <1e−07 | <1e−07 | 10.97 | 5.07 | 2.16 | CFAP161 | 161502 | NM_001353365 | Hs.130979 | ENSG00000156206 |
| <1e−07 | <1e−07 | 21.94 | 7.78 | 2.82 | TGFB1I1 | 7041 | NM_001042454 | Hs.513530 | ENSG00000140682 |
| <1e−07 | <1e−07 | 45.17 | 20 | 2.26 | H2AC8 | 3012 | | | |
| <1e−07 | <1e−07 | 46.66 | 18.48 | 2.53 | ANKRD33B | 651746 | NM_001164440 | Hs.26039 | ENSG00000164236 |
| <1e−07 | <1e−07 | 89.38 | 42.69 | 2.09 | STMP1 | 647087 | NM_001130929 | Hs.200022 | ENSG00000243317 |
| <1e−07 | <1e−07 | 54.01 | 25.62 | 2.11 | FRMD3 | 257019 | NM_001244959 | Hs.127535 | ENSG00000172159 |
| <1e−07 | <1e−07 | 14.1 | 5.78 | 2.44 | NFIB | 4781 | NM_001190737 | Hs.644095 | ENSG00000147862 |
| <1e−07 | <1e−07 | 22.31 | 9.58 | 2.33 | TPTEP1 | 387590 | NR_001591 | Hs.474116 | ENSG00000100181 |
| <1e−07 | <1e−07 | 21.91 | 8.52 | 2.5' | SYTL4 | 94121 | NM_001129896 | Hs.592224 | ENSG00000102362 |
| <1e−07 | <1e−07 | 23.48 | 8.61 | 2.73 | SPOCD1 | 90853 | NM_001281987 | Hs.62604 | ENSG00000134668 |
| <1e−07 | <1e−07 | 354.94 | 128.92 | 2.75 | MAP3K7CL | 56911 | NM_001286617 | Hs.222802 | ENSG00000156265 |
| <1e−07 | <1e−07 | 13.93 | 6.19 | 2.25 | COL24A1 | 255631 | NM_001349955 | Hs.659516 | ENSG00000171502 |
| <1e−07 | <1e−07 | 26.49 | 12.08 | 2.19 | KIF3C | 3797 | NM_002254 | Hs.21611 | ENSG00000084731 |
| <1e−07 | <1e−07 | 35.36 | 13.52 | 2.62 | TBXA2R | 6915 | NM_001060 | Hs.442530 | ENSG00000006638 |
| <1e−07 | <1e−07 | 28.78 | 11.59 | 2.48 | AIG1 | 51390 | NM_001286587 | Hs.567501 | ENSG00000146416 |
| <1e−07 | <1e−07 | 24.6 | 8.63 | 2.85 | PRKAR1B | 5575 | NM_001164758 | Hs.520851 | ENSG00000188191 |
| <1e−07 | <1e−07 | 13.81 | 6.57 | 2.1 | MYOM1 | 8736 | NM_003803 | Hs.464469 | ENSG00000101605 |
| <1e−07 | <1e−07 | 224.29 | 63.9 | 3.51 | AP001189.1 | | | | |
| <1e−07 | <1e−07 | 12.52 | 5.87 | 2.13 | AFAP1L2 | 84632 | NM_001001936 | Hs.501106 | ENSG00000169129 |
| <1e−07 | <1e−07 | 12.64 | 5.92 | 2.13 | SLC6A4 | 6532 | NM_001045 | Hs.29792 | ENSG00000108576 |
| <1e−07 | <1e−07 | 281.53 | 136.25 | 2.07 | RUFY1 | 80230 | NM_001040451 | Hs.306769 | ENSG00000176783 |
| <1e−07 | <1e−07 | 92.71 | 29 | 3.2 | GP9 | 2815 | NM_000174 | Hs.1144 | ENSG00000169704 |
| <1e−07 | <1e−07 | 17.03 | 7.34 | 2.32 | PSD3 | 23362 | NM_015310 | Hs.434255 | ENSG00000156011 |
| <1e−07 | <1e−07 | 11.39 | 5.62 | 2.03 | MISP3 | 113230 | NM_001291291 | Hs.372775 | ENSG00000141854 |
| <1e−07 | <1e−07 | 119.71 | 57.74 | 2.07 | YWHAH | 7533 | NM_003405 | Hs.226755 | ENSG00000128245 |
| <1e−07 | <1e−07 | 21.37 | 7.41 | 2.88 | TMEM158 | 25907 | NM_015444 | Hs.740403 | ENSG00000249992 |
| <1e−07 | <1e−07 | 13.05 | 5.93 | 2.2 | INPP4B | 8821 | NM_001101669 | Hs.176376 | ENSG00000109452 |
| <1e−07 | <1e−07 | 62 | 26.09 | 2.38 | PNMA1 | 9240 | NM_006029 | Hs.194709 | ENSG00000176903 |
| <1e−07 | <1e−07 | 21.62 | 8.67 | 2.49 | ZNF542P | 147947 | NM_194319 | Hs.467326 | ENSG00000240225 |
| <1e−07 | <1e−07 | 1596.27 | 676.93 | 2.36 | MT- | 4541 | | | |
| | | | | | ND6 | | | | |
| <1e−07 | <1e−07 | 224.85 | 87.13 | 2.58 | MT- | 4571 | | | |
| | | | | | TP | | | | |
| <1e−07 | <1e−07 | 32.37 | 12.74 | 2.54 | PTPRS | 5802 | NM_002850 | Hs.744928 | ENSG00000105426 |
| <1e−07 | <1e−07 | 18.16 | 7.1 | 2.5 | AQP10 | 89872 | NM_080429 | Hs.259048 | ENSG00000143595 |
| <1e−07 | <1e−07 | 11.27 | 5.49 | 2.05 | MCPH | 1.01E+08 | NR_125386 | Hs.490892 | |
| | | | | | 1-AS1 | | | | |
| <1e−07 | <1e−07 | 36.97 | 12.24 | 3.02 | SAMD14 | 201191 | NM_001257359 | Hs.567769 | ENSG00000167100 |
| <1e−07 | <1e−07 | 62.73 | 31.33 | 2 | IGF2BP2 | 10644 | NM_001007225 | Hs.35354 | ENSG00000073792 |
| <1e−07 | <1e−07 | 128.73 | 53.12 | 2.42 | NEXN | 91624 | NM_001172309 | Hs.612385 | ENSG00000162614 |
| <1e−07 | <1e−07 | 18.45 | 7.9 | 2.33 | RAB30 | 27314 | NM_001286059 | Hs.40758 | ENSG00000137502 |
| <1e−07 | <1e−07 | 22.02 | 8.41 | 2.62 | CLDN5 | 7122 | NM_001130861 | Hs.505337 | ENSG00000184113 |
| <1e−07 | 2.94E−06 | 19.49 | 9.03 | 2.16 | FNBP1L | 54874 | NM_001024948 | Hs.134060 | ENSG00000137942 |
| <1e−07 | 2.94E−06 | 35.66 | 14.6 | 2.44 | MT-TI | 4565 | | | |
| <1e−07 | 2.94E−06 | 24.32 | 10.51 | 2.31 | VWF | 7450 | NM_000552 | Hs.440848 | ENSG00000110799 |
| <1e−07 | 2.94E−06 | 426.41 | 164.18 | 2.6 | ITGA2B | 3674 | NM_000419 | Hs.411312 | ENSG00000005961 |
| <1e−07 | 2.94E−06 | 13.8 | 6.09 | 2.26 | ITGA6 | 3655 | NM_000210 | Hs.133397 | ENSG00000091409 |
| <1e−07 | 2.94E−06 | 13.31 | 6.26 | 2.13 | HGD | 3081 | NM_000187 | Hs.368254 | ENSG00000113924 |
| 1.00E−07 | 2.94E−06 | 12.07 | 5.79 | 2.08 | ITGA2 | 3673 | NM_002203 | Hs.482077 | ENSG00000164171 |
| 1.00E−07 | 2.94E−06 | 14.33 | 6.66 | 2.15 | SERPINE1 | 5054 | NM_000602 | Hs.414795 | ENSG00000106366 |
| 1.00E−07 | 2.94E−06 | 47.52 | 18.7 | 2.54 | MT- | 4574 | | | |
| | | | | | TS1 | | | | |
| 1.00E−07 | 2.94E−06 | 102.21 | 24.68 | 4.14 | PF4V1 | 5197 | NM_002620 | Hs.72933 | ENSG00000109272 |
| 2.00E−07 | 5.64E−06 | 24.15 | 11.64 | 2.07 | ACVR2A | 92 | NM_001278579 | Hs.470174 | ENSG00000121989 |
| 2.00E−07 | 5.64E−06 | 11.14 | 5.45 | 2.04 | F2RL3 | 9002 | NM_003950 | Hs.137574 | ENSG00000127533 |
| 2.00E−07 | 5.64E−06 | 15.21 | 6.91 | 2.2 | MYCT1 | 80177 | NM_025107 | Hs.18160 | ENSG00000120279 |
| 2.00E−07 | 5.64E−06 | 29.31 | 9.56 | 3.07 | PDZK1IP1 | 10158 | NM_005764 | Hs.431099 | ENSG00000162366 |
| 2.00E−07 | 5.64E−06 | 11.41 | 5.67 | 2.0 | MLC1 | 23209 | NM_015166 | Hs.517729 | ENSG00000100427 |
| 2.00E−07 | 5.64E−06 | 13.33 | 6.65 | 2 | CRACD | 57482 | | | |
| 2.00E−07 | 5.64E−06 | 16.37 | 7.54 | 2.17 | AC127502.2 | | | | |
| 2.00E−07 | 5.64E−06 | 11.93 | 5.64 | 2.12 | GRHL1 | 29841 | NM_198182 | Hs.418493 | ENSG00000134317 |
| 3.00E−07 | 8.16E−06 | 16.43 | 6.97 | 2.36 | GP5 | 2814 | NM_004488 | Hs.73734 | ENSG00000178732 |
| 3.00E−07 | 8.16E−06 | 26.29 | 9.43 | 2.79 | AC123912.4 | | | | |
| 3.00E−07 | 8.16E−06 | 35.96 | 15.79 | 2.28 | GNB5 | 10681 | NM_006578 | Hs.155090 | ENSG00000069966 |
| 4.00E−07 | 1.05E−05 | 30.88 | 8.68 | 3.56 | OSBP2 | 23762 | NM_001003812 | Hs.517546 | ENSG00000184792 |
| 4.00E−07 | 1.05E−05 | 13.29 | 6.36 | 2.09 | PRKG1 | 5592 | NM_001098512 | Hs.407535 | ENSG00000185532 |
| 5.00E−07 | 1.28E−05 | 23.72 | 9.99 | 2.37 | IRS1 | 3667 | NM_005544 | Hs.471508 | ENSG00000169047 |
| 8.00E−07 | 1.95E−05 | 34.05 | 8.95 | 3.8 | RPL23AP7 | 118433 | NR_000029 | Hs.595056 | |
| 1.00E−06 | 2.39E−05 | 42.52 | 19.81 | 2.15 | AFAP1 | 60312 | NM_001134647 | Hs.529369 | ENSG00000196526 |
| 1.20E−06 | 2.81E−05 | 13.25 | 5.98 | 2.22 | POLR3G | 10622 | NM_006467 | Hs.282387 | ENSG00000113356 |
| 1.60E−06 | 3.61E−05 | 28.06 | 10.71 | 2.62 | FSTL1 | 11167 | NM_007085 | Hs.269512 | ENSG00000163430 |
| 1.80E−06 | 4.03E−05 | 18.58 | 8.73 | 2.13 | ANKRD9 | 122416 | NM_001348651 | Hs.432945 | ENSG00000156381 |
| 2.60E−06 | 5.65E−06 | 93.38 | 37.52 | 2.49 | MT- | 4553 | | | |
| | | | | | TA | | | | |
| 2.80E−06 | 6.05E−05 | 24.06 | 11.42 | 2.11 | DBN1 | 1627 | NM_004395 | Hs.130316 | ENSG00000113758 |
| 3.30E−06 | 7.01E−05 | 19.54 | 9.69 | 2.02 | P2RY1 | 5028 | NM_002563 | Hs.79881 | ENSG00000169860 |
| 4.40E−06 | 9.00E−05 | 22.72 | 9.58 | 2.37 | MT- | 4572 | | | |
| | | | | | TQ | | | | |
| 4.60E−06 | 9.40E−05 | 77.34 | 28.64 | 2.7 | TENT5C | 54855 | | | |
| 4.70E−06 | 9.55E−05 | 96.56 | 41.82 | 2.31 | TNS1 | 7145 | NM_001308022 | Hs.471381 | ENSG00000079308 |
| 4.80E−06 | 9.70E−05 | 21.98 | 10.15 | 2.17 | KIFC3 | 3801 | NM_001130099 | Hs.23131 | ENSG00000140859 |
| 4.80E−06 | 9.70E−05 | 41.48 | 16.46 | 2.52 | HSPB1 | 3315 | NM_001540 | Hs.520973 | ENSG00000106211 |
| 5.60E−06 | 0.000111 | 38.58 | 17.04 | 2.26 | GATA2 | 2624 | NM_001145661 | Hs.367725 | ENSG00000179348 |
| 1.74E−05 | 0.000301 | 109.89 | 53.4 | 2.06 | MT- | 4576 | | | |
| | | | | | TT | | | | |
| 2.17E−05 | 0.000362 | 13.27 | 6.59 | 2.01 | ANKH | 56172 | NM_054027 | Hs.156727 | ENSG00000154122 |
| 2.23E−05 | 0.000369 | 38.77 | 6.66 | 5.82 | HBA1 | 3039 | NM_000558 | Hs.449630 | ENSG00000206172 |
| 2.45E−05 | 4.00E−04 | 16.39 | 7.54 | 2.17 | MAP1B | 4131 | NM_001324255 | Hs.335079 | ENSG00000131711 |
| 5.76E−05 | 0.000837 | 36.05 | 105.25 | 0.34 | RNU4-1 | 26835 | NR_003925 | | ENSG00000200795 |
| 7.18E−05 | 0.00101 | 263.2 | 129.14 | 2.04 | MTND2P28 | 1.01E+08 | | | |
| 0.0001667 | 0.00204 | 24.78 | 11.77 | 2.11 | PALM2AKA | P2445815 | | | |
| 0.0003038 | 0.00333 | 13.1 | 6.54 | 2 | MAGI2- | 1.01E+08 | NR_038343 | Hs.31474 | ENSG00000234456 |
| | | | | | AS3 | | | | |
| 0.0007045 | 0.000667 | 40.73 | 15.17 | 2.69 | DDX11L10 | 1E+08 | NR_045117 | Hs.592089 | ENSG00000233614 |
| 0.0008146 | 0.00751 | 46.08 | 8.57 | 5.38 | HBB | 3043 | NM_000518 | Hs.523443 | ENSG00000244734 |
| 0.0009432 | 0.00848 | 19.13 | 9.47 | 2.02 | TNF | 7124 | NM_000594 | Hs.241570 | ENSG00000232810 |

TABLE 2
Differentially Expressed Genes (DEG) that contain a fold change ≥ 1.5

| | | | Geom | | | | | | |
|---|---|---|---|---|---|---|---|---|---|
| | | Geom | mean | | | | | | |
| | | mean | of | | | | | | |
| | | of | intensities | | | | | | |
| Parametric | | intensities | PAF- | Fold- | | Entrez | | | |
| p-value | FDR | Cdmono | Cdmono | change | UniqueID | ID | Accession | UGCluster | Ensembl |
| <1e−07 | <1e−07 | 225.46 | 74.9 | 3.01 | ODC1 | 4953 | NM_001287188 | Hs.467701 | ENSG00000115758 |
| <1e−07 | <1e−07 | 234.01 | 77.19 | 3.03 | MTURN | 222166 | NM_152793 | Hs.200100 | ENSG00000180354 |
| <1e−07 | <1e−07 | 139.21 | 53.68 | 2.59 | PCGF5 | 84333 | NM_001256549 | Hs.500512 | ENSG00000180628 |
| <1e−07 | <1e−07 | 648.43 | 300.89 | 2.16 | ITGB1 | 3688 | NM_002211 | Hs.643813 | ENSG00000150093 |
| <1e−07 | <1e−07 | 69.6 | 20.5 | 3.39 | PBX1 | 5087 | NM_001204961 | Hs.557097 | ENSG00000185630 |
| <1e−07 | <1e−07 | 922.35 | 492.89 | 1.87 | GNAS | 2778 | NM_000516 | Hs.125898 | ENSG00000087460 |
| <1e−07 | <1e−07 | 456.51 | 152.27 | 3 | H2AC6 | | | | ENSG00000180573 |
| <1e−07 | <1e−07 | 288.87 | 78.28 | 3.69 | PDLIMI | 9124 | NM_020992 | Hs.368525 | ENSG00000107438 |
| <1e−07 | <1e−07 | 436.15 | 134.19 | 3.25 | GRAP2 | 9402 | NM_001291824 | Hs.517499 | ENSG00000100351 |
| <1e−07 | <1e−07 | 260.27 | 125.55 | 2.07 | TLK1 | 9874 | NM_001136554 | Hs.744917 | ENSG00000198586 |
| <1e−07 | <1e−07 | 194.9 | 65.35 | 2.98 | RGS10 | 6001 | NM_001005339 | Hs.501200 | ENSG00000148908 |
| <1e−07 | <1e−07 | 402.3 | 108.12 | 3.72 | SH3BGRL2 | 83699 | NM_031469 | Hs.302772 | ENSG00000198478 |
| <1e−07 | <1e−07 | 501.03 | 174.85 | 2.87 | KIF2A | 3796 | NM_001098511 | Hs.558351 | ENSG00000068796 |
| <1e−07 | <1e−07 | 290.41 | 106.23 | 2.73 | RNF11 | 26994 | NM_014372 | Hs.309641 | ENSG00000123091 |
| <1e−07 | <1e−07 | 493.87 | 185.42 | 2.66 | MAX | 4149 | NM_001271068 | Hs.285354 | ENSG00000125952 |
| <1e−07 | <1e−07 | 25.55 | 7.06 | 3.62 | VEPH1 | 79674 | NM_001167911 | Hs.658046 | ENSG00000197415 |
| <1e−07 | <1e−07 | 51.48 | 16.62 | 3.1 | TTC7B | 145567 | NM_001010854 | Hs.655697 | ENSG00000165914 |
| <1e−07 | <1e−07 | 657.3 | 322.21 | 2.04 | NAPIL1 | 4673 | NM_001307924 | Hs.524599 | ENSG00000187109 |
| <1e−07 | <1e−07 | 198.24 | 90.74 | 2.18 | CMPK1 | 51727 | NM_001136140 | Hs.731647 | ENSG00000162368 |
| <1e−07 | <1e−07 | 102.48 | 30.16 | 3.4 | LGALSL | 29094 | NM_014181 | Hs.372208 | ENSG00000119862 |
| <1e−07 | <1e−07 | 55.18 | 16.9 | 3.27 | MEIS1 | 4211 | NM_002398 | Hs.526754 | ENSG00000143995 |
| <1e−07 | <1e−07 | 191.13 | 53.52 | 3.57 | LTBP1 | 4052 | NM_000627 | Hs.619315 | ENSG00000049323 |
| <1e−07 | <1e−07 | 258.72 | 76.2 | 3.4 | CTTN | 2017 | NM_001184740 | Hs.596164 | ENSG00000085733 |
| <1e−07 | <1e−07 | 56.27 | 15.52 | 3.63 | CDC14B | 8555 | NM_001077181 | Hs.40582 | ENSG00000081377 |
| <1e−07 | <1e−07 | 1511.38 | 381.79 | 3.96 | CCL5 | 6352 | NM_001278736 | Hs.514821 | ENSG00000271503 |
| <1e−07 | <1e−07 | 52.46 | 17.79 | 2.95 | TSPOAP1AS1 | 100506779 | NR_038410 | Hs.718477 | ENSG00000265148 |
| <1e−07 | <1e−07 | 504.84 | 173.22 | 2.91 | PTGS1 | 5742 | NM_000962 | Hs.201978 | ENSG00000095303 |
| <1e−07 | <1e−07 | 114.93 | 40.11 | 2.8 | CCDC92 | 80212 | NM_001304957 | Hs.114111 | ENSG00000119242 |
| <1e−07 | <1e−07 | 26.78 | 7.02 | 3.81 | TRAPPC3L | 100128327 | NM_001139444 | Hs.134795 | ENSG00000173626 |
| <1e−07 | <1e−07 | 28.3 | 8.64 | 3.28 | RGS6 | 9628 | NM_001204416 | Hs.509872 | ENSG00000182732 |
| <1e−07 | <1e−07 | 108.52 | 31.65 | 3.43 | CTDSPL | 10217 | NM_001008392 | Hs.475963 | ENSG00000144677 |
| <1e−07 | <1e−07 | 127.5 | 37.33 | 3.42 | GFI1B | 8328 | NM_001135031 | Hs.553160 | ENSG00000165702 |
| <1e−07 | <1e−07 | 26.77 | 8.32 | 3.22 | ATP9A | 10079 | NM_006045 | Hs.649234 | ENSG00000054793 |
| <1e−07 | <1e−07 | 139.72 | 38.28 | 3.65 | ENDOD1 | 23052 | NM_015036 | Hs.167115 | ENSG00000149218 |
| <1e−07 | <1e−07 | 104.95 | 28.87 | 3.64 | SELP | 6403 | NM_003005 | Hs.73800 | ENSG00000174175 |
| <1e−07 | <1e−07 | 711.36 | 233.37 | 3.05 | OST4 | 100128731 | NM_001134693 | Hs.502948 | ENSG00000228474 |
| <1e−07 | <1e−07 | 634.26 | 212.68 | 2.98 | PRKAR2B | 5577 | NM_002736 | Hs.433068 | ENSG00000005249 |
| <1e−07 | <1e−07 | 198.26 | 86.66 | 2.29 | MOB1B | 92597 | NM_001244766 | Hs.691454 | ENSG00000173542 |
| <1e−07 | <1e−07 | 312.59 | 152.53 | 2.05 | MPP1 | 4354 | NM_001166460 | Hs.496984 | ENSG00000130830 |
| <1e−07 | <1e−07 | 181.22 | 53.45 | 3.39 | FAXDC2 | 10826 | NM_016348 | Hs.519694 | ENSG00000170271 |
| <1e−07 | <1e−07 | 34.87 | 10.35 | 3.37 | PARD3 | 56288 | NM_001184785 | Hs.131489 | ENSG00000148498 |
| <1e−07 | <1e−07 | 68.31 | 27.4 | 2.49 | SEPTIN11 | | | | ENSG00000138758 |
| <1e−07 | <1e−07 | 41.14 | 12.1 | 3.4 | EGF | 1950 | NM_001178130 | Hs.419815 | ENSG00000138798 |
| <1e−07 | <1e−07 | 309.01 | 88.44 | 3.49 | C2orf88 | 84281 | NM_001042519 | Hs.389311 | ENSG00000187699 |
| <1e−07 | <1e−07 | 181.68 | 52.06 | 3.49 | PDE5A | 8654 | NM_001083 | Hs.647971 | ENSG0000018735 |
| <1e−07 | <1e−07 | 739.62 | 223.3 | 3.31 | SPARC | 6678 | NM_001309443 | Hs.111779 | ENSG00000113140 |
| <1e−07 | <1e−07 | 280.8 | 166.19 | 1.69 | HMGB1 | 3146 | NM_001313892 | Hs.434102 | ENSG00000189403 |
| <1e−07 | <1e−07 | 250.44 | 80.62 | 3.11 | CD226 | 10666 | NM_001303618 | Hs.660130 | ENSG00000150637 |
| <1e−07 | <1e−07 | 997.18 | 328.35 | 3.04 | CAVIN2 | 8436 | NM_004657 | Hs.26530 | ENSG00000168497 |
| <1e−07 | <1e−07 | 180.53 | 55.65 | 3.24 | MMD | 23531 | NM_012329 | Hs.463483 | ENSG00000108960 |
| <1e−07 | <1e−07 | 524.63 | 204.32 | 2.57 | LIMS1 | 3987 | NM_001193482 | Hs.597715 | ENSG00000169756 |
| <1e−07 | <1e−07 | 196.67 | 53.7 | 3.66 | TREML1 | 340205 | NM_001271807 | Hs.117331 | ENSG00000161911 |
| <1e−07 | <1e−07 | 118.11 | 36.29 | 3.25 | ACRBP | 84519 | NM_032489 | Hs.123239 | ENSG00000111644 |
| <1e−07 | <1e−07 | 61.01 | 18.48 | 3.3 | ARHGAP6 | 395 | NM_001174 | Hs.435291 | ENSG00000047648 |
| <1e−07 | <1e−07 | 231.2 | 67.3 | 3.44 | GNG11 | 2791 | NM_004126 | Hs.83381 | ENSG00000127920 |
| <1e−07 | <1e−07 | 151.35 | 47.78 | 3.17 | DNM3 | 26052 | NM_001136127 | Hs.654775 | ENSG00000197959 |
| <1e−07 | <1e−07 | 169.32 | 53.07 | 3.19 | MYLK | 4638 | NM_001321309 | Hs.477375 | ENSG00000065534 |
| <1e−07 | <1e−07 | 218.55 | 78.29 | 2.79 | PGRMC1 | 10857 | NM_001282621 | Hs.90061 | ENSG00000101856 |
| <1e−07 | <1e−07 | 27.15 | 7.75 | 3.5 | VIL1 | 7429 | NM_007127 | Hs.654595 | ENSG00000127831 |
| <1e−07 | <1e−07 | 49.36 | 14.18 | 3.48 | TC2N | 123036 | NM_001128595 | Hs.510262 | ENSG00000165929 |
| <1e−07 | <1e−07 | 67.34 | 28.3 | 2.38 | PTK2 | 5747 | NM_001199649 | Hs.395482 | ENSG00000169398 |
| <1e−07 | <1e−07 | 793.47 | 233.6 | 3.4 | MPIG6B | 80739 | NM_025260 | Hs.247879 | ENSG00000204420 |
| <1e−07 | <1e−07 | 144.48 | 60.52 | 2.39 | PRDX6 | 9588 | NM_004905 | Hs.120 | ENSG00000117592 |
| <1e−07 | <1e−07 | 144.37 | 52.29 | 2.76 | DAB2 | 1601 | NM_001244871 | Hs.696631 | ENSG00000153071 |
| <1e−07 | <1e−07 | 55.51 | 16.25 | 3.42 | PLA2G12A | 81579 | NM_030821 | Hs.389452 | ENSG00000123739 |
| <1e−07 | <1e−07 | 52.44 | 16.06 | 3.27 | H2BC11 | 8970 | | | |
| <1e−07 | <1e−07 | 170.95 | 81.01 | 2.11 | LRBA | 987 | NM_001199282 | Hs.480938 | ENSG00000198589 |
| <1e−07 | <1e−07 | 112.29 | 36.03 | 3.12 | TAL1 | 6886 | NM_001287347 | Hs.705618 | ENSG00000162367 |
| <1e−07 | <1e−07 | 156.46 | 50.92 | 3.07 | ARHGEF12 | 23365 | NM_001198665 | Hs.24598 | ENSG00000196914 |
| <1e−07 | <1e−07 | 480.2 | 164.97 | 2.91 | RGS18 | 64407 | NM_130782 | Hs.440890 | ENSG00000150681 |
| <1e−07 | <1e−07 | 202 | 54.54 | 3.7 | MFAP3L | 9848 | NM_001009554 | Hs.593942 | ENSG00000198948 |
| <1e−07 | <1e−07 | 302.62 | 93.28 | 3.24 | CXCR2P1 | 3580 | NR_002712 | Hs.647858 | |
| <1e−07 | <1e−07 | 88.94 | 28.4 | 3.13 | ELOVL7 | 79993 | NM_001104558 | Hs.274256 | ENSG00000164181 |
| <1e−07 | <1e−07 | 2357.19 | 783.27 | 3.0 | TUBB1 | 81027 | NM_030773 | Hs.303023 | ENSG00000101162 |
| <1e−07 | <1e−07 | 146.31 | 61.18 | 2.39 | ANKRD28 | 23243 | NM_001195098 | Hs.335239 | ENSG00000206560 |
| <1e−07 | <1e−07 | 75.77 | 28.89 | 2.62 | PRUNE1 | 58497 | NM_001303229 | Hs.78524 | ENSG00000143363 |
| <1e−07 | <1e−07 | 59.1 | 16.88 | 3.5 | GUCY1A1 | 2982 | NM_000856 | Hs.24258 | ENSG00000164116 |
| <1e−07 | <1e−07 | 188.85 | 94.42 | 2 | MAPRE2 | 10982 | NM_001143826 | Hs.532824 | ENSG00000166974 |
| <1e−07 | <1e−07 | 71.59 | 21.76 | 3.29 | CPNE5 | 57699 | NM_001314017 | Hs.372129 | ENSG00000124772 |
| <1e−07 | <1e−07 | 203.6 | 70.56 | 2.89 | STON2 | 85439 | NM_001256430 | Hs.14248 | ENSG00000140022 |
| <1e−07 | <1e−07 | 71.21 | 21.53 | 3.31 | GUCY1B1 | 2983 | NM_000857 | Hs.77890 | ENSG00000061918 |
| <1e−07 | <1e−07 | 199.5 | 93.7 | 2.13 | ARHGAP21 | 57584 | NM_020824 | Hs.524195 | ENSG00000107863 |
| <1e−07 | <1e−07 | 176.94 | 78.6 | 2.25 | ZNF185 | 7739 | NM_001178106 | Hs.16622 | ENSG00000147394 |
| <1e−07 | <1e−07 | 101.9 | 29.08 | 3.5 | BEX3 | 27018 | NM_001282674 | Hs.448588 | ENSG00000166681 |
| <1e−07 | <1e−07 | 49.14 | 14.77 | 3.33 | SVIP | 258010 | NM_001320340 | Hs.349096 | ENSG00000198168 |
| <1e−07 | <1e−07 | 29.66 | 10.23 | 2.9 | MED12L | 116931 | NM_053002 | Hs.744234 | ENSG00000144893 |
| <1e−07 | <1e−07 | 260.94 | 108.07 | 2.41 | TUBA4A | 7277 | NM_001278552 | Hs.75318 | ENSG00000127824 |
| <1e−07 | <1e−07 | 103.06 | 28.78 | 3.58 | ABLIM3 | 22885 | NM_001301015 | Hs.49688 | ENSG00000173210 |
| <1e−07 | <1e−07 | 102.92 | 29.04 | 3.54 | BEND2 | 139105 | NM_001184767 | Hs.403802 | ENSG00000177324 |
| <1e−07 | <1e−07 | 48.38 | 14.44 | 3.35 | WHAMMP3 | 339005 | NR_003521 | Hs.212670 | |
| <1e−07 | <1e−07 | 75.13 | 22.06 | 3.41 | PEAR1 | 375033 | NM_001080471 | Hs.142003 | ENSG00000187800 |
| <1e−07 | <1e−07 | 696.69 | 341 | 2.04 | RAP1B | 5908 | NM_001010942 | Hs.369920 | ENSG00000127314 |
| <1e−07 | <1e−07 | 98.09 | 34.28 | 2.86 | ASAP2 | 8853 | NM_001135191 | Hs.555902 | ENSG00000151693 |
| <1e−07 | <1e−07 | 30.52 | 8.81 | 3.4 | RAB6B | 51560 | NM_016577 | Hs.715344 | ENSG00000154917 |
| <1e−07 | <1e−07 | 85.74 | 23.86 | 3.59 | PCSK6 | 5046 | NM_001291309 | Hs.498494 | ENSG00000140479 |
| <1e−07 | <1e−07 | 53.69 | 21.81 | 2.46 | MLH3 | 27030 | NM_001040108 | Hs.436650 | ENSG00000119684 |
| <1e−07 | <1e−07 | 43.3 | 13.04 | 3.32 | BANK1 | 55024 | NM_001083907 | Hs.480400 | ENSG00000153064 |
| <1e−07 | <1e−07 | 647.05 | 172 | 3.76 | PF4 | 5196 | NM_002619 | Hs.81564 | ENSG00000163737 |
| <1e−07 | <1e−07 | 247.62 | 95.51 | 2.59 | NCK2 | 8440 | NM_001004720 | Hs.529244 | ENSG00000071051 |
| <1e−07 | <1e−07 | 233.6 | 94.98 | 2.46 | TSC22D1 | 8848 | NM_001243797 | Hs.436383 | ENSG00000102804 |
| <1e−07 | <1e−07 | 15.32 | 5.9 | 2.6 | C12orf76 | 400073 | NM_207435 | Hs.44817 | ENSG00000174456 |
| <1e−07 | <1e−07 | 126.71 | 47.6 | 2.66 | ARHGAP18 | 93663 | NM_033515 | Hs.486458 | ENSG00000146376 |
| <1e−07 | <1e−07 | 1252.05 | 446.29 | 2.81 | F13A1 | 2162 | NM_000129 | Hs.335513 | ENSG00000124491 |
| <1e−07 | <1e−07 | 160.72 | 62.6 | 2.57 | STOM | 2040 | NM_001270526 | Hs.253903 | ENSG00000148175 |
| <1e−07 | <1e−07 | 80.83 | 22.89 | 3.53 | CA2 | 760 | NM_000067 | Hs.155097 | ENSG00000104267 |
| <1e−07 | <1e−07 | 136.34 | 55.53 | 2.46 | CNST | 163882 | NM_001139459 | Hs.368353 | ENSG00000162852 |
| <1e−07 | <1e−07 | 388.34 | 198.44 | 1.96 | ANO6 | 196527 | NM_001025356 | Hs.505339 | ENSG00000177119 |
| <1e−07 | <1e−07 | 910.88 | 397.49 | 2.29 | VCL | 7414 | NM_003373 | Hs.643896 | ENSG00000035403 |
| <1e−07 | <1e−07 | 99.12 | 29.88 | 3.32 | PKHD1L1 | 93035 | NM_177531 | Hs.170128 | ENSG00000205038 |
| <1e−07 | <1e−07 | 21.41 | 9.44 | 2.27 | AL365361.1 | | | | |
| <1e−07 | <1e−07 | 222.61 | 103.44 | 2.15 | RAB11A | 8766 | NM_001206836 | Hs.321541 | ENSG00000103769 |
| <1e−07 | <1e−07 | 223.36 | 105.04 | 2.13 | EIF2AK1 | 27102 | NM_001134335 | Hs.520205 | ENSG00000086232 |
| <1e−07 | <1e−07 | 96.44 | 32.29 | 2.99 | TPM1 | 7168 | NM_000366 | Hs.133892 | ENSG00000140416 |
| <1e−07 | <1e−07 | 23.8 | 7.77 | 3.06 | AC114752.2 | | | | |
| <1e−07 | <1e−07 | 202.96 | 63.17 | 3.21 | GNAZ | 2781 | NM_002073 | Hs.584760 | ENSG00000128266 |
| <1e−07 | <1e−07 | 97.4 | 51.33 | 1.9 | RYBP | 23429 | NM_012234 | Hs.7910 | ENSG00000163602 |
| <1e−07 | <1e−07 | 63.96 | 20.08 | 3.19 | IGF2BP3 | 10643 | NM_006547 | Hs.700696 | ENSG00000136231 |
| <1e−07 | <1e−07 | 69.34 | 20.97 | 3.31 | TMEM40 | 55287 | NM_001284406 | Hs.475502 | ENSG00000088726 |
| <1e−07 | <1e−07 | 257.25 | 72.38 | 3.55 | RAB27B | 5874 | NM_004163 | Hs.25318 | ENSG00000041353 |
| <1e−07 | <1e−07 | 599.89 | 154.93 | 3.87 | MYL9 | 10398 | NM_006097 | Hs.504687 | ENSG00000101335 |
| <1e−07 | <1e−07 | 340.8 | 105.49 | 3.23 | TRIM58 | 25893 | NM_015431 | Hs.269151 | ENSG00000162722 |
| <1e−07 | <1e−07 | 57.17 | 24.38 | 2.34 | PRKACB | 5567 | NM_001242857 | Hs.487325 | ENSG00000142875 |
| <1e−07 | <1e−07 | 27.88 | 9.32 | 2.99 | NCKAP1 | 10787 | NM_013436 | Hs.603732 | ENSG00000061676 |
| <1e−07 | <1e−07 | 33.29 | 10.41 | 3.2 | XK | 7504 | NM_021083 | Hs.78919 | ENSG00000047597 |
| <1e−07 | <1e−07 | 889.4 | 568.75 | 1.56 | YWHAZ | 7534 | NM_001135699 | Hs.492407 | ENSG00000164924 |
| <1e−07 | <1e−07 | 114.08 | 44.66 | 2.55 | NT5C3A | 51251 | NM_001002009 | Hs.487933 | ENSG00000122643 |
| <1e−07 | <1e−07 | 477.12 | 261.26 | 1.83 | MTPN | 136319 | NM_145808 | Hs.602015 | ENSG00000105887 |
| <1e−07 | <1e−07 | 33.29 | 9.36 | 3.55 | LCN2 | 3934 | NM_005564 | Hs.204238 | ENSG00000148346 |
| <1e−07 | <1e−07 | 45.21 | 14.08 | 3.21 | GP6 | 51206 | NM_001083899 | Hs.661752 | ENSG00000088053 |
| <1e−07 | <1e−07 | 50.43 | 15.69 | 3.21 | RHOBTB1 | 9886 | NM_001242359 | Hs.737374 | ENSG00000072422 |
| <1e−07 | <1e−07 | 42.51 | 17.4 | 2.44 | MCUR1 | 63933 | NM_001031713 | Hs.214043 | ENSG00000050393 |
| <1e−07 | <1e−07 | 32.17 | 13.05 | 2.47 | SIAE | 54414 | NM_001199922 | Hs.10056 | ENSG00000110013 |
| <1e−07 | <1e−07 | 40.64 | 13.35 | 3.04 | H2AC11 | 8969 | | | |
| <1e−07 | <1e−07 | 3432.04 | 1274.28 | 2.69 | NRGN | 4900 | NM_001126181 | Hs.524116 | ENSG00000154146 |
| <1e−07 | <1e−07 | 14 | 6.04 | 2.32 | ACTR3B | 57180 | NM_001040135 | Hs.647117 | ENSG00000133627 |
| <1e−07 | <1e−07 | 27.14 | 8.24 | 3.3 | SPX | 80763 | NM_030572 | Hs.130692 | ENSG00000134548 |
| <1e−07 | <1e−07 | 65.98 | 21.6 | 3.05 | SLA2 | 84174 | NM_032214 | Hs.713578 | ENSG00000101082 |
| <1e−07 | <1e−07 | 35.86 | 10.41 | 3.44 | WDR11- | 283089 | NR_033850 | Hs.568750 | ENSG00000227165 |
| | | | | | AS1 | | | | |
| <1e−07 | <1e−07 | 83.15 | 26.32 | 3.16 | ITGB5 | 3693 | NM_001354764 | Hs.13155 | ENSG00000082781 |
| <1e−07 | <1e−07 | 366.68 | 218.73 | 1.68 | PTPN12 | 5782 | NM_001131008 | Hs.61812 | ENSG00000127947 |
| <1e−07 | <1e−07 | 98.39 | 30.24 | 3.25 | ALOX12 | 239 | NM_000697 | Hs.654431 | ENSG00000108839 |
| <1e−07 | <1e−07 | 217.46 | 58.06 | 3.75 | H3C10 | 8357 | | | |
| <1e−07 | <1e−07 | 87.31 | 41.62 | 2.1 | MINDY1 | 55793 | NM_001040217 | Hs.743952 | ENSG00000143409 |
| <1e−07 | <1e−07 | 18.01 | 6.52 | 2.76 | AL731557.1 | | | | |
| <1e−07 | <1e−07 | 675.57 | 240.79 | 2.81 | CLU | 1191 | NM_001831 | Hs.436657 | ENSG00000120885 |
| <1e−07 | <1e−07 | 136.34 | 80.74 | 1.69 | SEPTIN6 | 23157 | | | |
| <1e−07 | <1e−07 | 35.77 | 11.31 | 3.16 | PCYT1B | 9468 | NM_001163264 | Hs.660708 | ENSG00000102230 |
| <1e−07 | <1e−07 | 28.35 | 12.15 | 2.33 | SCFD2 | 152579 | NM_152540 | Hs.302287 | ENSG00000184178 |
| <1e−07 | <1e−07 | 38.12 | 11.69 | 3.26 | P2RY12 | 64805 | NM_022788 | Hs.591281 | ENSG00000169313 |
| <1e−07 | <1e−07 | 319.35 | 96.37 | 3.31 | ITGB3 | 3690 | NM_000212 | Hs.218040 | ENSG00000259207 |
| <1e−07 | <1e−07 | 26.08 | 11.49 | 2.27 | TTC33 | 23548 | NM_012382 | Hs.348915 | ENSG00000113638 |
| <1e−07 | <1e−07 | 38.86 | 11.6 | 3.35 | FHL1 | 2273 | NM_001159699 | Hs.435369 | ENSG00000022267 |
| <1e−07 | <1e−07 | 55.3 | 28.26 | 1.96 | ZNF664 | 144348 | NM_001204298 | Hs.524828 | ENSG00000179195 |
| <1e−07 | <1e−07 | 242.33 | 73.17 | 3.31 | GP1BA | 2811 | NM_000173 | Hs.1472 | ENSG00000185245 |
| <1e−07 | <1e−07 | 377.76 | 181.49 | 2.08 | PIP4K2A | 5305 | NM_001330062 | Hs.57079 | ENSG00000150867 |
| <1e−07 | <1e−07 | 85.82 | 23.1 | 3.72 | ANK1 | 286 | NM_000037 | Hs.654438 | ENSG00000029534 |
| <1e−07 | <1e−07 | 47.24 | 14.23 | 3.32 | F2R | 2149 | NM_001311313 | Hs.482562 | ENSG00000181104 |
| <1e−07 | <1e−07 | 221.56 | 63.61 | 3.48 | DMTN | 2039 | NM_001114135 | Hs.106124 | ENSG00000158856 |
| <1e−07 | <1e−07 | 91.4 | 45.53 | 2.01 | NUTF2 | 10204 | NM_001322038 | Hs.356630 | ENSG00000102898 |
| <1e−07 | <1e−07 | 55.94 | 29.17 | 1.92 | VPS13A | 23230 | NM_001018037 | Hs.459790 | ENSG00000197969 |
| <1e−07 | <1e−07 | 20.21 | 7.22 | 2.8 | ZNF367 | 195828 | NM_153695 | Hs.494557 | ENSG00000165244 |
| <1e−07 | <1e−07 | 49.7 | 15.15 | 3.28 | CMTM5 | 116173 | NM_001037288 | Hs.99272 | ENSG00000166091 |
| <1e−07 | <1e−07 | 61.21 | 18.05 | 3.39 | CALD1 | 800 | NM_004342 | Hs.490203 | ENSG00000122786 |
| <1e−07 | <1e−07 | 48.41 | 13.36 | 3.62 | WHAMMP2 | 440253 | NR_026589 | Hs.558967 | |
| <1e−07 | <1e−07 | 59.56 | 34.08 | 1.75 | MTMR12 | 54545 | NM_001040446 | Hs.481836 | ENSG00000150712 |
| <1e−07 | <1e−07 | 21.7 | 7.63 | 2.84 | AP001636.3 | | | | |
| <1e−07 | <1e−07 | 79.51 | 24.01 | 3.31 | LINC00989 | 100506035 | NR_038826 | Hs.507664 | ENSG00000250334 |
| <1e−07 | <1e−07 | 119.65 | 55.5 | 2.16 | AGPAT1 | 10554 | NM_006411 | Hs.409230 | ENSG00000204310 |
| <1e−07 | <1e−07 | 21.26 | 7.73 | 2.75 | SENCR | 100507392 | NR_038908 | Hs.657715 | ENSG00000254703 |
| <1e−07 | <1e−07 | 438.9 | 233.67 | 1.88 | MYL12A | 10627 | NM_001303047 | Hs.190086 | ENSG00000101608 |
| <1e−07 | <1e−07 | 39.57 | 20.7 | 1.91 | RAB4A | 5867 | NM_001271998 | Hs.296169 | ENSG00000168118 |
| <1e−07 | <1e−07 | 22.12 | 7.98 | 2.77 | MSANTD3 | 91283 | NM_001198805 | Hs.530272 | ENSG00000066697 |
| <1e−07 | <1e−07 | 1662.12 | 562.73 | 2.95 | PPBP | 5473 | NM_002704 | Hs.2164 | ENSG00000163736 |
| <1e−07 | <1e−07 | 54.33 | 18.38 | 2.96 | C1orf198 | 84886 | NM_001136494 | Hs.520494 | ENSG00000119280 |
| <1e−07 | <1e−07 | 98.83 | 29.51 | 3.35 | PTCRA | 171558 | NM_001243168 | Hs.169002 | ENSG00000171611 |
| <1e−07 | <1e−07 | 33.23 | 17.6 | 1.89 | GTDC1 | 79712 | NM_001006636 | Hs.44780 | ENSG00000121964 |
| <1e−07 | <1e−07 | 32.08 | 10.16 | 3.16 | WASF3 | 10810 | NM_001291965 | Hs.618732 | ENSG00000132970 |
| <1e−07 | <1e−07 | 17.58 | 6.26 | 2.81 | C12orf75 | 387882 | NM_001145199 | Hs.368938 | ENSG00000235162 |
| <1e−07 | <1e−07 | 37.13 | 12.76 | 2.91 | TNFSF4 | 7292 | NM_001297562 | Hs.181097 | ENSG00000117586 |
| <1e−07 | <1e−07 | 73.85 | 23.79 | 3.1 | BMP6 | 654 | NM_001718 | Hs.285671 | ENSG00000153162 |
| <1e−07 | <1e−07 | 23.13 | 7.07 | 3.27 | AL445426.1 | | | | |
| <1e−07 | <1e−07 | 134.7 | 51.7 | 2.61 | EHD3 | 30845 | NM_014600 | Hs.368808 | ENSG00000013016 |
| <1e−07 | <1e−07 | 85.07 | 37.59 | 2.26 | NDUFAF3 | 25915 | NM_199069 | Hs.31387 | ENSG00000178057 |
| <1e−07 | <1e−07 | 16.61 | 6.99 | 2.38 | RTCA- | 100506007 | NR_110434 | Hs.192268 | |
| <1e−07 | <1e−07 | 72.85 | 23.23 | 3.14 | ESAM | 90952 | NM_138961 | Hs.173840 | ENSG00000149564 |
| <1e−07 | <1e−07 | 19.67 | 7.26 | 2.71 | LINC01011 | 401232 | NM_207495 | Hs.720158 | |
| <1e−07 | <1e−07 | 18.8 | 6.53 | 2.88 | GIPC3 | 126326 | NM_133261 | Hs.266873 | ENSG00000179855 |
| <1e−07 | <1e−07 | 155.04 | 85.76 | 1.81 | PPM1A | 5494 | NM_021003 | Hs.130036 | ENSG00000100614 |
| <1e−07 | <1e−07 | 83.96 | 33.59 | 2.5 | LDLRAP1 | 26119 | NM_015627 | Hs.590911 | ENSG00000157978 |
| <1e−07 | <1e−07 | 21.66 | 7.9 | 2.74 | Z82206.1 | | | | |
| <1e−07 | <1e−07 | 58.99 | 25.15 | 2.35 | TNIK | 23043 | NM_001161560 | Hs.34024 | ENSG00000154310 |
| <1e−07 | <1e−07 | 52.54 | 26.14 | 2.01 | NLK | 51701 | NM_016231 | Hs.208759 | ENSG00000087095 |
| <1e−07 | <1e−07 | 39.43 | 14.2 | 2.78 | TFPI | 7035 | NM_001032281 | Hs.516578 | ENSG00000003436 |
| <1e−07 | <1e−07 | 48.18 | 15.23 | 3.16 | CLEC1B | 51266 | NM_001099431 | Hs.409794 | ENSG00000165682 |
| <1e−07 | <1e−07 | 13.62 | 5.9 | 2.31 | SLC8A3 | 6547 | NM_001130417 | Hs.337696 | ENSG00000100678 |
| <1e−07 | <1e−07 | 94.46 | 55.37 | 1.71 | LEPROT | 54741 | NM_001198681 | Hs.23581 | ENSG00000213625 |
| <1e−07 | <1e−07 | 89.66 | 40.95 | 2.19 | RAB37 | 326624 | NM_001006637 | Hs.351413 | ENSG00000172794 |
| <1e−07 | <1e−07 | 113.07 | 66.73 | 1.69 | PCNP | 57092 | NM_001320395 | Hs.732633 | ENSG00000081154 |
| <1e−07 | <1e−07 | 134.67 | 79.8 | 1.69 | PTPRA | 5786 | NM_002836 | Hs.269577 | ENSG00000132670 |
| <1e−07 | <1e−07 | 29.17 | 13.19 | 2.21 | LYPLAL1 | 127018 | NM_001300769 | Hs.657617 | ENSG00000143353 |
| <1e−07 | <1e−07 | 335.34 | 182.96 | 1.83 | WBP2 | 23558 | NM_001330499 | Hs.514489 | ENSG00000132471 |
| <1e−07 | <1e−07 | 49.25 | 27.64 | 1.78 | LRRC8B | 23507 | NM_001134476 | Hs.482017 | ENSG00000197147 |
| <1e−07 | <1e−07 | 23.29 | 10.81 | 2.16 | TWSG1 | 57045 | NM_020648 | Hs.514685 | ENSG00000128791 |
| <1e−07 | <1e−07 | 17.68 | 6.72 | 2.63 | ZNF385D | 79750 | NM_024697 | Hs.21026 | ENSG00000151789 |
| <1e−07 | <1e−07 | 74.98 | 21.2 | 3.54 | ENKUR | 219670 | NM_001270383 | Hs.534486 | ENSG00000151023 |
| <1e−07 | <1e−07 | 89.99 | 45.21 | 1.99 | USP12 | 219333 | NM_182488 | Hs.42400 | ENSG00000152484 |
| <1e−07 | <1e−07 | 32.96 | 10.33 | 3.19 | TMCC2 | 9911 | NM_001242925 | Hs.6360 | ENSG00000133069 |
| <1e−07 | <1e−07 | 14.46 | 5.92 | 2.44 | TDRP | 157695 | NM_001256113 | Hs.289293 | ENSG00000180190 |
| <1e−07 | <1e−07 | 45.81 | 19.01 | 2.41 | SAV1 | 60485 | NM_021818 | Hs.642842 | ENSG00000151748 |
| <1e−07 | <1e−07 | 24.08 | 8.6 | 2.8 | LPAR5 | 57121 | NM_001142961 | Hs.155538 | ENSG00000184574 |
| <1e−07 | <1e−07 | 20.62 | 7.86 | 2.62 | GGTA1P | 2681 | NR_003191 | Hs.97469 | ENSG00000204136 |
| <1e−07 | <1e−07 | 27.33 | 10.83 | 2.52 | NFATC2 | 4773 | NM_001136021 | Hs.744148 | ENSG00000101096 |
| <1e−07 | <1e−07 | 21.07 | 7.33 | 2.88 | AC147651.1 | | | | |
| <1e−07 | <1e−07 | 57.36 | 16.68 | 3.44 | SPTB | 6710 | NM_000347 | Hs.417303 | ENSG00000070182 |
| <1e−07 | <1e−07 | 15.57 | 6.12 | 2.55 | GNAO1 | 2775 | NM_020988 | Hs.644524 | ENSG00000087258 |
| <1e−07 | <1e−07 | 63.27 | 15.81 | 4 | CXCL5 | 6374 | NM_002994 | Hs.89714 | ENSG00000163735 |
| <1e−07 | <1e−07 | 52.27 | 19.54 | 2.68 | H2BC7 | 8343 | | | |
| <1e−07 | <1e−07 | 661.76 | 374.94 | 1.76 | NORAD | 647979 | NR_027451 | Hs.743232 | ENSG00000260032 |
| <1e−07 | <1e−07 | 20.03 | 7.4 | 2.71 | PDE3A | 5139 | NM_000921 | Hs.386791 | ENSG00000172572 |
| <1e−07 | <1e−07 | 121.93 | 67.09 | 1.82 | CLCN3 | 1182 | NM_001243372 | Hs.481186 | ENSG00000109572 |
| <1e−07 | <1e−07 | 13.04 | 6.94 | 1.88 | MTMR2 | 8898 | NM_001243571 | Hs.181326 | ENSG00000087053 |
| <1e−07 | <1e−07 | 125.44 | 71.54 | 1.7 | PCMTD1 | 115294 | NM_001286782 | Hs.671268 | ENSG00000168300 |
| <1e−07 | <1e−07 | 94.04 | 33.28 | 2.83 | PITPNM2 | 57605 | NM_001300801 | Hs.272759 | ENSG00000090975 |
| <1e−07 | <1e−07 | 48.21 | 18.82 | 2.56 | SSX2IP | 117178 | NM_001166293 | Hs.22587 | ENSG00000117155 |
| <1e−07 | <1e−07 | 38.53 | 14.58 | 2.64 | PDGFA | 5154 | NM_002607 | Hs.535898 | ENSG00000197461 |
| <1e−07 | <1e−07 | 134.3 | 54.86 | 2.45 | BCL2L1 | 598 | NM_001191 | Hs.516966 | ENSG00000171552 |
| <1e−07 | <1e−07 | 61.61 | 18.43 | 3.34 | SNCA | 6622 | NM_000345 | Hs.21374 | ENSG00000145335 |
| <1e−07 | <1e−07 | 457.28 | 244.71 | 1.87 | SH3BGRL | 6451 | NM_003022 | Hs.108029 | ENSG00000131171 |
| <1e−07 | <1e−07 | 67.33 | 30.84 | 2.18 | SMIM3 | 85027 | NM_032947 | Hs.29444 | ENSG00000256235 |
| <1e−07 | <1e−07 | 46.2 | 18.86 | 2.45 | ISCA1 | 81689 | NM_030940 | Hs.449291 | ENSG00000135070 |
| <1e−07 | <1e−07 | 1073.98 | 648.2 | 1.66 | MBNL1 | 4154 | NM_001314057 | Hs.201858 | ENSG00000152601 |
| <1e−07 | <1e−07 | 3011.7 | 1489.98 | 2.02 | TMSB4X | 7114 | NM_021109 | Hs.437277 | ENSG00000205542 |
| <1e−07 | <1e−07 | 168.42 | 100.74 | 1.67 | AMD1 | 262 | NM_001033059 | Hs.159118 | ENSG00000123505 |
| <1e−07 | <1e−07 | 55.63 | 29.03 | 1.92 | NDUFA6 | 4700 | NM_002490 | Hs.274416 | ENSG00000184983 |
| <1e−07 | <1e−07 | 42.53 | 14.58 | 2.92 | H2BC9 | 8345 | | | |
| <1e−07 | <1e−07 | 21.59 | 8.86 | 2.44 | CCDC7 | 79741 | NM_001026383 | Hs.585464 | ENSG00000216937 |
| <1e−07 | <1e−07 | 78.26 | 29.11 | 2.69 | SMOX | 54498 | NM_001270691 | Hs.433337 | ENSG00000088826 |
| <1e−07 | <1e−07 | 15.77 | 6.22 | 2.53 | SH3TC2 | 79628 | NM_024577 | Hs.483784 | ENSG00000169247 |
| <1e−07 | <1e−07 | 17.65 | 6.63 | 2.66 | NT5M | 56953 | NM_020201 | Hs.513977 | ENSG00000205309 |
| <1e−07 | <1e−07 | 88.2 | 24.75 | 3.56 | JAM3 | 83700 | NM_001205329 | Hs.150718 | ENSG00000166086 |
| <1e−07 | <1e−07 | 42.26 | 13.4 | 3.15 | MPL | 4352 | NM_005373 | Hs.82906 | ENSG00000117400 |
| <1e−07 | <1e−07 | 34.05 | 15.82 | 2.15 | SLC10A3 | 8273 | NM_001142391 | Hs.522826 | ENSG00000126903 |
| <1e−07 | <1e−07 | 10.89 | 5.33 | 2.04 | PKIG | 11142 | NM_001281444 | Hs.472831 | ENSG00000168734 |
| <1e−07 | <1e−07 | 72.8 | 24.6 | 2.96 | ABCC4 | 10257 | NM_001105515 | Hs.508423 | ENSG00000125257 |
| <1e−07 | <1e−07 | 117.25 | 64.11 | 1.83 | CLIC4 | 25932 | NM_013943 | Hs.440544 | ENSG00000169504 |
| <1e−07 | <1e−07 | 597.44 | 342.76 | 1.74 | CALM3 | 808 | NM_001329921 | Hs.515487 | ENSG00000160014 |
| <1e−07 | <1e−07 | 13.01 | 6.1 | 2.13 | TMSB4XP8 | 7117 | NM_183049 | | |
| <1e−07 | <1e−07 | 85.89 | 27.64 | 3.11 | MMRN1 | 22915 | NM_007351 | Hs.268107 | ENSG00000138722 |
| <1e−07 | <1e−07 | 78.33 | 50.23 | 1.56 | XPNPEP1 | 7511 | NM_001167604 | Hs.390623 | ENSG00000108039 |
| <1e−07 | <1e−07 | 1929.35 | 3283.68 | 0.59 | FCN1 | 2219 | NM_002003 | Hs.440898 | ENSG00000085265 |
| <1e−07 | <1e−07 | 22.48 | 11.21 | 2.01 | CD27-AS1 | 678655 | NR_015382 | Hs.655224 | ENSG00000215039 |
| <1e−07 | <1e−07 | 138.33 | 88.34 | 1.57 | NPTN | 27020 | NM_001161363 | Hs.744867 | ENSG00000156642 |
| <1e−07 | <1e−07 | 154.15 | 39.97 | 3.86 | AP000547.3 | | | | |
| <1e−07 | <1e−07 | 24.89 | 8.65 | 2.88 | RNF208 | 727800 | NM_031297 | Hs.512767 | ENSG00000212864 |
| <1e−07 | <1e−07 | 40.8 | 12.31 | 3.31 | HEMGN | 55363 | NM_018437 | Hs.176626 | ENSG00000136929 |
| <1e−07 | <1e−07 | 84.56 | 44.24 | 1.91 | EMC3 | 55831 | NM_018447 | Hs.475392 | ENSG00000125037 |
| <1e−07 | <1e−07 | 32.91 | 17.83 | 1.85 | DAAM1 | 23002 | NM_001270520 | Hs.19156 | ENSG00000100592 |
| <1e−07 | <1e−07 | 17.12 | 6.74 | 2.54 | LANCL3 | 347404 | NM_001170331 | Hs.521932 | ENSG00000147036 |
| <1e−07 | <1e−07 | 299.45 | 466.06 | 0.64 | ITGAM | 3684 | NM_000632 | Hs.172631 | ENSG00000169896 |
| <1e−07 | <1e−07 | 101.81 | 32.0 | 3.18 | AP003068.2 | | | | |
| <1e−07 | <1e−07 | 39.34 | 15.82 | 2.49 | MAST4 | 375449 | NM_001164664 | Hs.595458 | ENSG00000069020 |
| <1e−07 | <1e−07 | 13.09 | 5.69 | 2.3 | EFHC2 | 80258 | NM_025184 | Hs.521953 | ENSG00000183690 |
| <1e−07 | <1e−07 | 226.84 | 109.49 | 2.07 | PPDPF | 79144 | NM_001353423 | Hs.79625 | ENSG00000125534 |
| <1e−07 | <1e−07 | 33.48 | 10.21 | 3.28 | RBPMS2 | 348093 | NM_194272 | Hs.436518 | ENSG00000166831 |
| <1e−07 | <1e−07 | 14.87 | 6.52 | 2.28 | MSRB3 | 253827 | NM_001031679 | Hs.339024 | ENSG00000174099 |
| <1e−07 | <1e−07 | 91.31 | 41.56 | 2.2 | ABCC3 | 8714 | NM_001144070 | Hs.463421 | ENSG00000108846 |
| <1e−07 | <1e−07 | 21.17 | 8.47 | 2.5 | SLFN14 | 342618 | NM_001129820 | Hs.591193 | ENSG00000236320 |
| <1e−07 | <1e−07 | 14.2 | 6.17 | 2.3 | ACER2 | 340485 | NM_001010887 | Hs.41379 | ENSG00000177076 |
| <1e−07 | <1e−07 | 13.22 | 6.17 | 2.14 | TMEM64 | 169200 | NM_001008495 | Hs.567759 | ENSG00000180694 |
| <1e−07 | <1e−07 | 38.48 | 21.45 | 1.79 | ZNF271P | 10778 | NM_006629 | Hs.314246 | ENSG00000257267 |
| <1e−07 | <1e−07 | 39.68 | 14.29 | 2.78 | TSPAN9 | 10867 | NM_001168320 | Hs.504517 | ENSG00000011105 |
| <1e−07 | <1e−07 | 25.73 | 8.37 | 3.07 | SEC14L5 | 9717 | NM_014692 | Hs.512856 | ENSG00000103184 |
| <1e−07 | <1e−07 | 35.79 | 10.71 | 3.34 | GMPR | 2766 | NM_006877 | Hs.484741 | ENSG00000137198 |
| <1e−07 | <1e−07 | 188.84 | 106.94 | 1.77 | RABGAP1L | 9910 | NM_001035230 | Hs.585378 | ENSG00000152061 |
| <1e−07 | <1e−07 | 12.98 | 5.84 | 2.22 | AC107223.1 | | | | |
| <1e−07 | <1e−07 | 63.53 | 33.83 | 1.88 | UXS1 | 80146 | NM_001253875 | Hs.469561 | ENSG00000115652 |
| <1e−07 | <1e−07 | 52.03 | 25.83 | 2.01 | VDAC3 | 7419 | NM_001135694 | Hs.699301 | ENSG00000078668 |
| <1e−07 | <1e−07 | 19.66 | 7.24 | 2.72 | CTSW | 1521 | NM_001335 | Hs.416848 | ENSG00000172543 |
| <1e−07 | <1e−07 | 13.33 | 6.09 | 2.19 | CDKL1 | 8814 | NM_001282236 | Hs.280881 | ENSG00000100490 |
| <1e−07 | <1e−07 | 18.44 | 7.26 | 2.54 | CABP5 | 56344 | NM_016367 | Hs.117694 | ENSG00000105507 |
| <1e−07 | <1e−07 | 116.24 | 39.61 | 2.93 | CD9 | 928 | NM_001330312 | Hs.114286 | ENSG00000010278 |
| <1e−07 | <1e−07 | 73.42 | 28.59 | 2.57 | TMEM140 | 55281 | NM_018295 | Hs.521213 | ENSG00000146859 |
| <1e−07 | <1e−07 | 16.11 | 6.75 | 2.39 | PLOD2 | 5352 | NM_000935 | Hs.477866 | ENSG00000152952 |
| <1e−07 | <1e−07 | 175.67 | 90.59 | 1.94 | SNAP23 | 8773 | NM_003825 | Hs.511149 | ENSG00000092531 |
| <1e−07 | <1e−07 | 25.31 | 11.73 | 2.16 | APLF | 200558 | NM_173545 | Hs.720369 | ENSG00000169621 |
| <1e−07 | <1e−07 | 348.71 | 232.16 | 1.5 | APP | 351 | NM_000484 | Hs.434980 | ENSG00000142192 |
| <1e−07 | <1e−07 | 27.6 | 13.21 | 2.09 | ARHGAP10 | 79658 | NM_024605 | Hs.368631 | ENSG00000071205 |
| <1e−07 | <1e−07 | 36.71 | 19.43 | 1.89 | AKT3 | 10000 | NM_001206729 | Hs.498292 | ENSG00000117020 |
| <1e−07 | <1e−07 | 41.47 | 23.79 | 1.74 | NDUFA5 | 4698 | NM_001282419 | Hs.651219 | ENSG00000128609 |
| <1e−07 | <1e−07 | 18.75 | 9.25 | 2.03 | TRAPPC2 | 6399 | NM_001011658 | Hs.592238 | ENSG00000196459 |
| <1e−07 | <1e−07 | 484.22 | 255.41 | 1.9 | TAGLN2 | 8407 | NM_001277223 | Hs.517168 | ENSG00000158710 |
| <1e−07 | <1e−07 | 27.21 | 13.66 | 1.99 | PDGFC | 56034 | NM_016205 | Hs.570855 | ENSG00000145431 |
| <1e−07 | <1e−07 | 17.87 | 8.29 | 2.15 | LINC02284 | 102723699 | XR 001750778 | | |
| <1e−07 | <1e−07 | 18.28 | 7.31 | 2.5 | AVPR1A | 552 | NM_000706 | Hs.2131 | ENSG00000166148 |
| <1e−07 | <1e−07 | 12.83 | 5.75 | 2.23 | SMIM5 | 643008 | NM_001162995 | Hs.528605 | ENSG00000204323 |
| <1e−07 | <1e−07 | 14.88 | 6.43 | 2.31 | RTN2 | 6253 | NM_005619 | Hs.47517 | ENSG00000125744 |
| <1e−07 | <1e−07 | 38.87 | 18.48 | 2.1 | UBL4A | 8266 | NM_014235 | Hs.76480 | ENSG00000102178 |
| <1e−07 | <1e−07 | 293.3 | 114.98 | 2.55 | RSU1 | 6251 | NM_012425 | Hs.524161 | ENSG00000148484 |
| <1e−07 | <1e−07 | 16.54 | 8.11 | 2.04 | OPHN1 | 4983 | NM_002547 | Hs.128824 | ENSG00000079482 |
| <1e−07 | <1e−07 | 94.03 | 35.07 | 2.68 | MGLL | 11343 | NM_001003794 | Hs.277035 | ENSG00000074416 |
| <1e−07 | <1e−07 | 21.02 | 8.05 | 2.61 | HOMER2 | 9455 | NM_004839 | Hs.578443 | ENSG00000103942 |
| <1e−07 | <1e−07 | 49.85 | 15.52 | 3.21 | PROS1 | 5627 | NM_000313 | Hs.64016 | ENSG00000184500 |
| <1e−07 | <1e−07 | 102.37 | 37.88 | 2.7 | MAP1A | 4130 | NM_002373 | Hs.194301 | ENSG00000166963 |
| <1e−07 | <1e−07 | 35.83 | 21.13 | 1.7 | RBX1 | 9978 | NM_014248 | Hs.474949 | ENSG00000100387 |
| <1e−07 | <1e−07 | 10.43 | 5.32 | 1.96 | INKA2- | 100506343 | | | |
| | | | | | AS1 | | | | |
| <1e−07 | <1e−07 | 49.24 | 28.8 | 1.71 | RHBDD1 | 84236 | NM_001167608 | Hs.471514 | ENSG00000144468 |
| <1e−07 | <1e−07 | 19.55 | 9.17 | 2.13 | KIFAP3 | 22920 | NM_001204514 | Hs.433442 | ENSG00000075945 |
| <1e−07 | <1e−07 | 15.59 | 6.23 | 2.5 | AL133444.1 | | | | |
| <1e−07 | <1e−07 | 51.72 | 24.43 | 2.12 | CDK2AP1 | 8099 | NM_001270433 | Hs.433201 | ENSG00000111328 |
| <1e−07 | <1e−07 | 132.12 | 87.78 | 1.51 | KCTD20 | 222658 | NM_001286579 | Hs.188757 | ENSG00000112078 |
| <1e−07 | <1e−07 | 13.52 | 5.86 | 2.31 | MFSD2B | 388931 | NM_001080473 | Hs.407482 | ENSG00000205639 |
| <1e−07 | <1e−07 | 11.68 | 5.41 | 2.16 | AL355073.1 | | | | |
| <1e−07 | <1e−07 | 15.48 | 7.47 | 2.07 | AGBL5 | 60509 | NM_001035507 | Hs.138207 | ENSG00000084693 |
| <1e−07 | <1e−07 | 24.36 | 9.36 | 2.6 | H2BC8 | 8339 | | | |
| <1e−07 | <1e−07 | 184.38 | 120.6 | 1.53 | TRAM1 | 23471 | NM_001317804 | Hs.491988 | ENSG00000067167 |
| <1e−07 | <1e−07 | 44.2 | 25.87 | 1.71 | SLC37A1 | 54020 | NM_001320537 | Hs.547009 | ENSG00000160190 |
| <1e−07 | <1e−07 | 22.09 | 9.21 | 2.4 | LEPR | 3953 | NM_001003679 | Hs.23581 | ENSG00000116678 |
| <1e−07 | <1e−07 | 433.4 | 257.51 | 1.68 | CTSA | 5476 | NM_000308 | Hs.609336 | ENSG00000064601 |
| <1e−07 | <1e−07 | 358.44 | 182.63 | 1.96 | H2BC21 | 8349 | | | |
| <1e−07 | <1e−07 | 19.46 | 9.47 | 2.06 | ST7 | 7982 | NM_018412 | Hs.368131 | ENSG00000004866 |
| <1e−07 | <1e−07 | 64.96 | 23.36 | 2.78 | TSPAN18 | 90139 | NM_001031730 | Hs.385634 | ENSG00000157570 |
| <1e−07 | <1e−07 | 96.79 | 49.51 | 1.96 | H2BC4 | 8347 | | | |
| <1e−07 | <1e−07 | 98.16 | 43.27 | 2.27 | MARCHF2 | 51257 | | | |
| <1e−07 | <1e−07 | 12.18 | 6.07 | 2.01 | LRP12 | 29967 | NM_001135703 | Hs.600630 | ENSG00000147650 |
| <1e−07 | <1e−07 | 157.42 | 101.1 | 1.56 | PAIP2 | 51247 | NM_001033112 | Hs.396644 | ENSG00000120727 |
| <1e−07 | <1e−07 | 79.72 | 134.19 | 0.59 | FES | 2242 | NM_001143783 | Hs.7636 | ENSG00000182511 |
| <1e−07 | <1e−07 | 39.39 | 19.52 | 2.02 | CDIP1 | 29965 | NM_001199054 | Hs.654653 | ENSG00000089486 |
| <1e−07 | <1e−07 | 121.99 | 73.88 | 1.65 | DAP | 1611 | NM_001291963 | Hs.75189 | ENSG00000112977 |
| <1e−07 | <1e−07 | 38.98 | 21.33 | 1.83 | DAD1 | 1603 | NM_001344 | Hs.82890 | ENSG00000129562 |
| <1e−07 | <1e−07 | 42.6 | 24.55 | 1.74 | AC005332.6 | | | | |
| <1e−07 | <1e−07 | 36.09 | 12.93 | 2.79 | SYNM | 23336 | NM_015286 | Hs.207106 | ENSG00000182253 |
| <1e−07 | <1e−07 | 13.72 | 6.3 | 2.18 | TMEM185A | 84548 | NM_001174092 | Hs.522172 | ENSG00000269556 |
| <1e−07 | <1e−07 | 14.88 | 7.63 | 1.95 | NENF | 29937 | NM_013349 | Hs.461787 | ENSG00000117691 |
| <1e−07 | <1e−07 | 48.35 | 16.6 | 2.91 | ABLIM1 | 3983 | NM_001003407 | Hs.438236 | ENSG00000099204 |
| <1e−07 | <1e−07 | 21.46 | 8.11 | 2.65 | SLC24A3 | 57419 | NM_020689 | Hs.654790 | ENSG00000185052 |
| <1e−07 | <1e−07 | 12.21 | 5.31 | 2.3 | ADRA2A | 150 | NM_000681 | Hs.249159 | ENSG00000150594 |
| <1e−07 | <1e−07 | 27.03 | 13.73 | 1.97 | ABI2 | 10152 | NM_001282925 | Hs.471156 | ENSG00000138443 |
| <1e−07 | <1e−07 | 16.8 | 8 | 2.1 | GCLM | 2730 | NM_001308253 | Hs.315562 | ENSG0000002390 |
| <1e−07 | <1e−07 | 405.88 | 244.88 | 1.66 | ACTN1 | 87 | NM_001102 | Hs.235750 | ENSG00000072110 |
| <1e−07 | <1e−07 | 1272.34 | 788.6 | 1.61 | SH3BGRL3 | 83442 | NM_031286 | Hs.109051 | ENSG00000142669 |
| <1e−07 | <1e−07 | 120.17 | 53.5 | 2.25 | TSPAN33 | 340348 | NM_178562 | Hs.27267 | ENSG00000158457 |
| <1e−07 | <1e−07 | 160.46 | 70.39 | 2.28 | SLC40A1 | 30061 | NM_014585 | Hs.643005 | ENSG00000138449 |
| <1e−07 | <1e−07 | 161.36 | 78.74 | 2.05 | DAPP1 | 27071 | NM_001306151 | Hs.436271 | ENSG00000070190 |
| <1e−07 | <1e−07 | 73.71 | 37.92 | 1.94 | TUBA1C | 84790 | NM_001303114 | Hs.652390 | ENSG00000167553 |
| <1e−07 | <1e−07 | 31.78 | 17.65 | 1.8 | ZCCHC17 | 51538 | NM_001282566 | Hs.524094 | ENSG00000121766 |
| <1e−07 | <1e−07 | 138.81 | 89.39 | 1.55 | TMBIM1 | 64114 | NM_001321427 | Hs.591605 | ENSG00000135926 |
| <1e−07 | <1e−07 | 10.97 | 5.07 | 2.16 | CFAP161 | 161502 | NM_001353365 | Hs.130979 | ENSG00000156206 |
| <1e−07 | <1e−07 | 52.55 | 26.45 | 1.99 | PDCD10 | 11235 | NM_007217 | Hs.478150 | ENSG00000114209 |
| <1e−07 | <1e−07 | 131.37 | 84.69 | 1.55 | SLAIN2 | 57606 | NM_020846 | Hs.479677 | ENSG00000109171 |
| <1e−07 | <1e−07 | 21.94 | 7.78 | 2.82 | TGFB1I1 | 7041 | NM_001042454 | Hs.513530 | ENSG00000140682 |
| <1e−07 | <1e−07 | 35.27 | 19.59 | 1.8 | MIR4435- | 541471 | NR_015395 | Hs.560805 | ENSG00000172965 |
| | | | | | 2HG | | | | |
| <1e−07 | <1e−07 | 118.78 | 77.57 | 1.53 | YWHAQ | 10971 | NM_006826 | Hs.74405 | ENSG00000134308 |
| <1e−07 | <1e−07 | 45.17 | 20 | 2.26 | H2AC8 | 3012 | | | |
| <1e−07 | <1e−07 | 46.66 | 18.48 | 2.53 | ANKRD33B | 651746 | NM_001164440 | Hs.26039 | ENSG00000164236 |
| <1e−07 | <1e−07 | 89.38 | 42.69 | 2.09 | STMP1 | 647087 | NM_001130929 | Hs.200022 | ENSG00000243317 |
| <1e−07 | <1e−07 | 107.33 | 66.67 | 1.61 | PPP3R1 | 5534 | NM_000945 | Hs.280604 | ENSG00000221823 |
| <1e−07 | <1e−07 | 54.01 | 25.62 | 2.11 | FRMD3 | 257019 | NM_001244959 | Hs.127535 | ENSG00000172159 |
| <1e−07 | <1e−07 | 29.04 | 15.55 | 1.87 | ZFAND1 | 79752 | NM_001170796 | Hs.655453 | ENSG00000104231 |
| <1e−07 | <1e−07 | 10.54 | 5.67 | 1.86 | AP001000.1 | | | | |
| <1e−07 | <1e−07 | 14.1 | 5.78 | 2.44 | NFIB | 4781 | NM_001190737 | Hs.644095 | ENSG00000147862 |
| <1e−07 | <1e−07 | 22.31 | 9.58 | 2.33 | TPTEP1 | 387590 | NR_001591 | Hs.474116 | ENSG00000100181 |
| <1e−07 | <1e−07 | 21.91 | 8.52 | 2.57 | SYTL4 | 94121 | NM_001129896 | Hs.592224 | ENSG00000102362 |
| <1e−07 | <1e−07 | 75.94 | 41.86 | 1.81 | RDH11 | 51109 | NM_001252650 | Hs.719925 | ENSG00000072042 |
| <1e−07 | <1e−07 | 23.48 | 8.61 | 2.73 | SPOCD1 | 90853 | NM_001281987 | Hs.62604 | ENSG00000134668 |
| <1e−07 | <1e−07 | 26.46 | 13.77 | 1.92 | ARL15 | 54622 | NM_019087 | Hs.659125 | ENSG00000185305 |
| <1e−07 | <1e−07 | 16.07 | 8.16 | 1.97 | EPOR | 2057 | NM_000121 | Hs.631624 | ENSG00000187266 |
| <1e−07 | <1e−07 | 201.95 | 119.26 | 1.69 | RIOK3 | 8780 | NM_001348193 | Hs.445511 | ENSG00000101782 |
| <1e−07 | <1e−07 | 354.94 | 128.92 | 2.75 | MAP3K7CL | 56911 | NM_001286617 | Hs.222802 | ENSG00000156265 |
| <1e−07 | <1e−07 | 44.25 | 69.68 | 0.64 | SLC4A2 | 6522 | NM_001199692 | Hs.647069 | ENSG00000164889 |
| <1e−07 | <1e−07 | 425.48 | 653.29 | 0.65 | FGR | 2268 | NM_001042729 | Hs.1422 | ENSG00000000938 |
| <1e−07 | <1e−07 | 8.99 | 5.06 | 1.77 | CCNJL | 79616 | NM_001308173 | Hs.14070 | ENSG00000135083 |
| <1e−07 | <1e−07 | 13.93 | 6.19 | 2.25 | COL24A1 | 255631 | NM_001349955 | Hs.659516 | ENSG00000171502 |
| <1e−07 | <1e−07 | 27.73 | 15.28 | 1.81 | MDM1 | 56890 | NM_001205028 | Hs.655702 | ENSG00000111554 |
| <1e−07 | <1e−07 | 26.49 | 12.08 | 2.19 | KIF3C | 3797 | NM_002254 | Hs.21611 | ENSG00000084731 |
| <1e−07 | <1e−07 | 41.73 | 66.13 | 0.63 | MLLT1 | 4298 | NM_005934 | Hs.10095 | ENSG00000130382 |
| <1e−07 | <1e−07 | 185.64 | 286.31 | 0.65 | STAT6 | 6778 | NM_001178078 | Hs.524518 | ENSG00000166888 |
| <1e−07 | <1e−07 | 35.36 | 13.52 | 2.62 | TBXA2R | 6915 | NM_001060 | Hs.442530 | ENSG00000006638 |
| <1e−07 | <1e−07 | 85.37 | 52.71 | 1.62 | TUBB4B | 10383 | NM_006088 | Hs.433615 | ENSG00000188229 |
| <1e−07 | <1e−07 | 1219.53 | 1994.17 | 0.61 | LRP1 | 4035 | NM_002332 | Hs.162757 | ENSG00000123384 |
| <1e−07 | <1e−07 | 222.49 | 358.57 | 0.62 | ARHGAP30 | 257106 | NM_001025598 | Hs.389374 | ENSG00000186517 |
| <1e−07 | <1e−07 | 99.82 | 62.31 | 1.6 | CARD19 | 84270 | NM_001318010 | Hs.434213 | ENSG00000165233 |
| <1e−07 | <1e−07 | 28.78 | 11.59 | 2.48 | AIG1 | 51390 | NM_001286587 | Hs.567501 | ENSG00000146416 |
| <1e−07 | <1e−07 | 73.99 | 40.99 | 1.8 | INAFM2 | 100505573 | NM_001301268 | Hs.530791 | ENSG00000259330 |
| <1e−07 | <1e−07 | 24.6 | 8.63 | 2.85 | PRKAR1B | 5575 | NM_001164758 | Hs.520851 | ENSG00000188191 |
| <1e−07 | <1e−07 | 40.04 | 26.35 | 1.52 | ATP8A1 | 10396 | NM_001105529 | Hs.435052 | ENSG00000124406 |
| <1e−07 | <1e−07 | 13.81 | 6.57 | 2.1 | MYOM1 | 8736 | NM_003803 | Hs.464469 | ENSG00000101605 |
| <1e−07 | <1e−07 | 66.9 | 39.1 | 1.71 | PRKCA | 5578 | NM_002737 | Hs.531704 | ENSG00000154229 |
| <1e−07 | <1e−07 | 224.29 | 63.9 | 3.51 | AP001189.1 | | | | |
| <1e−07 | <1e−07 | 66.37 | 108.37 | 0.61 | NACC2 | 138151 | NM_144653 | Hs.112895 | ENSG00000148411 |
| <1e−07 | <1e−07 | 12.52 | 5.87 | 2.13 | AFAP1L2 | 84632 | NM_001001936 | Hs.501106 | ENSG00000169129 |
| <1e−07 | <1e−07 | 1049.47 | 1642.42 | 0.64 | ITGB2 | 3689 | NM_000211 | Hs.375957 | ENSG00000160255 |
| <1e−07 | <1e−07 | 12.64 | 5.92 | 2.13 | SLC6A4 | 6532 | NM_001045 | Hs.29792 | ENSG00000108576 |
| <1e−07 | <1e−07 | 281.53 | 136.25 | 2.07 | RUFY1 | 80230 | NM_001040451 | Hs.306769 | ENSG00000176783 |
| <1e−07 | <1e−07 | 138.33 | 223.23 | 0.62 | SUN2 | 25777 | NM_001199579 | Hs.517622 | ENSG00000100242 |
| <1e−07 | <1e−07 | 21.7 | 11.95 | 1.82 | CAMTA1 | 23261 | NM_001195563 | Hs.397705 | ENSG00000171735 |
| <1e−07 | <1e−07 | 92.71 | 29 | 3.2 | GP9 | 2815 | NM_000174 | Hs.1144 | ENSG00000169704 |
| <1e−07 | <1e−07 | 17.03 | 7.34 | 2.32 | PSD3 | 23362 | NM_015310 | Hs.434255 | ENSG00000156011 |
| <1e−07 | <1e−07 | 11.39 | 5.62 | 2.03 | MISP3 | 113230 | NM_001291291 | Hs.372775 | ENSG00000141854 |
| <1e−07 | <1e−07 | 101.91 | 62.5 | 1.63 | UBE2H | 7328 | NM_001202498 | Hs.643548 | ENSG00000186591 |
| <1e−07 | <1e−07 | 119.71 | 57.74 | 2.07 | YWHAH | 7533 | NM_003405 | Hs.226755 | ENSG00000128245 |
| <1e−07 | <1e−07 | 21.37 | 7.41 | 2.88 | TMEM158 | 25907 | NM_015444 | Hs.740403 | ENSG00000249992 |
| <1e−07 | <1e−07 | 13.05 | 5.93 | 2.2 | INPP4B | 8821 | NM_001101669 | Hs.176376 | ENSG00000109452 |
| <1e−07 | <1e−07 | 62 | 26.09 | 2.38 | PNMA1 | 9240 | NM_006029 | Hs.194709 | ENSG00000176903 |
| <1e−07 | <1e−07 | 21.62 | 8.67 | 2.49 | ZNF542P | 147947 | NM_194319 | Hs.467326 | ENSG00000240225 |
| <1e−07 | <1e−07 | 1596.27 | 676.93 | 2.36 | MT-ND6 | 4541 | | | |
| <1e−07 | <1e−07 | 224.85 | 87.13 | 2.58 | MT-TP | 4571 | | | |
| <1e−07 | <1e−07 | 55.18 | 28.63 | 1.93 | GRK5 | 2869 | NM_005308 | Hs.524625 | ENSG00000198873 |
| <1e−07 | <1e−07 | 8.86 | 5.37 | 1.65 | ABCA3 | 21 | NM_001089 | Hs.26630 | ENSG00000167972 |
| <1e−07 | <1e−07 | 32.37 | 12.74 | 2.54 | PTPRS | 5802 | NM_002850 | Hs.744928 | ENSG00000105426 |
| <1e−07 | <1e−07 | 10.97 | 5.76 | 1.9 | CCR4 | 1233 | NM_005508 | Hs.184926 | ENSG00000183813 |
| <1e−07 | <1e−07 | 269.76 | 149.95 | 1.8 | SNN | 8303 | NM_003498 | Hs.618526 | ENSG00000184602 |
| <1e−07 | <1e−07 | 9.44 | 5.29 | 1.78 | PRKCQ | 5588 | NM_001242413 | Hs.498570 | ENSG00000065675 |
| <1e−07 | <1e−07 | 34.39 | 19.68 | 1.75 | CCNG1 | 900 | NM_004060 | Hs.79101 | ENSG00000113328 |
| <1e−07 | <1e−07 | 305.3 | 467.12 | 0.65 | AKNA | 80709 | NM_001317950 | Hs.494895 | ENSG00000106948 |
| <1e−07 | <1e−07 | 19.41 | 11.11 | 1.75 | AK3 | 50808 | NM_001199852 | Hs.732022 | ENSG00000147853 |
| <1e−07 | <1e−07 | 21.86 | 12.76 | 1.71 | NREP | 9315 | NM_001142474 | Hs.36053 | ENSG00000134986 |
| <1e−07 | <1e−07 | 48.79 | 75.92 | 0.64 | NCF4 | 4689 | NM_000631 | Hs.474781 | ENSG00000100365 |
| <1e−07 | <1e−07 | 18.16 | 7.1 | 2.56 | AQP10 | 89872 | NM_080429 | Hs.259048 | ENSG00000143595 |
| <1e−07 | <1e−07 | 22.11 | 11.26 | 1.96 | USP31 | 57478 | NM_020718 | Hs.183817 | ENSG00000103404 |
| <1e−07 | <1e−07 | 55.82 | 33.06 | 1.69 | PIP4P2 | 55529 | NM_018710 | Hs.202517 | ENSG00000155099 |
| <1e−07 | <1e−07 | 35.98 | 21 | 1.71 | ATE1 | 11101 | NM_001001976 | Hs.632080 | ENSG00000107669 |
| <1e−07 | <1e−07 | 11.27 | 5.49 | 2.05 | MCPH1- | 100507530 | NR_125386 | Hs.490892 | |
| | | | | | AS1 | | | | |
| <1e−07 | <1e−07 | 36.97 | 12.24 | 3.02 | SAMD14 | 201191 | NM_001257359 | Hs.567769 | ENSG00000167100 |
| <1e−07 | <1e−07 | 207.47 | 342.91 | 0.61 | NFAM1 | 150372 | NM_001318323 | Hs.436677 | ENSG00000235568 |
| <1e−07 | <1e−07 | 77.15 | 40.48 | 1.91 | SIAH2 | 6478 | NM_005067 | Hs.477959 | ENSG00000181788 |
| <1e−07 | <1e−07 | 68.05 | 36.44 | 1.87 | TRAPPC1 | 58485 | NM_001166621 | Hs.24379 | ENSG00000170043 |
| <1e−07 | <1e−07 | 8.98 | 5.46 | 1.64 | ZBED5- | 729013 | NR_034137 | Hs.726427 | ENSG00000247271 |
| | | | | | AS1 | | | | |
| <1e−07 | <1e−07 | 10.84 | 5.96 | 1.82 | SOX12 | 6666 | NM_006943 | Hs.43627 | ENSG00000177732 |
| <1e−07 | <1e−07 | 62.73 | 31.33 | 2 | IGF2BP2 | 10644 | NM_001007225 | Hs.35354 | ENSG00000073792 |
| <1e−07 | <1e−07 | 42.82 | 22.9 | 1.87 | PDK1 | 5163 | NM_001278549 | Hs.470633 | ENSG00000152256 |
| <1e−07 | <1e−07 | 4076.61 | 2435.17 | 1.67 | B2M | 567 | NM_004048 | Hs.534255 | ENSG00000166710 |
| <1e−07 | <1e−07 | 128.73 | 53.12 | 2.42 | NEXN | 91624 | NM_001172309 | Hs.612385 | ENSG00000162614 |
| <1e−07 | <1e−07 | 18.45 | 7.93 | 2.33 | RAB30 | 27314 | NM_001286059 | Hs.40758 | ENSG00000137502 |
| <1e−07 | <1e−07 | 22.02 | 8.41 | 2.62 | CLDN5 | 7122 | NM_001130861 | Hs.505337 | ENSG00000184113 |
| <1e−07 | <1e−07 | 267.18 | 405.45 | 0.66 | PREX1 | 57580 | NM_020820 | Hs.153310 | ENSG00000124126 |
| <1e−07 | <1e−07 | 15.92 | 8.04 | 1.98 | MLLT3 | 4300 | NM_001286691 | Hs.317248 | ENSG00000171843 |
| <1e−07 | <1e−07 | 10.64 | 5.37 | 1.98 | C15orf54 | 400360 | NM_001302797 | Hs.376109 | ENSG00000175746 |
| <1e−07 | 0.00000294 | 19.49 | 9.03 | 2.16 | FNBP1L | 54874 | NM_001024948 | Hs.134060 | ENSG00000137942 |
| <1e−07 | 0.00000294 | 81.38 | 53.79 | 1.51 | XPO7 | 23039 | NM_001100161 | Hs.172685 | ENSG00000130227 |
| <1e−07 | 0.00000294 | 35.66 | 14.6 | 2.44 | MT-TI | 4565 | | | |
| <1e−07 | 0.00000294 | 50.26 | 32.01 | 1.57 | MAP4K5 | 11183 | NM_006575 | Hs.130491 | ENSG00000012983 |
| <1e−07 | 0.00000294 | 24.32 | 10.51 | 2.31 | VWF | 7450 | NM_000552 | Hs.440848 | ENSG00000110799 |
| <1e−07 | 0.00000294 | 12.47 | 6.59 | 1.89 | BET1 | 10282 | NM_001317739 | Hs.489132 | ENSG00000105829 |
| <1e−07 | 0.00000294 | 13.52 | 23.21 | 0.58 | RXRB | 6257 | NM_001270401 | Hs.388034 | ENSG00000204231 |
| <1e−07 | 0.00000294 | 11.09 | 6 | 1.85 | ARMCX6 | 54470 | NM_001009584 | Hs.83530 | ENSG00000198960 |
| <1e−07 | 0.00000294 | 426.41 | 164.18 | 2.6 | ITGA2B | 3674 | NM_000419 | Hs.411312 | ENSG00000005961 |
| <1e−07 | 0.00000294 | 10.7 | 5.35 | 2 | ARMC3 | 219681 | NM_001282745 | Hs.659807 | ENSG00000165309 |
| <1e−07 | 0.00000294 | 12.74 | 6.68 | 1.91 | PLEKHA8P1 | 51054 | NM_015899 | Hs.233495 | ENSG00000134297 |
| <1e−07 | 0.00000294 | 13.52 | 6.79 | 1.99 | DNM1 | 1759 | NM_001005336 | Hs.522413 | ENSG00000106976 |
| <1e−07 | 0.00000294 | 725.31 | 468.22 | 1.55 | WIPF1 | 7456 | NM_001077269 | Hs.128067 | ENSG00000115935 |
| <1e−07 | 0.00000294 | 28.26 | 18.18 | 1.55 | MCU | 90550 | NM_001270679 | Hs.591366 | ENSG00000156026 |
| <1e−07 | 0.00000294 | 13.8 | 6.09 | 2.26 | ITGA6 | 3655 | NM_000210 | Hs.133397 | ENSG00000091409 |
| <1e−07 | 0.00000294 | 15.64 | 8.09 | 1.93 | CDC42BPA | 8476 | NM_003607 | Hs.35433 | ENSG00000143776 |
| <1e−07 | 0.00000294 | 13.31 | 6.26 | 2.13 | HGD | 3081 | NM_000187 | Hs.368254 | ENSG00000113924 |
| <1e−07 | 0.00000294 | 15.02 | 7.99 | 1.88 | MBNL1- | 401093 | NR_027037 | Hs.725347 | ENSG00000229619 |
| | | | | | AS1 | | | | |
| <1e−07 | 0.00000294 | 160.48 | 99.58 | 1.61 | SLC44A2 | 57153 | NM_001145056 | Hs.534560 | ENSG00000129353 |
| <1e−07 | 0.00000294 | 172.3 | 111.23 | 1.55 | RASA3 | 22821 | NM_001320821 | Hs.593075 | ENSG00000185989 |
| <1e−07 | 0.00000294 | 24.15 | 13.82 | 1.75 | SRSF8 | 10929 | NM_032102 | Hs.476680 | ENSG00000263465 |
| <1e−07 | 0.00000294 | 30.29 | 47.05 | 0.64 | PPMIM | 132160 | NM_001122870 | Hs.373560 | ENSG00000164088 |
| <1e−07 | 0.00000294 | 558.64 | 864.73 | 0.65 | AKAP13 | 11214 | NM_001270546 | Hs.459211 | ENSG00000170776 |
| <1e−07 | 0.00000294 | 106.96 | 179.79 | 0.59 | HK3 | 3101 | NM_002115 | Hs.411695 | ENSG00000160883 |
| <1e−07 | 0.00000294 | 96.07 | 49.18 | 1.95 | MBNL3 | 55796 | NM_001170701 | Hs.105134 | ENSG00000076770 |
| <1e−07 | 0.00000294 | 10.77 | 5.47 | 1.97 | AC007262.2 | | | | |
| 0.0000001 | 0.00000294 | 8.74 | 5.58 | 1.56 | PTMAP4 | 5761 | | | |
| 0.0000001 | 0.00000294 | 203.63 | 310.44 | 0.66 | PLCB2 | 5330 | NM_001284297 | Hs.355888 | ENSG00000137841 |
| 0.0000001 | 0.00000294 | 25.63 | 13.75 | 1.87 | H2BC15 | 8341 | | | |
| 0.0000001 | 0.00000294 | 237.22 | 148.57 | 1.6 | GSE1 | 23199 | NM_001134473 | Hs.461647 | ENSG00000131149 |
| 0.0000001 | 0.00000294 | 15.8 | 8.7 | 1.82 | TXNL4B | 54957 | NM_001142317 | Hs.134406 | ENSG00000140830 |
| 0.0000001 | 0.00000294 | 293.13 | 167.11 | 1.75 | CYB5R3 | 1727 | NM_000398 | Hs.561064 | ENSG00000100243 |
| 0.0000001 | 0.00000294 | 12.07 | 5.79 | 2.08 | ITGA2 | 3673 | NM_002203 | Hs.482077 | ENSG00000164171 |
| 0.0000001 | 0.00000294 | 14.33 | 6.66 | 2.15 | SERPINE1 | 5054 | NM_000602 | Hs.414795 | ENSG00000106366 |
| 0.0000001 | 0.00000294 | 10.32 | 5.23 | 1.97 | DNAJC6 | 9829 | NM_001256864 | Hs.647643 | ENSG00000116675 |
| 0.0000001 | 0.00000294 | 295.82 | 469.81 | 0.63 | APOBR | 55911 | NM_018690 | Hs.200333 | ENSG00000184730 |
| 0.0000001 | 0.00000294 | 47.52 | 18.7 | 2.54 | MT-TS1 | 4574 | | | |
| 0.0000001 | 0.00000294 | 173.03 | 93.8 | 1.84 | H2BC12 | 85236 | | | |
| 0.0000001 | 0.00000294 | 102.21 | 24.68 | 4.14 | PF4V1 | 5197 | NM_002620 | Hs.72933 | ENSG00000109272 |
| 0.0000001 | 0.00000294 | 23.99 | 12.3 | 1.95 | ORMDL3 | 94103 | NM_001320801 | Hs.514151 | ENSG00000172057 |
| 0.0000001 | 0.00000294 | 12.15 | 6.4 | 1.9 | SUSD3 | 203328 | NM_001287005 | Hs.88417 | ENSG00000157303 |
| 0.0000001 | 0.00000294 | 9.27 | 5.26 | 1.76 | SHROOM4 | 57477 | NM_020717 | Hs.420541 | ENSG00000158352 |
| 0.0000001 | 0.00000294 | 10.57 | 5.54 | 1.91 | AP001189.3 | | | | |
| 0.0000002 | 0.00000564 | 66.41 | 33.23 | 2 | H2BC5 | 3017 | | | |
| 0.0000002 | 0.00000564 | 41.65 | 23.58 | 1.77 | ARHGAP32 | 9743 | NM_001142685 | Hs.440379 | ENSG00000134909 |
| 0.0000002 | 0.00000564 | 24.15 | 11.64 | 2.07 | ACVR2A | 92 | NM_001278579 | Hs.470174 | ENSG00000121989 |
| 0.0000002 | 0.00000564 | 11.14 | 5.45 | 2.04 | F2RL3 | 9002 | NM_003950 | Hs.137574 | ENSG00000127533 |
| 0.0000002 | 0.00000564 | 15.21 | 6.91 | 2.2 | MYCT1 | 80177 | NM_025107 | Hs.18160 | ENSG00000120279 |
| 0.0000002 | 0.00000564 | 14.92 | 7.55 | 1.98 | ANGPT1 | 284 | NM_001146 | Hs.369675 | ENSG00000154188 |
| 0.0000002 | 0.00000564 | 115.42 | 71.12 | 1.62 | PSTPIP2 | 9050 | NM_024430 | Hs.567384 | ENSG00000152229 |
| 0.0000002 | 0.00000564 | 29.31 | 9.56 | 3.07 | PDZK1IP1 | 10158 | NM_005764 | Hs.431099 | ENSG00000162366 |
| 0.0000002 | 0.00000564 | 156.75 | 96.73 | 1.62 | ILK | 3611 | NM_001014794 | Hs.706355 | ENSG00000166333 |
| 0.0000002 | 0.00000564 | 137.93 | 211.32 | 0.65 | ADCY7 | 113 | NM_001114 | Hs.513578 | ENSG00000121281 |
| 0.0000002 | 0.00000564 | 91.37 | 51.13 | 1.79 | PARVB | 29780 | NM_001003828 | Hs.475074 | ENSG00000188677 |
| 0.0000002 | 0.00000564 | 11.41 | 5.67 | 2.01 | MLC1 | 23209 | NM_015166 | Hs.517729 | ENSG00000100427 |
| 0.0000002 | 0.00000564 | 336.14 | 519.2 | 0.65 | EHBP1L1 | 254102 | NM_001099409 | Hs.502867 | ENSG00000173442 |
| 0.0000002 | 0.00000564 | 13.33 | 6.65 | 2 | CRACD | 57482 | | | |
| 0.0000002 | 0.00000564 | 16.37 | 7.54 | 2.17 | AC127502.2 | | | | |
| 0.0000002 | 0.00000564 | 29.01 | 45.32 | 0.64 | LTB4R | 1241 | NM_001143919 | Hs.567248 | ENSG00000213903 |
| 0.0000002 | 0.00000564 | 15.27 | 8.54 | 1.79 | BARD1 | 580 | NM_000465 | Hs.591642 | ENSG00000138376 |
| 0.0000002 | 0.00000564 | 13.18 | 7.04 | 1.87 | CLCN4 | 1183 | NM_001256944 | Hs.495674 | ENSG00000073464 |
| 0.0000002 | 0.00000564 | 11.93 | 5.64 | 2.12 | GRHL1 | 29841 | NM_198182 | Hs.418493 | ENSG00000134317 |
| 0.0000002 | 0.00000564 | 120.65 | 183.47 | 0.66 | FCGRT | 2217 | NM_001136019 | Hs.111903 | ENSG00000104870 |
| 0.0000003 | 0.00000816 | 16.43 | 6.97 | 2.36 | GP5 | 2814 | NM_004488 | Hs.73734 | ENSG00000178732 |
| 0.0000003 | 0.00000816 | 23.52 | 12.34 | 1.91 | LGALS12 | 85329 | NM_001142535 | Hs.502774 | ENSG00000133317 |
| 0.0000003 | 0.00000816 | 16.25 | 8.25 | 1.97 | MBOAT2 | 129642 | NM_001321265 | Hs.467634 | ENSG00000143797 |
| 0.0000003 | 0.00000816 | 25.47 | 15.72 | 1.62 | DSTN | 11034 | NM_001011546 | Hs.304192 | ENSG00000125868 |
| 0.0000003 | 0.00000816 | 16.01 | 8.65 | 1.85 | DYNLRB1 | 83658 | NM_001281727 | Hs.593920 | ENSG00000125971 |
| 0.0000003 | 0.00000816 | 31.26 | 18.95 | 1.65 | UBE2E3 | 10477 | NM_001278554 | Hs.470804 | ENSG00000170035 |
| 0.0000003 | 0.00000816 | 26.29 | 9.43 | 2.79 | AC123912.4 | | | | |
| 0.0000003 | 0.00000816 | 11.04 | 5.71 | 1.93 | AC090409.1 | | | | |
| 0.0000003 | 0.00000816 | 8.01 | 5.03 | 1.59 | AC078883.1 | | | | |
| 0.0000003 | 0.00000816 | 485.9 | 322.98 | 1.5 | PTPRJ | 5795 | NM_001098503 | Hs.318547 | ENSG00000149177 |
| 0.0000003 | 0.00000816 | 50.01 | 30.42 | 1.64 | WDR44 | 54521 | NM_001184965 | Hs.98510 | ENSG00000131725 |
| 0.0000003 | 0.00000816 | 33.94 | 21.72 | 1.56 | ACP1 | 52 | NM_001040649 | Hs.558296 | ENSG00000143727 |
| 0.0000003 | 0.00000816 | 9.67 | 5.6 | 1.73 | SLC22A23 | 63027 | NM_001286455 | Hs.713588 | ENSG00000137266 |
| 0.0000003 | 0.00000816 | 35.96 | 15.79 | 2.28 | GNB5 | 10681 | NM_006578 | Hs.155090 | ENSG00000069966 |
| 0.0000003 | 0.00000816 | 96.81 | 150.71 | 0.64 | MAST3 | 23031 | NM_015016 | Hs.466184 | ENSG00000099308 |
| 0.0000003 | 0.00000816 | 339.03 | 528.99 | 0.64 | ARAP1 | 116985 | NM_001040118 | Hs.503165 | ENSG00000186635 |
| 0.0000003 | 0.00000816 | 1097.93 | 706 | 1.56 | TPM4 | 7171 | NM_001145160 | Hs.631618 | ENSG00000167460 |
| 0.0000004 | 0.0000105 | 8.3 | 5.12 | 1.62 | STXBP5-AS1 | 729178 | NR_034115 | Hs.557608 | |
| 0.0000004 | 0.0000105 | 294.77 | 163.62 | 1.8 | ADIPOR1 | 51094 | NM_001290553 | Hs.5298 | ENSG00000159346 |
| 0.0000004 | 0.0000105 | 10.33 | 5.26 | 1.96 | AC113404.3 | | | | |
| 0.0000004 | 0.0000105 | 10.16 | 5.36 | 1.9 | SCN1B | 6324 | NM_001037 | Hs.436646 | ENSG00000105711 |
| 0.0000004 | 0.0000105 | 28.14 | 48.88 | 0.58 | TRPM2 | 7226 | NM_001001188 | Hs.369759 | ENSG00000142185 |
| 0.0000004 | 0.0000105 | 30.88 | 8.68 | 3.56 | OSBP2 | 23762 | NM_001003812 | Hs.517546 | ENSG00000184792 |
| 0.0000004 | 0.0000105 | 13.29 | 6.36 | 2.09 | PRKG1 | 5592 | NM_001098512 | Hs.407535 | ENSG00000185532 |
| 0.0000004 | 0.0000105 | 56.31 | 37.45 | 1.5 | VAMP7 | 6845 | NM_001145149 | Hs.24167 | ENSG00000124333 |
| 0.0000004 | 0.0000105 | 216.98 | 344.71 | 0.63 | TNRC18 | 84629 | NM_001013722 | Hs.520638 | ENSG00000182095 |
| 0.0000004 | 0.0000105 | 288.24 | 446.25 | 0.65 | CD93 | 22918 | NM_012072 | Hs.97199 | ENSG00000125810 |
| 0.0000004 | 0.0000105 | 23.84 | 37.29 | 0.64 | CES2 | 8824 | NM_003869 | Hs.282975 | ENSG00000172831 |
| 0.0000004 | 0.0000105 | 27.43 | 41.56 | 0.66 | ZNF646 | 9726 | NM_014699 | Hs.119273 | ENSG00000167395 |
| 0.0000004 | 0.0000105 | 8.77 | 5.01 | 1.75 | SLC18A2 | 6571 | NM_003054 | Hs.596992 | ENSG00000165646 |
| 0.0000005 | 0.0000128 | 41.69 | 24.68 | 1.69 | AC135983.3 | | | | |
| 0.0000005 | 0.0000128 | 8.84 | 5.13 | 1.72 | LYPLAL1-DT | 643723 | NR_038845 | Hs.519774 | |
| 0.0000005 | 0.0000128 | 10.79 | 6.03 | 1.79 | TESPA1 | 9840 | NM_001098815 | Hs.33187 | ENSG00000135426 |
| 0.0000005 | 0.0000128 | 788.51 | 461.78 | 1.71 | NCOA4 | 8031 | NM_001145260 | Hs.643658 | ENSG00000266412 |
| 0.0000005 | 0.0000128 | 90.73 | 139.45 | 0.65 | SLC38A10 | 124565 | NM_001037984 | Hs.352240 | ENSG00000157637 |
| 0.0000005 | 0.0000128 | 92.88 | 50.03 | 1.86 | INF2 | 64423 | NM_001031714 | Hs.24956 | ENSG00000203485 |
| 0.0000005 | 0.0000128 | 18.36 | 11.51 | 1.6 | NTAN1 | 123803 | NM_001270766 | Hs.592045 | ENSG00000157045 |
| 0.0000005 | 0.0000128 | 9.76 | 5.33 | 1.83 | LIPH | 200879 | NM_139248 | Hs.68864 | ENSG00000163898 |
| 0.0000005 | 0.0000128 | 22.26 | 11.69 | 1.9 | CYTOR | 112597 | NR_024204 | Hs.652166 | ENSG00000222041 |
| 0.0000005 | 0.0000128 | 23.72 | 9.99 | 2.37 | IRS1 | 3667 | NM_005544 | Hs.471508 | ENSG00000169047 |
| 0.0000005 | 0.0000128 | 169.89 | 110.13 | 1.54 | TUBA1B | 10376 | NM_006082 | Hs.524390 | ENSG00000123416 |
| 0.0000005 | 0.0000128 | 13.23 | 6.64 | 1.99 | MCM6 | 4175 | NM_005915 | Hs.444118 | ENSG00000076003 |
| 0.0000005 | 0.0000128 | 28.7 | 43.21 | 0.66 | RGL2 | 5863 | NM_001243738 | Hs.509622 | ENSG00000237441 |
| 0.0000006 | 0.0000152 | 7.09 | 10.98 | 0.65 | COQ8B | 79934 | NM_001142555 | Hs.130712 | ENSG00000123815 |
| 0.0000006 | 0.0000152 | 9.32 | 5.12 | 1.82 | GATA1 | 2623 | NM_002049 | Hs.765 | ENSG00000102145 |
| 0.0000006 | 0.0000152 | 603.95 | 376.5 | 1.6 | GPX1 | 2876 | NM_000581 | Hs.76686 | ENSG00000233276 |
| 0.0000007 | 0.0000174 | 8.67 | 5.22 | 1.66 | PCED1B | 91523 | NM_001281429 | Hs.560100 | ENSG00000179715 |
| 0.0000007 | 0.0000174 | 14.52 | 24.32 | 0.6 | TXNRD2 | 10587 | NM_001282512 | Hs.443430 | ENSG00000184470 |
| 0.0000007 | 0.0000174 | 9.46 | 5.27 | 1.8 | PTPRF | 5792 | NM_001329137 | Hs.272062 | ENSG00000142949 |
| 0.0000007 | 0.0000174 | 94.01 | 61.87 | 1.52 | UQCRH | 7388 | NM_001297565 | Hs.481571 | ENSG00000173660 |
| 0.0000007 | 0.0000174 | 12.63 | 21.17 | 0.6 | EIF2B4 | 8890 | NM_001034116 | Hs.169474 | ENSG00000115211 |
| 0.0000007 | 0.0000174 | 8.83 | 5.21 | 1.69 | AP002478.1 | | | | |
| 0.0000008 | 0.0000195 | 34.05 | 8.95 | 3.8 | RPL23AP7 | 118433 | NR_000029 | Hs.595056 | |
| 0.0000008 | 0.0000195 | 410.58 | 648.6 | 0.63 | IL17RA | 23765 | NM_001289905 | Hs.48353 | ENSG00000177663 |
| 0.0000008 | 0.0000195 | 8.95 | 5.07 | 1.77 | PTP4A2P2 | 5769 | | Hs.661913 | |
| 0.0000008 | 0.0000195 | 8.84 | 5.3 | 1.67 | SMIM27 | 100129250 | NM_001349118 | Hs.664395 | ENSG00000235453 |
| 0.0000008 | 0.0000195 | 102.02 | 156.47 | 0.65 | SIPA1 | 6494 | NM_006747 | Hs.530477 | ENSG00000213445 |
| 0.0000009 | 0.0000217 | 29.5 | 17.29 | 1.71 | VKORC1L1 | 154807 | NM_001284342 | Hs.427232 | ENSG00000196715 |
| 0.0000009 | 0.0000217 | 28.95 | 16.96 | 1.71 | PANX1 | 24145 | NM_015368 | Hs.591976 | ENSG00000110218 |
| 0.000001 | 0.0000239 | 9.16 | 5.39 | 1.7 | ST8SIA6 | 338596 | NM_001004470 | Hs.677766 | ENSG00000148488 |
| 0.000001 | 0.0000239 | 11.42 | 6.16 | 1.85 | DENND2C | 163259 | NM_001256404 | Hs.654928 | ENSG00000175984 |
| 0.000001 | 0.0000239 | 42.52 | 19.81 | 2.15 | AFAP1 | 60312 | NM_001134647 | Hs.529369 | ENSG00000196526 |
| 0.000001 | 0.0000239 | 9.06 | 5.28 | 1.72 | B4GALT6 | 9331 | NM_001330570 | Hs.591063 | ENSG00000118276 |
| 0.0000011 | 0.0000261 | 13.38 | 7.94 | 1.69 | TM2D2 | 83877 | NM_001024380 | Hs.7471 | ENSG00000169490 |
| 0.0000012 | 0.0000281 | 8.35 | 5.1 | 1.64 | PLCH1 | 23007 | NM_001130960 | Hs.567423 | ENSG00000114805 |
| 0.0000012 | 0.0000281 | 46.07 | 26.84 | 1.72 | CRAT | 1384 | NM_000755 | Hs.12068 | ENSG00000095321 |
| 0.0000012 | 0.0000281 | 7.74 | 11.9 | 0.65 | GALK1 | 2584 | NM_000154 | Hs.407966 | ENSG00000108479 |
| 0.0000012 | 0.0000281 | 13.25 | 5.98 | 2.22 | POLR3G | 10622 | NM_006467 | Hs.282387 | ENSG00000113356 |
| 0.0000012 | 0.0000281 | 174.32 | 286.3 | 0.61 | DYSF | 8291 | NM_001130455 | Hs.252180 | ENSG00000135636 |
| 0.0000012 | 0.0000281 | 12.81 | 6.7 | 1.91 | PROSER2 | 254427 | NM_153256 | Hs.435775 | ENSG00000148426 |
| 0.0000013 | 0.0000301 | 9.78 | 5.33 | 1.83 | KIF28P | 100130097 | | Hs.739045 | |
| 0.0000013 | 0.0000301 | 59.56 | 34.36 | 1.73 | ETFA | 2108 | NM_000126 | Hs.39925 | ENSG00000140374 |
| 0.0000013 | 0.0000301 | 30.2 | 53.49 | 0.56 | FRAT1 | 10023 | NM_005479 | Hs.126057 | ENSG00000165879 |
| 0.0000013 | 0.0000301 | 14.17 | 8.63 | 1.64 | MTRF1L | 54516 | NM_001114184 | Hs.225836 | ENSG00000112031 |
| 0.0000014 | 0.0000318 | 8.5 | 5.16 | 1.65 | AL157895.2 | | | | |
| 0.0000014 | 0.0000318 | 125.65 | 81.46 | 1.54 | HACD4 | 401494 | NM_001010915 | Hs.716678 | ENSG00000188921 |
| 0.0000014 | 0.0000318 | 47.87 | 75.15 | 0.64 | OSCAR | 126014 | NM_001282349 | Hs.347655 | ENSG00000170909 |
| 0.0000014 | 0.0000318 | 111.32 | 167.72 | 0.66 | RAB3D | 9545 | NM_004283 | Hs.744916 | ENSG00000105514 |
| 0.0000014 | 0.0000318 | 10.31 | 5.36 | 1.92 | LINC02757 | 101928813 | | | |
| 0.0000014 | 0.0000318 | 41.07 | 65.42 | 0.63 | TBC1D2 | 55357 | NM_001267571 | Hs.371016 | ENSG00000095383 |
| 0.0000014 | 0.0000318 | 11.4 | 5.96 | 1.91 | PGM2L1 | 283209 | NM_173582 | Hs.26612 | ENSG00000165434 |
| 0.0000015 | 0.000034 | 53.93 | 34.1 | 1.58 | MAP4K2 | 5871 | NM_001307990 | Hs.534341 | ENSG00000168067 |
| 0.0000015 | 0.000034 | 91.52 | 54 | 1.69 | CDKN2D | 1032 | NM_001800 | Hs.435051 | ENSG00000129355 |
| 0.0000016 | 0.0000361 | 28.06 | 10.71 | 2.62 | FSTL1 | 11167 | NM_007085 | Hs.269512 | ENSG00000163430 |
| 0.0000016 | 0.0000361 | 6.15 | 10.55 | 0.58 | MAP3K6 | 9064 | NM_001297609 | Hs. 194694 | ENSG00000142733 |
| 0.0000018 | 0.0000403 | 18.58 | 8.73 | 2.13 | ANKRD9 | 122416 | NM_001348651 | Hs.432945 | ENSG00000156381 |
| 0.0000018 | 0.0000403 | 6.83 | 11.57 | 0.59 | AC108134.3 | | | | |
| 0.0000018 | 0.0000403 | 128.39 | 69.83 | 1.84 | H3P6 | 440926 | | | |
| 0.0000019 | 0.0000423 | 17.42 | 10 | 1.74 | DCAKD | 79877 | NM_001128631 | Hs.463148 | ENSG00000172992 |
| 0.0000019 | 0.0000423 | 20.46 | 10.66 | 1.92 | SEPTIN1 | 1731 | | | |
| 0.0000021 | 0.0000464 | 8.05 | 5.26 | 1.53 | AC104083.1 | | | | |
| 0.0000022 | 0.0000482 | 376.51 | 566.25 | 0.66 | VSIR | 64115 | NM_022153 | Hs.47382 | ENSG00000107738 |
| 0.0000022 | 0.0000482 | 23.1 | 38.37 | 0.6 | OSBPL5 | 114879 | NM_001144063 | Hs.436166 | ENSG00000021762 |
| 0.0000022 | 0.0000482 | 27.89 | 14.28 | 1.95 | FTH1P20 | 729009 | | | |
| 0.0000026 | 0.0000565 | 11.36 | 6.65 | 1.71 | ITGB3BP | 23421 | NM_001206739 | Hs.166539 | ENSG00000142856 |
| 0.0000026 | 0.0000565 | 93.38 | 37.52 | 2.49 | MT-TA | 4553 | | | |
| 0.0000026 | 0.0000565 | 22.19 | 34.52 | 0.64 | CEP164 | 22897 | NM_001271933 | Hs.504009 | ENSG00000110274 |
| 0.0000027 | 0.0000586 | 38.12 | 24.99 | 1.53 | UBASH3B | 84959 | NM_032873 | Hs.444075 | ENSG00000154127 |
| 0.0000028 | 0.0000605 | 24.06 | 11.42 | 2.11 | DBN1 | 1627 | NM_004395 | Hs.130316 | ENSG00000113758 |
| 0.0000029 | 0.0000624 | 63.03 | 34.15 | 1.85 | DYNLL1 | 8655 | NM_001037494 | Hs.5120 | ENSG00000088986 |
| 0.0000029 | 0.0000624 | 8.08 | 12.52 | 0.65 | KCTD17 | 79734 | NM_001282684 | Hs.517597 | ENSG00000100379 |
| 0.0000029 | 0.0000624 | 112.5 | 168.76 | 0.67 | KIAA0930 | 23313 | NM_001009880 | Hs.592207 | ENSG00000100364 |
| 0.000003 | 0.0000643 | 18.35 | 30.17 | 0.61 | IKBKE | 9641 | NM_001193321 | Hs.321045 | ENSG00000263528 |
| 0.0000031 | 0.0000661 | 16.92 | 9.59 | 1.76 | FUT8 | 2530 | NM_004480 | Hs.597649 | ENSG00000033170 |
| 0.0000032 | 0.0000681 | 28.47 | 43.94 | 0.65 | GRAMD4 | 23151 | NM_015124 | Hs.475150 | ENSG00000075240 |
| 0.0000032 | 0.0000681 | 25.72 | 41.97 | 0.61 | DEF8 | 54849 | NM_001242816 | Hs.62771 | ENSG00000140995 |
| 0.0000033 | 0.0000701 | 19.54 | 9.69 | 2.02 | P2RY1 | 5028 | NM_002563 | Hs.79881 | ENSG00000169860 |
| 0.0000035 | 0.0000739 | 12.25 | 7.76 | 1.58 | CRYL1 | 51084 | NM_015974 | Hs.370703 | ENSG00000165475 |
| 0.0000036 | 0.0000758 | 61.2 | 92.59 | 0.66 | SLC12A9 | 56996 | NM_001267812 | Hs.521087 | ENSG00000146828 |
| 0.0000039 | 0.0000812 | 72.1 | 41.82 | 1.72 | SRC | 6714 | NM_005417 | Hs.195659 | ENSG00000197122 |
| 0.0000039 | 0.0000812 | 22.58 | 35.89 | 0.63 | POR | 5447 | NM_000941 | Hs.354056 | ENSG00000127948 |
| 0.0000039 | 0.0000812 | 7.62 | 5.06 | 1.51 | THEM5 | 284486 | NM_182578 | Hs.132648 | ENSG00000196407 |
| 0.0000039 | 0.0000812 | 38.05 | 19.89 | 1.91 | ABHD4 | 63874 | NM_022060 | Hs.445665 | ENSG00000100439 |
| 0.0000042 | 0.0000868 | 18.15 | 9.21 | 1.97 | EFCAB13 | 124989 | NM_001195192 | Hs.463303 | ENSG00000178852 |
| 0.0000043 | 0.0000883 | 9.4 | 14.22 | 0.66 | ARHGEF18 | 23370 | NM_001130955 | Hs.465761 | ENSG00000104880 |
| 0.0000043 | 0.0000883 | 11.34 | 6.91 | 1.64 | DNAJC9 | 23234 | NM_015190 | Hs.408577 | ENSG00000213551 |
| 0.0000043 | 0.0000883 | 58.98 | 93.16 | 0.63 | KDM4B | 23030 | NM_015015 | Hs.654816 | ENSG00000127663 |
| 0.0000044 | 0.00009 | 22.72 | 9.58 | 2.37 | MT-TQ | 4572 | | | |
| 0.0000044 | 0.00009 | 1222.41 | 1965.18 | 0.62 | DUSP1 | 1843 | NM_004417 | Hs.171695 | ENSG00000120129 |
| 0.0000046 | 0.000094 | 77.34 | 28.64 | 2.7 | TENT5C | 54855 | | | |
| 0.0000047 | 0.0000955 | 96.56 | 41.82 | 2.31 | TNS1 | 7145 | NM_001308022 | Hs.471381 | ENSG00000079308 |
| 0.0000047 | 0.0000955 | 9.95 | 5.52 | 1.8 | DDX11L2 | 84771 | NR_024004 | Hs.712940 | |
| 0.0000048 | 0.000097 | 26.67 | 16.09 | 1.66 | ICAM2 | 3384 | NM_000873 | Hs.431460 | ENSG00000108622 |
| 0.0000048 | 0.000097 | 21.98 | 10.15 | 2.17 | KIFC3 | 3801 | NM_001130099 | Hs.23131 | ENSG00000140859 |
| 0.0000048 | 0.000097 | 41.48 | 16.46 | 2.52 | HSPB1 | 3315 | NM_001540 | Hs.520973 | ENSG00000106211 |
| 0.000005 | 0.000101 | 146.46 | 249.25 | 0.59 | STAB1 | 23166 | NM_015136 | Hs.301989 | ENSG00000010327 |
| 0.0000052 | 0.000104 | 17.43 | 10.18 | 1.71 | SLC39A3 | 29985 | NM_144564 | Hs.515046 | ENSG00000141873 |
| 0.0000053 | 0.000106 | 126.3 | 201.98 | 0.63 | MEFV | 4210 | NM_000243 | Hs.632221 | ENSG00000103313 |
| 0.0000056 | 0.000111 | 48.1 | 30.24 | 1.59 | NCOA7 | 135112 | NM_001122842 | Hs.171426 | ENSG00000111912 |
| 0.0000056 | 0.000111 | 279.98 | 180.56 | 1.55 | TIMP1 | 7076 | NM_003254 | Hs.522632 | ENSG00000102265 |
| 0.0000056 | 0.000111 | 38.58 | 17.04 | 2.26 | GATA2 | 2624 | NM_001145661 | Hs.367725 | ENSG00000179348 |
| 0.0000058 | 0.000114 | 15.53 | 26.03 | 0.6 | SLC22A15 | 55356 | NM_018420 | Hs.125482 | ENSG00000163393 |
| 0.0000059 | 0.000116 | 51.23 | 78.36 | 0.65 | DEF6 | 50619 | NM_022047 | Hs.15476 | ENSG00000023892 |
| 0.0000059 | 0.000116 | 29.4 | 51.35 | 0.57 | FRAT2 | 23401 | NM_012083 | Hs.140720 | ENSG00000181274 |
| 0.0000062 | 0.000121 | 8.53 | 5.25 | 1.62 | TMEM106C | 79022 | NM_001143841 | Hs.596726 | ENSG00000134291 |
| 0.0000063 | 0.000123 | 205.43 | 135.73 | 1.51 | MKRN1 | 23608 | NM_001145125 | Hs.744883 | ENSG00000133606 |
| 0.0000065 | 0.000127 | 10.89 | 19.6 | 0.56 | FBXL19 | 54620 | NM_001099784 | Hs.152149 | ENSG00000099364 |
| 0.0000066 | 0.000129 | 46.94 | 27.38 | 1.71 | INSIG1 | 3638 | NM_001346590 | Hs.520819 | ENSG00000186480 |
| 0.0000067 | 0.00013 | 10.65 | 6.79 | 1.57 | ZFYVE21 | 79038 | NM_001198953 | Hs.592322 | ENSG00000100711 |
| 0.0000067 | 0.00013 | 10.99 | 5.81 | 1.89 | KCND3 | 3752 | NM_004980 | Hs.666367 | ENSG00000171385 |
| 0.0000068 | 0.000132 | 38.79 | 58.3 | 0.67 | SIGLEC9 | 27180 | NM_001198558 | Hs.245828 | ENSG00000129450 |
| 0.0000071 | 0.000137 | 8.9 | 5.26 | 1.69 | AC137932.2 | | | | |
| 0.0000072 | 0.000139 | 18.22 | 11.69 | 1.56 | TUBGCP4 | 27229 | NM_001286414 | Hs.584887 | ENSG00000137822 |
| 0.0000074 | 0.000143 | 86.57 | 44.88 | 1.93 | GAS2L1 | 10634 | NM_001278730 | Hs.322852 | ENSG00000185340 |
| 0.0000075 | 0.000144 | 25.77 | 16.05 | 1.61 | REPS2 | 9185 | NM_001080975 | Hs.186810 | ENSG00000169891 |
| 0.0000075 | 0.000144 | 8.35 | 5.13 | 1.63 | ACOT7 | 11332 | NM_007274 | Hs.126137 | ENSG00000097021 |
| 0.0000078 | 0.000149 | 7.78 | 5.09 | 1.53 | EHD2 | 30846 | NM_014601 | Hs. 744963 | ENSG00000024422 |
| 0.0000079 | 0.00015 | 9.52 | 5.41 | 1.76 | CTNNAL1 | 8727 | NM_001286974 | Hs.58488 | ENSG00000119326 |
| 0.0000079 | 0.00015 | 77.99 | 122.03 | 0.64 | RHBDF2 | 79651 | NM_001005498 | Hs.464157 | ENSG00000129667 |
| 0.000008 | 0.000152 | 165.67 | 253.15 | 0.65 | RXRA | 6256 | NM_001291920 | Hs.590886 | ENSG00000186350 |
| 0.0000084 | 0.000159 | 825.68 | 1383.41 | 0.6 | PLEC | 5339 | NM_000445 | Hs.434248 | ENSG00000178209 |
| 0.0000086 | 0.000162 | 12.34 | 20.58 | 0.6 | CHMP7 | 91782 | NM_001317899 | Hs.5019 | ENSG00000147457 |
| 0.0000088 | 0.000165 | 22.31 | 13.65 | 1.63 | IFI27L2 | 83982 | NM_032036 | Hs.94695 | ENSG00000119632 |
| 0.0000089 | 0.000167 | 550.11 | 827.56 | 0.66 | GRN | 2896 | NM_001012479 | Hs.514220 | ENSG00000030582 |
| 0.0000092 | 0.000172 | 46.32 | 29.17 | 1.59 | H2AJ | 55766 | | | |
| 0.0000094 | 0.000175 | 15.69 | 9.54 | 1.65 | MTHFD2L | 441024 | NM_001004346 | Hs.721011 | ENSG00000163738 |
| 0.0000104 | 0.000192 | 7.73 | 5.01 | 1.54 | SUCNR1 | 56670 | NM_033050 | Hs.133181 | ENSG00000198829 |
| 0.0000108 | 0.000198 | 3847.18 | 5923.11 | 0.65 | VCAN | 1462 | NM_001126336 | Hs.643801 | ENSG00000038427 |
| 0.0000116 | 0.000211 | 115.98 | 178.95 | 0.65 | SNX27 | 81609 | NM_001330723 | Hs.192326 | ENSG00000143376 |
| 0.0000118 | 0.000215 | 14.24 | 21.62 | 0.66 | TBC1D10C | 374403 | NM_001256508 | Hs.534648 | ENSG00000175463 |
| 0.0000119 | 0.000216 | 18.47 | 30.89 | 0.6 | BAHCC1 | 57597 | NM_001080519 | Hs.514580 | ENSG00000266074 |
| 0.000013 | 0.000233 | 66.56 | 42.37 | 1.57 | TPST2 | 8459 | NM_001008566 | Hs.632768 | ENSG00000128294 |
| 0.0000141 | 0.000252 | 9.47 | 5.58 | 1.7 | KALRN | 8997 | NM_001024660 | Hs.8004 | ENSG00000160145 |
| 0.0000143 | 0.000255 | 40.24 | 61.29 | 0.66 | WDR81 | 124997 | NM_001163673 | Hs.234572 | ENSG00000167716 |
| 0.0000146 | 0.00026 | 8.01 | 5.09 | 1.57 | MYEOV | 26579 | NM_001293291 | Hs.523848 | ENSG00000172927 |
| 0.0000151 | 0.000267 | 7.93 | 5.23 | 1.52 | AC144652.1 | | | | |
| 0.0000155 | 0.000273 | 13.75 | 21.23 | 0.65 | TAB1 | 10454 | NM_006116 | Hs.507681 | ENSG00000100324 |
| 0.0000155 | 0.000273 | 9.01 | 5.58 | 1.62 | AL359644.1 | | | | |
| 0.0000165 | 0.000288 | 13.24 | 20.35 | 0.65 | CCDC22 | 28952 | NM_014008 | Hs.26333 | ENSG00000101997 |
| 0.0000165 | 0.000288 | 56.13 | 84.36 | 0.67 | GLT1D1 | 144423 | NM_144669 | Hs.655668 | ENSG00000151948 |
| 0.0000172 | 0.000299 | 29.96 | 17.81 | 1.68 | MYO1C | 4641 | NM_001080779 | Hs.286226 | ENSG00000197879 |
| 0.0000173 | 0.0003 | 50.58 | 32.45 | 1.56 | MFSD6 | 54842 | NM_017694 | Hs.418581 | ENSG00000151690 |
| 0.0000174 | 0.000301 | 109.89 | 53.4 | 2.06 | MT-TT | 4576 | | | |
| 0.0000175 | 0.000302 | 9.78 | 6.37 | 1.53 | ZNF778 | 197320 | NM_001201407 | Hs.647385 | ENSG00000170100 |
| 0.0000176 | 0.000303 | 14.86 | 25.86 | 0.57 | FLYWCH1 | 84256 | NM_001308068 | Hs.655321 | ENSG00000059122 |
| 0.0000178 | 0.000306 | 8.33 | 5.32 | 1.56 | ITGA9-AS1 | 101928153 | NR_110531 | Hs.657314 | ENSG00000235257 |
| 0.0000187 | 0.000321 | 24.14 | 37.9 | 0.64 | ABL1 | 25 | NM_005157 | Hs.431048 | ENSG00000097007 |
| 0.0000193 | 0.000329 | 9.44 | 5.62 | 1.68 | TRIM40 | 135644 | NM_001286633 | Hs.509439 | ENSG00000204614 |
| 0.0000195 | 0.000332 | 9.51 | 5.69 | 1.67 | EGFL7 | 51162 | NM_016215 | Hs.91481 | ENSG00000172889 |
| 0.00002 | 0.000339 | 27.43 | 42.8 | 0.64 | CARD9 | 64170 | NM_052813 | Hs.694071 | ENSG00000187796 |
| 0.0000201 | 0.000341 | 44.23 | 23.01 | 1.92 | NPRL3 | 8131 | NM_001039476 | Hs.19699 | ENSG00000103148 |
| 0.0000202 | 0.000342 | 213.18 | 128.32 | 1.66 | EPB41 | 2035 | NM_001166005 | Hs.175437 | ENSG00000159023 |
| 0.0000203 | 0.000343 | 65.67 | 40.71 | 1.61 | GABARAPL2 | 11345 | NM_007285 | Hs.461379 | ENSG00000034713 |
| 0.0000204 | 0.000343 | 29.45 | 17.33 | 1.7 | RHOF | 54509 | NM_019034 | Hs.707579 | ENSG00000139725 |
| 0.0000204 | 0.000343 | 99.23 | 50.66 | 1.96 | RBM38 | 55544 | NM_001291780 | Hs.236361 | ENSG00000132819 |
| 0.0000204 | 0.000343 | 21.95 | 13.52 | 1.62 | AIDA | 64853 | NM_022831 | Hs.156625 | ENSG00000186063 |
| 0.0000206 | 0.000346 | 8.65 | 5.42 | 1.6 | C1orf116 | 79098 | NM_001083924 | Hs.32417 | ENSG00000182795 |
| 0.0000211 | 0.000353 | 7.76 | 5.11 | 1.52 | LIPC | 3990 | NM_000236 | Hs.654472 | ENSG00000166035 |
| 0.0000215 | 0.000359 | 39.18 | 62.14 | 0.63 | CCAR2 | 57805 | NM_021174 | Hs.744848 | ENSG00000158941 |
| 0.0000217 | 0.000362 | 13.27 | 6.59 | 2.01 | ANKH | 56172 | NM_054027 | Hs.156727 | ENSG00000154122 |
| 0.0000218 | 0.000363 | 8.78 | 5.02 | 1.75 | BBOF1 | 80127 | NM_025057 | Hs.644621 | ENSG00000119636 |
| 0.0000218 | 0.000363 | 9.99 | 5.76 | 1.74 | NIPA1 | 123606 | NM_001142275 | Hs.511797 | ENSG00000170113 |
| 0.000022 | 0.000365 | 14.62 | 9.58 | 1.53 | CASP3 | 836 | NM_001354777 | Hs.141125 | ENSG00000164305 |
| 0.0000223 | 0.000369 | 38.77 | 6.66 | 5.82 | HBA1 | 3039 | NM_000558 | Hs.449630 | ENSG00000206172 |
| 0.0000231 | 0.00038 | 63.79 | 96.29 | 0.66 | HDAC4 | 9759 | NM_006037 | Hs.20516 | ENSG00000068024 |
| 0.0000231 | 0.00038 | 12.35 | 18.74 | 0.66 | AGPAT2 | 10555 | NM_001012727 | Hs.320151 | ENSG00000169692 |
| 0.0000237 | 0.000389 | 24.81 | 15.88 | 1.56 | BEX4 | 56271 | NM_001080425 | Hs.184736 | ENSG00000102409 |
| 0.0000238 | 0.000391 | 16.14 | 9.3 | 1.73 | EFNB1 | 1947 | NM_004429 | Hs.144700 | ENSG00000090776 |
| 0.0000242 | 0.000396 | 142.88 | 218.34 | 0.65 | ATG16L2 | 89849 | NM_001318766 | Hs.653186 | ENSG00000168010 |
| 0.0000245 | 0.0004 | 16.39 | 7.54 | 2.17 | MAP1B | 4131 | NM_001324255 | Hs.335079 | ENSG00000131711 |
| 0.0000247 | 0.000402 | 9.41 | 5.57 | 1.69 | OXTR | 5021 | NM_000916 | Hs.2820 | ENSG00000180914 |
| 0.0000256 | 0.000414 | 12.58 | 21.6 | 0.58 | MTARC1 | 64757 | | | |
| 0.0000266 | 0.000429 | 9.07 | 5.65 | 1.61 | ENO2 | 2026 | NM_001975 | Hs.511915 | ENSG00000111674 |
| 0.0000274 | 0.000439 | 126.44 | 208.39 | 0.61 | CCDC88B | 283234 | NM_032251 | Hs.98564 | ENSG00000168071 |
| 0.0000279 | 0.000445 | 31.25 | 52.43 | 0.6 | SCAF1 | 58506 | NM_021228 | Hs.103521 | ENSG00000126461 |
| 0.0000282 | 0.000449 | 9.22 | 5.47 | 1.69 | MT-TY | 4579 | | | |
| 0.0000283 | 0.000449 | 6.36 | 9.93 | 0.64 | ABHD6 | 57406 | NM_001320126 | Hs.476454 | ENSG00000163686 |
| 0.0000289 | 0.000457 | 8.06 | 5 | 1.61 | ARG2 | 384 | NM_001172 | Hs.226007 | ENSG00000081181 |
| 0.0000296 | 0.000468 | 9.47 | 14.24 | 0.67 | SPHK2 | 56848 | NM_001204158 | Hs.528006 | ENSG00000063176 |
| 0.0000299 | 0.000471 | 10.09 | 5.69 | 1.77 | PLXNB3 | 5365 | NM_001163257 | Hs.632833 | ENSG00000198753 |
| 0.0000307 | 0.000482 | 9.47 | 14.89 | 0.64 | FASTK | 10922 | NM_00125846] | Hs.647094 | ENSG00000164896 |
| 0.0000307 | 0.000482 | 11.2 | 7.17 | 1.56 | BICD1 | 636 | NM_001003398 | Hs.505202 | ENSG00000151746 |
| 0.000033 | 0.000517 | 15.51 | 24.88 | 0.62 | MOGS | 7841 | NM_001146158 | Hs.516119 | ENSG00000115275 |
| 0.0000333 | 0.00052 | 56.48 | 34.64 | 1.63 | PDE4D | 5144 | NM_001104631 | Hs.117545 | ENSG00000113448 |
| 0.0000344 | 0.000533 | 23.05 | 12.9 | 1.79 | PTGIR | 5739 | NM_000960 | Hs.458324 | ENSG00000160013 |
| 0.0000353 | 0.000546 | 19.33 | 32.03 | 0.6 | ARRDC1 | 92714 | NM_001317968 | Hs.12999 | ENSG00000197070 |
| 0.0000356 | 0.00055 | 13.72 | 8.45 | 1.62 | TTC39B | 158219 | NM_001168339 | Hs.563630 | ENSG00000155158 |
| 0.000037 | 0.000567 | 19.17 | 29.46 | 0.65 | DHX16 | 8449 | NM_001164239 | Hs.485060 | ENSG00000204560 |
| 0.0000378 | 0.000578 | 9.27 | 5.86 | 1.58 | CETN2 | 1069 | NM_004344 | Hs.82794 | ENSG00000147400 |
| 0.0000386 | 0.000588 | 63.06 | 96.02 | 0.66 | NLRP12 | 91662 | NM_001277126 | Hs.631573 | ENSG00000142405 |
| 0.000039 | 0.000593 | 29.63 | 47.08 | 0.63 | TECPR1 | 25851 | NM_015395 | Hs.592281 | ENSG00000205356 |
| 0.0000404 | 0.000612 | 11.25 | 17.76 | 0.63 | E4F1 | 1877 | NM_001288776 | Hs.513268 | ENSG00000167967 |
| 0.0000429 | 0.000646 | 51.31 | 33.67 | 1.52 | GPD2 | 2820 | NM_000408 | Hs.512382 | ENSG00000115159 |
| 0.000043 | 0.000647 | 10.03 | 5.92 | 1.69 | FN3K | 64122 | NM_022158 | Hs.151135 | ENSG00000167363 |
| 0.0000435 | 0.000654 | 18.36 | 11.81 | 1.55 | GLA | 2717 | NM_000169 | Hs.69089 | ENSG00000102393 |
| 0.0000446 | 0.000669 | 9.9 | 16.16 | 0.61 | DGKQ | 1609 | NM_001347 | Hs.584858 | ENSG00000145214 |
| 0.0000451 | 0.000676 | 19.79 | 30.63 | 0.65 | PLB1 | 151056 | NM_001170585 | Hs.444933 | ENSG00000163803 |
| 0.0000459 | 0.000686 | 11.06 | 17.01 | 0.65 | FBXO41 | 150726 | NM_001080410 | Hs.23158 | ENSG00000163013 |
| 0.0000461 | 0.000688 | 35.35 | 19.65 | 1.8 | KCNA3 | 3738 | NM_002232 | Hs.169948 | ENSG00000177272 |
| 0.0000461 | 0.000688 | 51.23 | 81.05 | 0.63 | ASGR2 | 433 | NM_001181 | Hs.654440 | ENSG00000161944 |
| 0.0000466 | 0.000694 | 15.15 | 23.64 | 0.64 | FARSA | 2193 | NM_004461 | Hs.23111 | ENSG00000179115 |
| 0.0000466 | 0.000694 | 105.6 | 162.89 | 0.65 | SBNO2 | 22904 | NM_001100122 | Hs.408708 | ENSG00000064932 |
| 0.0000492 | 0.000729 | 173.49 | 100.5 | 1.73 | ATP2A3 | 489 | NM_005173 | Hs.513870 | ENSG00000074370 |
| 0.0000505 | 0.000746 | 8.89 | 5.66 | 1.57 | PCDH9 | 5101 | NM_001318372 | Hs.654709 | ENSG00000184226 |
| 0.0000506 | 0.000747 | 8.87 | 5.28 | 1.68 | PPP1R14A | 94274 | NM_001243947 | Hs.631569 | ENSG00000167641 |
| 0.0000515 | 0.000759 | 17.79 | 31.62 | 0.56 | ADAP1 | 11033 | NM_001284308 | Hs.602573 | ENSG00000105963 |
| 0.0000546 | 0.0008 | 33.68 | 53.53 | 0.63 | SLC27A1 | 376497 | NM_198580 | Hs.363138 | ENSG00000130304 |
| 0.0000562 | 0.000822 | 26.17 | 40.48 | 0.65 | TMEM94 | 9772 | NM_001321148 | Hs.514474 | ENSG00000177728 |
| 0.0000562 | 0.000822 | 8.12 | 5.37 | 1.51 | LAPTM4B | 55353 | NM_018407 | Hs.492314 | ENSG00000104341 |
| 0.0000576 | 0.000837 | 36.05 | 105.25 | 0.34 | RNU4-1 | 26835 | NR_003925 | | ENSG00000200795 |
| 0.0000587 | 0.000851 | 11.02 | 5.81 | 1.9 | GCOM1 | 145781 | NM_001018090 | Hs.437256 | ENSG00000137878 |
| 0.000061 | 0.000881 | 26.73 | 40.35 | 0.66 | CCDC159 | 126075 | NM_001080503 | Hs.631636 | ENSG00000183401 |
| 0.000062 | 0.000892 | 12.53 | 7.38 | 1.7 | TSPAN2 | 10100 | NM_001308315 | Hs.310458 | ENSG00000134198 |
| 0.0000625 | 0.000898 | 131.09 | 199.07 | 0.66 | ARHGAP4 | 393 | NM_001164741 | Hs.701324 | ENSG00000089820 |
| 0.0000627 | 0.0009 | 45.95 | 29.19 | 1.57 | VIM-AS1 | 100507347 | NR_108060 | Hs.740502 | ENSG00000229124 |
| 0.0000628 | 0.0009 | 12.59 | 20.93 | 0.6 | ARL11 | 115761 | NM_138450 | Hs.558599 | ENSG00000152213 |
| 0.0000631 | 0.000903 | 11.75 | 18.2 | 0.65 | SNAPC4 | 6621 | NM_003086 | Hs.113265 | ENSG00000165684 |
| 0.0000649 | 0.000926 | 94.98 | 150.93 | 0.63 | CHST15 | 51363 | NM_001270764 | Hs.287537 | ENSG00000182022 |
| 0.0000661 | 0.000942 | 9.53 | 5.96 | 1.6 | CA13 | 377677 | NM_198584 | Hs.127189 | ENSG00000185015 |
| 0.0000691 | 0.000978 | 24.28 | 15.05 | 1.61 | MAGED2 | 10916 | NM_014599 | Hs.522665 | ENSG00000102316 |
| 0.0000705 | 0.000995 | 11.59 | 6.76 | 1.72 | CCDC9B | 388115 | NM_207380 | Hs.32433 | ENSG00000188549 |
| 0.0000718 | 0.00101 | 263.2 | 129.14 | 2.04 | MTND2P28 | 100652939 | | | |
| 0.0000728 | 0.00102 | 9.51 | 15.88 | 0.6 | MYO7A | 4647 | NM_000260 | Hs.370421 | ENSG00000137474 |
| 0.0000809 | 0.00112 | 24.08 | 38.64 | 0.62 | USP19 | 10869 | NM_001199160 | Hs.255596 | ENSG00000172046 |
| 0.0000818 | 0.00113 | 25.06 | 14.64 | 1.71 | GADD45A | 1647 | NM_001199741 | Hs.80409 | ENSG00000116717 |
| 0.0000882 | 0.00121 | 77.73 | 117.53 | 0.66 | ZNF467 | 168544 | NM_001329856 | Hs.726477 | ENSG00000181444 |
| 0.0000889 | 0.00121 | 27.39 | 47.57 | 0.58 | ABCA7 | 10347 | NM_019112 | Hs.134514 | ENSG00000064687 |
| 0.0000895 | 0.00122 | 73.22 | 115.09 | 0.64 | TNFRSF1A | 7132 | NM_001065 | Hs.279594 | ENSG00000067182 |
| 0.000091 | 0.00124 | 16871.1 | 26693.36 | 0.63 | MALAT1 | 378938 | NR_002819 | Hs.621695 | ENSG00000251562 |
| 0.0000957 | 0.0013 | 53.82 | 27.63 | 1.95 | MT-TW | 4578 | | | |
| 0.0000959 | 0.0013 | 25.94 | 41.15 | 0.63 | ACAP1 | 9744 | NM_014716 | Hs.337242 | ENSG00000072818 |
| 0.0001041 | 0.00139 | 21.5 | 35.3 | 0.61 | TOR4A | 54863 | NM_017723 | Hs.495541 | ENSG00000198113 |
| 0.0001058 | 0.00141 | 8.85 | 5.52 | 1.6 | AC147067.1 | | | | |
| 0.0001059 | 0.00141 | 10.63 | 6.8 | 1.56 | ZFPM1 | 161882 | NM_153813 | Hs.632218 | ENSG00000179588 |
| 0.0001084 | 0.00143 | 476.71 | 757.65 | 0.63 | SLC11A1 | 6556 | NM_000578 | Hs.591607 | ENSG00000018280 |
| 0.0001101 | 0.00145 | 13.05 | 7.86 | 1.66 | AC100810.1 | | | | |
| 0.0001111 | 0.00146 | 7.9 | 5.19 | 1.52 | AL022344.1 | | | | |
| 0.0001123 | 0.00147 | 19.21 | 28.82 | 0.67 | HAL | 3034 | NM_001258333 | Hs.190783 | ENSG00000084110 |
| 0.0001193 | 0.00155 | 20.96 | 31.74 | 0.66 | CEP250 | 11190 | NM_001035518 | Hs.443976 | ENSG00000126001 |
| 0.000123 | 0.00159 | 10.84 | 16.83 | 0.64 | MBD3 | 53615 | NM_001281453 | Hs.178728 | ENSG00000071655 |
| 0.0001321 | 0.00169 | 50.57 | 33.38 | 1.51 | FAM110A | 83541 | NM_001042353 | Hs.574822 | ENSG00000125898 |
| 0.0001327 | 0.00169 | 9.02 | 5.88 | 1.53 | SEPTIN4 | 5414 | | | |
| 0.0001401 | 0.00177 | 18.8 | 32.11 | 0.59 | FPR2 | 2358 | NM_001005738 | Hs.99855 | ENSG00000171049 |
| 0.000141 | 0.00178 | 21.06 | 31.65 | 0.67 | ZNF445 | 353274 | NM_181489 | Hs.250481 | ENSG00000185219 |
| 0.0001428 | 0.0018 | 7.9 | 5.11 | 1.55 | CAV2 | 858 | NM_001206747 | Hs.212332 | ENSG00000105971 |
| 0.0001484 | 0.00185 | 12.12 | 19.1 | 0.63 | TMEM250 | 90120 | NM_001256526 | Hs.287411 | ENSG00000238227 |
| 0.0001535 | 0.0019 | 74.04 | 117.26 | 0.63 | RASGRP4 | 115727 | NM_001146202 | Hs.130434 | ENSG00000171777 |
| 0.0001542 | 0.00191 | 12.47 | 19.67 | 0.63 | EME2 | 197342 | NM_001010865 | Hs.7247 | ENSG00000197774 |
| 0.0001585 | 0.00195 | 35.56 | 53.72 | 0.66 | TMC8 | 147138 | NM_152468 | Hs.592102 | ENSG00000167895 |
| 0.0001648 | 0.00202 | 43.8 | 24.13 | 1.82 | C9orf16 | 79095 | NM_024112 | Hs.522412 | ENSG00000171159 |
| 0.0001667 | 0.00204 | 24.78 | 11.77 | 2.11 | PALM2AKAP2 | 445815 | | | |
| 0.0001726 | 0.0021 | 22.13 | 36.24 | 0.61 | ARHGEF10L | 55160 | NM_001011722 | Hs.443460 | ENSG00000074964 |
| 0.0001746 | 0.00212 | 47.94 | 74.68 | 0.64 | ARID3A | 1820 | NM_005224 | Hs.501296 | ENSG00000116017 |
| 0.000178 | 0.00216 | 10.89 | 6.92 | 1.57 | SWT1 | 54823 | NM_001105518 | Hs.134183 | ENSG00000116668 |
| 0.0001806 | 0.00219 | 8.42 | 5.56 | 1.51 | MPP6 | 51678 | NM_001303037 | Hs.533355 | ENSG00000105926 |
| 0.0001988 | 0.00236 | 51.12 | 79.99 | 0.64 | ADAM19 | 8728 | NM_023038 | Hs.483944 | ENSG00000135074 |
| 0.000202 | 0.00239 | 11.79 | 17.73 | 0.66 | SAMD1 | 90378 | NM_138352 | Hs.140309 | ENSG00000141858 |
| 0.0002085 | 0.00245 | 9.28 | 5.65 | 1.64 | CCDC175 | 729665 | NM_001164399 | Hs.29706 | ENSG00000151838 |
| 0.0002103 | 0.00247 | 15.9 | 26.12 | 0.61 | SCRIB | 23513 | NM_015356 | Hs.436329 | ENSG00000180900 |
| 0.0002252 | 0.00261 | 9.26 | 5.36 | 1.73 | AC024940.2 | | | | |
| 0.00023 | 0.00266 | 11.08 | 6.5 | 1.71 | CD109 | 135228 | NM_001159587 | Hs.399891 | ENSG00000156535 |
| 0.0002308 | 0.00267 | 41.02 | 26.36 | 1.56 | NID1 | 4811 | NM_002508 | Hs.356624 | ENSG00000116962 |
| 0.0002347 | 0.00271 | 53.88 | 35.64 | 1.51 | SNHG6 | 641638 | NR_002599 | Hs.372680 | ENSG00000245910 |
| 0.000237 | 0.00272 | 7.58 | 13.4 | 0.57 | CSGALNACT1 | 55790 | NM_001130518 | Hs.613729 | ENSG00000147408 |
| 0.0002444 | 0.00279 | 9 | 14.32 | 0.63 | TMEM150B | 284417 | NM_001085488 | Hs.382625 | ENSG00000180061 |
| 0.0002452 | 0.0028 | 10.14 | 6.27 | 1.62 | RNVU1-25 | 115409981 | | | |
| 0.0002492 | 0.00283 | 12.2 | 19.13 | 0.64 | TDRD9 | 122402 | NM_153046 | Hs.21454 | ENSG00000156414 |
| 0.0003038 | 0.00333 | 13.1 | 6.54 | 2 | MAGI2-AS3 | 100505881 | NR_038343 | Hs.31474 | ENSG00000234456 |
| 0.0003062 | 0.00335 | 8.96 | 14.3 | 0.63 | PWWP2B | 170394 | NM_001098637 | Hs.527751 | ENSG00000171813 |
| 0.0003404 | 0.00367 | 93.7 | 51.33 | 1.83 | LMNA | 4000 | NM_001257374 | Hs.594444 | ENSG00000160789 |
| 0.0003702 | 0.00393 | 1848.19 | 2846.67 | 0.65 | FOS | 2353 | NM_005252 | Hs.25647 | ENSG00000170345 |
| 0.000398 | 0.00417 | 16.14 | 25.85 | 0.62 | MARVELD1 | 83742 | NM_031484 | Hs.744073 | ENSG00000155254 |
| 0.0004018 | 0.0042 | 9.84 | 6.06 | 1.62 | MTRNR2L12 | 100463498 | | | |
| 0.0004088 | 0.00426 | 22.45 | 34.49 | 0.65 | SLC19A1 | 6573 | NM_001205206 | Hs.84190 | ENSG00000173638 |
| 0.0004213 | 0.00437 | 102.75 | 68.38 | 1.5 | ATP5F1E | 514 | NM_006886 | Hs.177530 | ENSG00000124172 |
| 0.0004263 | 0.0044 | 12.8 | 20.33 | 0.63 | ERLIN1 | 10613 | NM_001100626 | Hs.150087 | ENSG00000107566 |
| 0.0004305 | 0.00443 | 20.41 | 38.35 | 0.53 | GIMAP6 | 474344 | NM_001007224 | Hs.647105 | ENSG00000133561 |
| 0.0004402 | 0.00452 | 42.34 | 69.45 | 0.61 | SLC12A7 | 10723 | NM_006598 | Hs.172613 | ENSG00000113504 |
| 0.0004542 | 0.00463 | 10.52 | 19.73 | 0.53 | KCNE1 | 3753 | NM_000219 | Hs.121495 | ENSG00000180509 |
| 0.0004556 | 0.00465 | 26.83 | 40.55 | 0.66 | BCL2L13 | 23786 | NM_001270726 | Hs.631672 | ENSG00000099968 |
| 0.0004565 | 0.00465 | 15.32 | 9.5 | 1.61 | SH3PXD2A | 9644 | NM_014631 | Hs.678727 | ENSG00000107957 |
| 0.0004593 | 0.00467 | 13.26 | 7.16 | 1.85 | RNY3 | 6085 | NR_004392 | | ENSG00000202354 |
| 0.0004647 | 0.00471 | 32.21 | 48.45 | 0.66 | IL27RA | 9466 | NM_004843 | Hs.132781 | ENSG00000104998 |
| 0.000497 | 0.00498 | 65.61 | 116.26 | 0.56 | SH3TC1 | 54436 | NM_001318480 | Hs.479116 | ENSG00000125089 |
| 0.0005569 | 0.00551 | 10.73 | 16.3 | 0.66 | C5AR2 | 27202 | NM_001271749 | Hs.534412 | ENSG00000134830 |
| 0.0005884 | 0.00577 | 9.53 | 6.36 | 1.5 | AC083862.2 | | | | |
| 0.0005978 | 0.00584 | 56.91 | 94.47 | 0.6 | PADI2 | 11240 | NM_007365 | Hs.33455 | ENSG00000117115 |
| 0.0006075 | 0.00592 | 8.56 | 5.56 | 1.54 | HEXIM2 | 124790 | NM_001303436 | Hs.56382 | ENSG00000168517 |
| 0.0006118 | 0.00595 | 13.59 | 20.95 | 0.65 | RCN3 | 57333 | NM_020650 | Hs.567550 | ENSG00000142552 |
| 0.0006178 | 0.006 | 11.87 | 19.66 | 0.6 | PEAK3 | 374872 | NM_198532 | Hs.511803 | ENSG00000188305 |
| 0.0007045 | 0.00667 | 40.73 | 15.17 | 2.69 | DDX11L10 | 100287029 | NR_045117 | Hs.592089 | ENSG00000233614 |
| 0.0007213 | 0.00679 | 16.16 | 24.92 | 0.65 | ITGB2-AS1 | 100505746 | NR_038311 | Hs.661035 | ENSG00000227039 |
| 0.0007331 | 0.00688 | 12.37 | 19.84 | 0.62 | VNN3 | 55350 | NM_001291702 | Hs.183656 | ENSG00000093134 |
| 0.0007483 | 0.00698 | 21.41 | 14.12 | 1.52 | JKAMP | 51528 | NM_001098625 | Hs.446850 | ENSG00000050130 |
| 0.0007666 | 0.00712 | 9.94 | 6.13 | 1.62 | DHRS3 | 9249 | NM_001319225 | Hs.289347 | ENSG00000162496 |
| 0.0007931 | 0.00733 | 9.6 | 6.1 | 1.57 | SKA3 | 221150 | NM_001166017 | Hs.88523 | ENSG00000165480 |
| 0.0007939 | 0.00733 | 19.73 | 33.62 | 0.59 | MMP25 | 64386 | NM_004142 | Hs.654979 | ENSG00000008516 |
| 0.0008146 | 0.00751 | 46.08 | 8.57 | 5.38 | HBB | 3043 | NM_000518 | Hs.523443 | ENSG00000244734 |
| 0.0009156 | 0.00826 | 21.62 | 34.29 | 0.63 | ERF | 2077 | NM_001301035 | Hs.655969 | ENSG00000105722 |
| 0.0009432 | 0.00848 | 19.13 | 9.47 | 2.02 | TNF | 7124 | NM_000594 | Hs.241570 | ENSG00000232810 |

A heatmap of the data shown in Table 2 is provided in FIG. 26.

TABLE 3
Differentially Expressed Genes (DGE) that are potentially within the IBD GWAS
loci.

| | | Geom | Geom | | | | | | |
|---|---|---|---|---|---|---|---|---|---|
| | | mean | mean of | | | | | | |
| | | of | intensities | | | | | | |
| Parametric | | intensities | PAF- | Fold- | | Entrez | | | |
| p-value | FDR | Cdmono | Cdmono | change | UniqueID | ID | Accession | UGCluster | Ensembl |
| <1e−07 | <1e−07 | 26.78 | 7.02 | 3.81 | TRAPPC3L | 1E+08 | NM_001139444 | Hs.134795 | ENSG00000173626 |
| <1e−07 | <1e−07 | 104.95 | 28.87 | 3.64 | SELP | 6403 | NM_003005 | Hs.73800 | ENSG00000174175 |
| <1e−07 | <1e−07 | 250.44 | 80.62 | 3.11 | CD226 | 10666 | NM_001303618 | Hs.660130 | ENSG00000150637 |
| <1e−07 | <1e−07 | 118.11 | 36.29 | 3.25 | ACRBP | 84519 | NM_032489 | Hs.123239 | ENSG00000111644 |
| <1e−07 | <1e−07 | 43.3 | 13.04 | 3.32 | BANK1 | 55024 | NM_001083907 | Hs.480400 | ENSG00000153064 |
| <1e−07 | <1e−07 | 647.05 | 172 | 3.76 | PF4 | 5196 | NM_002619 | Hs.81564 | ENSG00000163737 |
| <1e−07 | <1e−07 | 119.65 | 55.5 | 2.16 | AGPAT1 | 10554 | NM_006411 | Hs.409230 | ENSG00000204310 |
| <1e−07 | <1e−07 | 1662.12 | 562.73 | 2.95 | PPBP | 5473 | NM_002704 | Hs.2164 | ENSG00000163736 |
| <1e−07 | <1e−07 | 85.07 | 37.59 | 2.26 | NDUFAF3 | 25915 | NM_199069 | Hs.31387 | ENSG00000178057 |
| <1e−07 | <1e−07 | 63.27 | 15.81 | 4 | CXCL5 | 6374 | NM_002994 | Hs.89714 | ENSG00000163735 |
| <1e−07 | <1e−07 | 67.33 | 30.84 | 2.18 | SMIM3 | 85027 | NM_032947 | Hs.29444 | ENSG00000256235 |
| <1e−07 | <1e−07 | 10.89 | 5.33 | 2.04 | PKIG | 11142 | NM_001281444 | Hs.472831 | ENSG00000168734 |
| <1e−07 | <1e−07 | 597.44 | 342.76 | 1.74 | CALM3 | 808 | NM_001329921 | Hs.515487 | ENSG00000160014 |
| <1e−07 | <1e−07 | 19.66 | 7.24 | 2.72 | CTSW | 1521 | NM_001335 | Hs.416848 | ENSG00000172543 |
| <1e−07 | <1e−07 | 35.83 | 21.13 | 1.7 | RBX1 | 9978 | NM_014248 | Hs.474949 | ENSG00000100387 |
| <1e−07 | <1e−07 | 121.99 | 73.88 | 1.65 | DAP | 1611 | NM_001291963 | Hs.75189 | ENSG00000112977 |
| <1e−07 | <1e−07 | 138.81 | 89.39 | 1.55 | TMBIM1 | 64114 | NM_001321427 | Hs.591605 | ENSG00000135926 |
| <1e−07 | <1e−07 | 131.37 | 84.69 | 1.55 | SLAIN2 | 57606 | NM_020846 | Hs.479677 | ENSG00000109171 |
| <1e−07 | <1e−07 | 128.76 | 86.8 | 1.48 | CDC42SE2 | 56990 | NM_001038702 | Hs.508829 | ENSG00000158985 |
| <1e−07 | <1e−07 | 66.37 | 108.37 | 0.61 | NACC2 | 138151 | NM_144653 | Hs.112895 | ENSG00000148411 |
| <1e−07 | <1e−07 | 21.7 | 11.95 | 1.82 | CAMTA1 | 23261 | NM_001195563 | Hs.397705 | ENSG00000171735 |
| <1e−07 | <1e−07 | 9.44 | 5.29 | 1.78 | PRKCQ | 5588 | NM_001242413 | Hs.498570 | ENSG00000065675 |
| <1e−07 | <1e−07 | 48.79 | 75.92 | 0.64 | NCF4 | 4689 | NM_000631 | Hs.474781 | ENSG00000100365 |
| <1e−07 | <1e−07 | 128.73 | 53.12 | 2.42 | NEXN | 91624 | NM_001172309 | Hs.612385 | ENSG00000162614 |
| 1.00E−07 | 2.21E−06 | 295.82 | 469.81 | 0.63 | APOBR | 55911 | NM_018690 | Hs.200333 | ENSG00000184730 |
| 1.00E−07 | 2.21E−06 | 23.99 | 12.3 | 1.95 | ORMDL3 | 94103 | NM_001320801 | Hs.514151 | ENSG00000172057 |
| 2.00E−07 | 4.26E−06 | 137.93 | 211.32 | 0.65 | ADCY7 | 113 | NM_001114 | Hs.513578 | ENSG00000121281 |
| 3.00E−07 | 6.16E−06 | 9.67 | 5.6 | 1.73 | SLC22A23 | 63027 | NM_001286455 | Hs.713588 | ENSG00000137266 |
| 5.00E−07 | 9.91E−06 | 67.81 | 96.03 | 0.71 | ZSWIM8 | 23053 | NM_001242487 | Hs.65135 | ENSG00000214655 |
| 6.00E−07 | 1.15E−05 | 603.95 | 376.5 | 1.6 | GPX1 | 2876 | NM_000581 | Hs.76686 | ENSG00000233276 |
| 8.00E−07 | 1.48E−05 | 63.16 | 78.47 | 0.8 | ATF6B | 1388 | NM_001136153 | Hs.42853 | ENSG00000213676 |
| 3.10E−06 | 5.57E−05 | 164.34 | 206.41 | 0.8 | SEC16A | 9919 | NM_001276418 | Hs.522500 | ENSG00000148396 |
| 3.60E−06 | 6.27E−05 | 61.2 | 92.59 | 0.66 | SLC12A9 | 56996 | NM_001267812 | Hs.521087 | ENSG00000146828 |
| 3.80E−06 | 6.43E−05 | 75.93 | 109.61 | 0.69 | LTBR | 4055 | NM_001270987 | Hs.1116 | ENSG00000111321 |
| 5.60E−06 | 9.20E−05 | 364.49 | 528.87 | 0.69 | LSP1 | 4046 | NM_001013253 | Hs.56729 | ENSG00000130592 |
| 6.10E−06 | 9.74E−05 | 467.51 | 648.62 | 0.72 | NOTCH2 | 4853 | NM_001200001 | Hs.487360 | ENSG00000134250 |
| 7.50E−06 | 0.000117 | 16.15 | 23.83 | 0.68 | MUS81 | 80198 | NM_001350283 | Hs.288798 | ENSG00000172732 |
| 8.10E−06 | 0.000123 | 118.96 | 158.63 | 0.75 | TSPAN14 | 81619 | NM_001128309 | Hs.718943 | ENSG00000108219 |
| 9.30E−06 | 0.000137 | 183.71 | 137.96 | 1.33 | TAB2 | 23118 | NM_001292034 | Hs.269775 | ENSG00000055208 |
| 1.38E−05 | 0.000198 | 191.66 | 247.95 | 0.77 | CCDC69 | 26112 | NM_015621 | Hs.655336 | ENSG00000198624 |
| 1.60E−05 | 0.00022 | 19.9 | 28.74 | 0.69 | PFKFB4 | 5210 | NM_001317134 | Hs.476217 | ENSG00000114268 |
| 1.61E−05 | 0.00022 | 203.1 | 290.68 | 0.7 | PTK2B | 2185 | NM_004103 | Hs.491322 | ENSG00000120899 |
| 2.00E−05 | 0.000267 | 27.43 | 42.8 | 0.64 | CARD9 | 64170 | NM_052813 | Hs.694071 | ENSG00000187796 |
| 2.20E−05 | 0.000288 | 231.93 | 289.85 | 0.8 | PRRC2A | 7916 | NM_004638 | Hs.123239 | ENSG00000204469 |
| 2.39E−05 | 0.000305 | 55.32 | 40.65 | 1.36 | PARK7 | 11315 | NM_001123377 | Hs.419640 | ENSG00000116288 |
| 2.74E−05 | 0.000342 | 126.44 | 208.39 | 0.61 | CCDC88B | 283234 | NM_032251 | Hs.98564 | ENSG00000168071 |
| 3.44E−05 | 0.000417 | 23.05 | 12.9 | 1.79 | PTGIR | 5739 | NM_000960 | Hs.458324 | ENSG00000160013 |
| 3.48E−05 | 0.000417 | 132.1 | 192.23 | 0.69 | TYK2 | 7297 | NM_003331 | Hs.75516 | ENSG00000105397 |
| 4.66E−05 | 0.000547 | 105.6 | 162.89 | 0.65 | SBNO2 | 22904 | NM_001100122 | Hs.408708 | ENSG00000064932 |
| 5.87E−05 | 0.000675 | 27.97 | 38.38 | 0.73 | APEH | 327 | NM_001640 | Hs.517969 | ENSG00000164062 |
| 6.31E−05 | 0.000711 | 11.75 | 18.2 | 0.65 | SNAPC4 | 6621 | NM_003086 | Hs.113265 | ENSG00000165684 |
| 6.52E−05 | 0.000721 | 59.27 | 82.17 | 0.72 | NOD2 | 64127 | NM_001293557 | Hs.592072 | ENSG00000167207 |
| 8.09E−05 | 0.000878 | 24.08 | 38.64 | 0.62 | USP19 | 10869 | NM_001199160 | Hs.255596 | ENSG00000172046 |
| 8.86E−05 | 0.000943 | 49.64 | 66.82 | 0.74 | UQCRC1 | 7384 | NM_003365 | Hs.119251 | ENSG00000010256 |
| 9.59E−05 | 0.001 | 13.23 | 9.65 | 1.37 | TCTA | 6988 | NM_022171 | Hs.517962 | ENSG00000145022 |
| 0.0001002 | 0.00102 | 108.05 | 150.36 | 0.72 | NOTCH1 | 4851 | NM_017617 | Hs.495473 | ENSG00000148400 |
| 0.0001031 | 0.00102 | 17.17 | 12.59 | 1.36 | SLC50A1 | 55974 | NM_001122837 | Hs.292154 | ENSG00000169241 |
| 0.0001031 | 0.00102 | 142.22 | 110.78 | 1.28 | LPP | 4026 | NM_001167671 | Hs.720220 | ENSG00000145012 |
| 0.0001234 | 0.0012 | 53.81 | 64.67 | 0.83 | CRTC3 | 64784 | NM_001042574 | Hs.567572 | ENSG00000140577 |
| 0.0001271 | 0.00122 | 252.91 | 170.31 | 1.48 | CCND3 | 896 | NM_001136017 | Hs.534307 | ENSG00000112576 |
| 0.0001309 | 0.00123 | 207.32 | 152.55 | 1.36 | SKAP2 | 8935 | NM_001303468 | Hs.200770 | ENSG00000005020 |
| 0.0001341 | 0.00124 | 33.78 | 43.55 | 0.78 | SNX17 | 9784 | NM_001267059 | Hs.278569 | ENSG00000115234 |
| 0.0001434 | 0.00131 | 116.22 | 152.23 | 0.76 | ICAM3 | 3385 | NM_001320605 | Hs.654563 | ENSG00000076662 |
| 0.0001484 | 0.00133 | 68.17 | 49.89 | 1.37 | UBE2L3 | 7332 | NM_001256355 | Hs.108104 | ENSG00000185651 |
| 0.0002057 | 0.00182 | 45.09 | 58.66 | 0.77 | EVI5 | 7813 | NM_001308248 | Hs.594434 | ENSG00000067208 |
| 0.0002208 | 0.00192 | 9.83 | 13.41 | 0.73 | CLN3 | 1201 | NM_000086 | Hs.534667 | ENSG00000188603 |
| 0.0002384 | 0.00202 | 47.28 | 59.51 | 0.79 | ACO2 | 50 | NM_001098 | Hs.643610 | ENSG00000100412 |
| 0.0002394 | 0.00202 | 9.66 | 14.15 | 0.68 | SLC26A6 | 65010 | NM_001040454 | Hs.631925 | ENSG00000225697 |
| 0.0002651 | 0.00221 | 194.54 | 228.84 | 0.85 | ATXN2L | 11273 | NM_001308230 | Hs.460499 | ENSG00000168488 |
| 0.0003022 | 0.00248 | 56.51 | 75.49 | 0.75 | KSR1 | 8844 | NM_014238 | Hs.133534 | ENSG00000141068 |
| 0.0003559 | 0.00288 | 29.72 | 40.11 | 0.74 | SKIV2L | 6499 | NM_006929 | Hs.89864 | ENSG00000204351 |
| 0.0003702 | 0.00294 | 1848.19 | 2846.67 | 0.65 | FOS | 2353 | NM_005252 | Hs.25647 | ENSG00000170345 |
| 0.0003734 | 0.00294 | 6.81 | 5 | 1.36 | DOK6 | 220164 | NM_152721 | Hs.278285 | ENSG00000206052 |
| 0.0004118 | 0.00319 | 7.2 | 5.14 | 1.4 | C1orf112 | 55732 | NM_001320047 | Hs.443551 | ENSG00000000460 |
| 0.0004163 | 0.00319 | 41.06 | 51.4 | 0.8 | ZC3H7B | 23264 | NM_017590 | Hs.592188 | ENSG00000100403 |
| 0.000422 | 0.00319 | 20.62 | 30.42 | 0.68 | PLCB3 | 5331 | NM_000932 | Hs.523761 | ENSG00000149782 |
| 0.0004852 | 0.00362 | 19.49 | 27.4 | 0.71 | NELFE | 7936 | NM_002904 | Hs.423935 | ENSG00000204356 |
| 0.000513 | 0.00378 | 21.59 | 29.06 | 0.74 | TCOF1 | 6949 | NM_000356 | Hs.519672 | ENSG00000070814 |
| 0.0005387 | 0.00392 | 123 | 156.18 | 0.79 | MTMR3 | 8897 | NM_001013676 | Hs.474536 | ENSG00000100330 |
| 0.0005521 | 0.00397 | 19.2 | 13.33 | 1.44 | NCKIPSD | 51517 | NM_016453 | Hs.655006 | ENSG00000213672 |
| 0.0005629 | 0.004 | 162.99 | 132.01 | 1.23 | SERINC3 | 10955 | NM_006811 | Hs.272168 | ENSG00000132824 |
| 0.0007465 | 0.00523 | 6.79 | 5.21 | 1.3 | DNAJB4 | 11080 | NM_001317099 | Hs.13852 | ENSG00000162616 |
| 0.0008189 | 0.00567 | 118.05 | 145.12 | 0.81 | BAG6 | 7917 | NM_001098534 | Hs.440900 | ENSG00000204463 |
| 0.000943 | 0.00638 | 6.92 | 5.17 | 1.34 | LY6G5C | 80741 | NM_001002848 | Hs.25738 | ENSG00000204428 |
| 0.0009432 | 0.00638 | 19.13 | 9.47 | 2.02 | TNF | 7124 | NM_000594 | Hs.241570 | ENSG00000232810 |

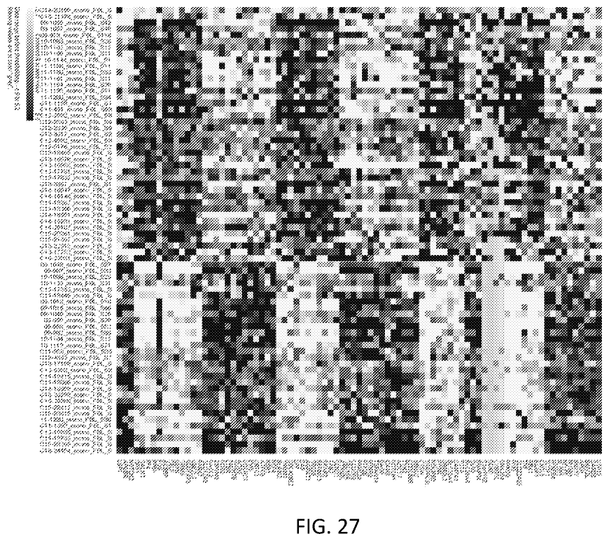

A heat map of the data shown in Table 3 is provide in FIG. 27.

Tables 4, 5, and 6. Gene Ontology analysis of various biological processes.

TABLE 4

| | Observed in | | |
|---|---|---|---|
| | selected | Expected in selected | |
| GO Term | subset | subset | Observed/Expected |
| sperm part | 5 | 1.66 | 3.01 |

TABLE 5

| | Observed in | Expected in | |
|---|---|---|---|
| | selected | selected | |
| GO Term | subset | subset | Observed/Expected |
| transcription factor | 5 | 1.19 | 4.21 |
| activity, RNA | | | |
| polymerase II | | | |
| transcription factor | | | |
| binding | | | |
| chemokine receptor | 8 | 2.37 | 3.37 |
| binding | | | |
| cytokine activity | 5 | 1.48 | 3.37 |
| carboxylic acid | 6 | 2.08 | 2.89 |
| transmembrane | | | |
| transporter activity | | | |
| organic acid | 6 | 2.08 | 2.89 |
| transmembrane | | | |
| transporter activity | | | |
| receptor ligand | 6 | 2.23 | 2.7 |
| activity | | | |
| receptor regulator | 6 | 2.23 | 2.7 |
| activity | | | |
| lyase activity | 6 | 2.23 | 2.7 |
| chloride | 5 | 1.93 | 2.59 |
| transmembrane | | | |
| transporter activity | | | |
| enzyme activator | 9 | 3.71 | 2.43 |
| activity | | | |
| G-protein coupled | 9 | 4.15 | 2.17 |
| receptor binding | | | |
| cell adhesion | 8 | 3.86 | 2.07 |
| molecule binding | | | |
| protein tyrosine | 7 | 3.41 | 2.05 |
| kinase activity | | | |

TABLE 6

| | Observed | Expected in | |
|---|---|---|---|
| | in selected | selected | |
| GO Term | subset | subset | Observed/Expected |
| positive regulation of | 9 | 1.8 | 5.01 |
| oxidoreductase activity | | | |
| positive regulation of | 5 | 1 | 5.01 |
| lipid metabolic process | | | |
| protein deglycation | 5 | 1 | 5.01 |
| protein repair | 5 | 1 | 5.01 |
| intrinsic apoptotic | 5 | 1 | 5.01 |
| signaling pathway | | | |
| in response to | | | |
| oxidative stress | | | |
| nucleotide-excision | 9 | 2 | 4.51 |
| repair | | | |
| regulation of release | 5 | 1.2 | 4.17 |
| of sequestered calcium | | | |
| ion into cytosol | | | |
| cellular aldehyde | 5 | 1.2 | 4.17 |
| metabolic process | | | |
| negative regulation of | 6 | 1.6 | 3.76 |
| oxidative stress-induced | | | |
| cell death | | | |
| negative regulation of | 6 | 1.6 | 3.76 |
| response to oxidative | | | |
| stress | | | |
| regulation of response to | 9 | 2.4 | 3.76 |
| oxidative stress | | | |
| negative regulation of | 6 | 1.6 | 3.76 |
| cellular response to | | | |
| oxidative stress | | | |
| regulation of cellular | 9 | 2.4 | 3.76 |
| response to oxidative | | | |
| stress | | | |
| endothelial cell | 6 | 1.6 | 3.76 |
| differentiation | | | |
| aerobic respiration | 6 | 1.6 | 3.76 |
| cell death in response | 8 | 2.2 | 3.64 |
| to oxidative stress | | | |
| response to muramyl | 8 | 2.2 | 3.64 |
| dipeptide | | | |
| regulation of response | 5 | 1.4 | 3.58 |
| to reactive oxygen | | | |
| species | | | |
| positive regulation of | 5 | 1.4 | 3.58 |
| nucleotide metabolic | | | |
| process | | | |
| regulation of calcium | 5 | 1.4 | 3.58 |
| ion transport into | | | |
| cytosol | | | |
| extrinsic apoptotic | 5 | 1.4 | 3.58 |
| signaling pathway | | | |
| via death domain | | | |
| receptors | | | |
| regulation of oxidative | 7 | 2 | 3.51 |
| stress-induced | | | |
| cell death | | | |
| regulation of | 9 | 2.6 | 3.47 |
| oxidoreductase activity | | | |
| tricarboxylic acid | 6 | 1.8 | 3.34 |
| metabolic process | | | |
| detoxification | 7 | 2.2 | 3.19 |
| lytic vacuole | 5 | 1.6 | 3.13 |
| organization | | | |
| lysosome organization | 5 | 1.6 | 3.13 |
| citrate metabolic | 5 | 1.6 | 3.13 |
| process | | | |
| heart valve | 5 | 1.6 | 3.13 |
| morphogenesis | | | |
| heart valve development | 5 | 1.6 | 3.13 |
| positive regulation | 8 | 2.6 | 3.08 |
| of small molecule | | | |
| metabolic process | | | |
| ceramide metabolic | 8 | 2.6 | 3.08 |
| process | | | |
| regulation of neuron | 7 | 2.4 | 2.92 |
| apoptotic process | | | |
| chloride transport | 7 | 2.4 | 2.92 |
| positive regulation | 7 | 2.4 | 2.92 |
| of leukocyte mediated | | | |
| immunity | | | |
| positive regulation of | 8 | 2.8 | 2.86 |
| stress-activated | | | |
| protein kinase | | | |
| signaling cascade | | | |
| positive regulation | 8 | 2.8 | 2.86 |
| of stress-activated | | | |
| MAPK cascade | | | |
| response to metal ion | 8 | 2.8 | 2.86 |
| JNK cascade | 9 | 3.2 | 2.82 |
| cellular detoxification | 5 | 1.8 | 2.78 |
| coronary vasculature | 5 | 1.8 | 2.78 |
| development | | | |
| negative regulation | 5 | 1.8 | 2.78 |
| of sequestering | | | |
| of calcium ion | | | |
| regulation of | 5 | 1.8 | 2.78 |
| sequestering of | | | |
| calcium ion | | | |
| release of sequestered | 5 | 1.8 | 2.78 |
| calcium ion into | | | |
| cytosol | | | |
| sequestering of | 5 | 1.8 | 2.78 |
| calcium ion | | | |
| negative regulation | 5 | 1.8 | 2.78 |
| of neuron | | | |
| apoptotic process | | | |
| cardiac muscle | 6 | 2.2 | 2.73 |
| tissue development | | | |
| positive regulation | 6 | 2.2 | 2.73 |
| of JNK cascade | | | |
| endothelium | 6 | 2.2 | 2.73 |
| development | | | |
| positive regulation | 6 | 2.2 | 2.73 |
| of lymphocyte | | | |
| mediated immunity | | | |
| negative regulation | 7 | 2.6 | 2.7 |
| of extrinsic | | | |
| apoptotic signaling | | | |
| pathway | | | |
| neuron apoptotic | 7 | 2.6 | 2.7 |
| process | | | |
| urogenital system | 7 | 2.6 | 2.7 |
| development | | | |
| osteoclast differentiation | 8 | 3 | 2.67 |
| cellular response to | 9 | 3.4 | 2.65 |
| reactive oxygen | | | |
| species | | | |
| negative regulation | 5 | 2 | 2.5 |
| of intrinsic | | | |
| apoptotic signaling | | | |
| pathway | | | |
| regulation of | 8 | 3.2 | 2.5 |
| extrinsic apoptotic | | | |
| signaling pathway | | | |
| inorganic anion | 6 | 2.4 | 2.5 |
| transmembrane | | | |
| transport | | | |
| calcium ion | 5 | 2 | 2.5 |
| transmembrane | | | |
| import into | | | |
| cytosol | | | |
| calcium ion transport | 5 | 2 | 2.5 |
| into cytosol | | | |
| cytosolic calcium | 5 | 2 | 2.5 |
| ion transport | | | |
| cyclic-nucleotide- | 7 | 2.8 | 2.5 |
| mediated signaling | | | |
| cyclic nucleotide | 5 | 2 | 2.5 |
| biosynthetic | | | |
| process | | | |
| regulation of | 6 | 2.4 | 2.5 |
| nucleotide metabolic | | | |
| process | | | |
| cardiac ventricle | 5 | 2 | 2.5 |
| development | | | |
| organic hydroxy | 9 | 3.79 | 2.37 |
| compound | | | |
| biosynthetic process | | | |
| cellular respiration | 9 | 3.79 | 2.37 |
| energy derivation | 9 | 3.79 | 2.37 |
| by oxidation of | | | |
| organic compounds | | | |
| positive regulation | 7 | 3 | 2.34 |
| of immune | | | |
| effector process | | | |
| regulation of JNK | 6 | 2.6 | 2.31 |
| cascade | | | |
| cAMP-mediated | 6 | 2.6 | 2.31 |
| signaling | | | |
| positive regulation | 5 | 2.2 | 2.28 |
| of reactive | | | |
| oxygen | | | |
| species metabolic | | | |
| process | | | |
| regulation of | 5 | 2.2 | 2.28 |
| calcium ion | | | |
| transmembrane | | | |
| transport | | | |
| regulation of | 5 | 2.2 | 2.28 |
| purine nucleotide | | | |
| metabolic process | | | |
| cellular response | 5 | 2.2 | 2.28 |
| to hydrogen | | | |
| peroxide | | | |
| heart contraction | 5 | 2.2 | 2.28 |
| detection of stimulus | 5 | 2.2 | 2.28 |
| negative regulation | 5 | 2.2 | 2.28 |
| of neurogenesis | | | |
| regulation of | 5 | 2.2 | 2.28 |
| dephosphorylation | | | |
| negative regulation | 5 | 2.2 | 2.28 |
| of cell development | | | |
| carbohydrate transport | 5 | 2.2 | 2.28 |
| heart process | 5 | 2.2 | 2.28 |
| mesenchyme | 9 | 3.99 | 2.25 |
| development | | | |
| regulation of | 8 | 3.59 | 2.23 |
| stress-activated | | | |
| protein kinase | | | |
| signaling cascade | | | |
| regulation of | 8 | 3.59 | 2.23 |
| stress-activated | | | |
| MAPK cascade | | | |
| negative regulation | 7 | 3.2 | 2.19 |
| of neuron death | | | |
| regulation of | 7 | 3.2 | 2.19 |
| intracellular pH | | | |
| mesenchymal | 7 | 3.2 | 2.19 |
| cell differentiation | | | |
| response to | 7 | 3.2 | 2.19 |
| hydrogen peroxide | | | |
| regulation of | 7 | 3.2 | 2.19 |
| cellular pH | | | |
| antibiotic | 7 | 3.2 | 2.19 |
| metabolic process | | | |
| cyclic nucleotide | 7 | 3.2 | 2.19 |
| metabolic process | | | |
| regulation of pH | 7 | 3.2 | 2.19 |
| positive regulation | 7 | 3.2 | 2.19 |
| of leukocyte | | | |
| chemotaxis | | | |
| negative regulation | 5 | 2.4 | 2.09 |
| of nervous | | | |
| system | | | |
| development | | | |
| regulation of | 5 | 2.4 | 2.09 |
| lipid biosynthetic | | | |
| process | | | |
| cytokine production | 5 | 2.4 | 2.09 |
| involved in | | | |
| immune response | | | |
| circulatory system | 54 | 26.36 | 2.05 |
| development | | | |
| negative regulation | 9 | 4.39 | 2.05 |
| of proteolysis | | | |
| ERAD pathway | 9 | 4.39 | 2.05 |
| regulation of | 8 | 3.99 | 2 |
| intrinsic apoptotic | | | |
| signaling pathway | | | |
| multicellular | 6 | 3 | 2 |
| organismal | | | |
| homeostasis | | | |
| positive regulation | 8 | 3.99 | 2 |
| of MAP kinase | | | |
| activity | | | |
| defense response | 6 | 3 | 2 |
| to bacterium | | | |
| regulation of | 8 | 3.99 | 2 |
| leukocyte | | | |
| chemotaxis | | | |

In embodiments where more than one biomarker is detected, the differences in expression between a patient having a PAF-CD-mono signature or a CD-mono signature and a reference subject (e.g., non-IBD subject) may be different for each marker, e.g., each of the biomarkers detected is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference sample. In some cases, at least about 80%, 85%, 90%, 95%, 96%, 97%, 98%, or 99% of the biomarkers detected in a transcriptomic signature is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

### Expression and RNA Sequencing Methods

In some embodiments, the gene expression levels in the monocytes (e.g., PAF-CD-mono or CD-mono) of the biological samples may be measured by an array. In some cases, the array comprises a microarray, sequencing, and qPCR. In some instances, the array comprises RNA sequencing (RNA-Seq). In some examples, RNA-Seq data may be analyzed using the BRB array tools. BRB array tools may comprise tools for visualization and statistical analysis of microarray gene expression, copy number, methylation and/or RNA-Seq data. In some examples, the analysis tools may be run using an R-program. In some embodiments, the analysis tools described herein may be used for analyzing genes through differential gene expressions, hierarchical clustering and/or principal component analysis (PCA) plots.

Isolated RNA can be used in hybridization or amplification assays that include, but are not limited to, Southern or Northern analyses, polymerase chain reaction (PCR) analyses and probe arrays. An exemplary method for the determination of RNA levels involves contacting RNA with a nucleic acid molecule (e.g., probe) that can hybridize to the biomarker mRNA. The nucleic acid molecule can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the biomarker genomic DNA. In some embodiments, the RNA is immobilized on a solid surface and contacted with a probe, for example by running the isolated RNA on an agarose gel and transferring the RNA from the gel to a membrane, such as nitrocellulose. In some embodiments, the probe(s) are immobilized on a solid surface, for example, in an Affymetrix gene chip array, and the probe(s) are contacted with RNA.

The level of expression of the biomarker in a sample can also be determined using methods that involve the use of nucleic acid amplification and/or reverse transcriptase, e.g., by RT-PCR, ligase chain reaction, self-sustained sequence replication, transcriptional amplification system, Q-Beta Replicase, rolling circle replication or any other nucleic acid amplification method, followed by the detection of the amplified molecules. These approaches may be useful for the detection of nucleic acid molecules if such molecules are present in very low numbers. In some embodiments, the level of expression of the biomarker is determined by quantitative fluorogenic RT-PCR (e.g., the TaqMan™ System). Such methods may utilize pairs of oligonucleotide primers that are specific for the biomarker.

In some embodiments, biomarker expression is determined by sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, SOLiD sequencing, PacBio sequencing (e.g., SMRT), oxford nanopore technologies sequencing, and sequencing by transient binding (Life Tech). In some cases, next-generation sequencing involves high-throughput sequencing methods. Additional sequencing methods available to one of skill in the art may also be employed.

The expression levels of biomarker RNA can be monitored using a membrane blot (such as used in hybridization analysis such as Northern, Southern, dot, and the like), microwells, sample tubes, gels, beads, fibers, or any solid support comprising bound nucleic acids. The determination of biomarker expression level may also comprise using nucleic acid probes in solution.

In some embodiments, microarrays are used to detect the level of expression of a biomarker. DNA microarrays provide one method for the simultaneous measurement of the expression levels of large numbers of genes. Each array consists of a reproducible pattern of capture probes attached to a solid support. Labeled nucleic acid is hybridized to complementary probes on the array and then detected, e.g., by laser scanning. Hybridization intensities for each probe on the array are determined and converted to a quantitative value representing relative gene expression levels. High-density oligonucleotide arrays may be useful for determining the gene expression profile for a large number of RNAs in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

Nucleic acid polymers include primers useful for amplifying a nucleic acid of biomarker provided herein, e.g., in Tables 1, 2, or 3. For example, for use in an amplification assay such as qPCR. Nucleic acid polymers also include probes comprising a detectable label for detecting and/or quantifying a biomarker of provided herein, e.g., in Tables 1, 2, or 3. In some cases, the probes are reporters that comprise a dye label on one end and a quencher on the other end. When the probes are hybridized to a biomarker nucleic acid, an added DNA polymerase may cleave those hybridized probes, separating the reporter dye from the quencher, and thus increasing fluorescence by the reporter. In some cases, provided is a probe comprising a nucleic acid polymer described herein.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for hybridization to a biomarker of Tables 1, 2, or 3 are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein for determining an expression profile in a subject comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a biomarker nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a biomarker nucleic acid.

### Profile Analysis

The expression profile of a patient sample (test sample) may be compared to a reference sample, e.g., a sample from a subject who does not have IBD such as CD (normal sample). In some cases, a normal sample is that which is or is expected to be free of IBD disease or condition, or a sample that would test negative for any IBD disease or condition. The reference sample may be assayed at the same time, or at a different time from the test sample. In some cases, the expression profile of a reference sample is obtained and stored in a database for comparison to the test sample.

The results of an assay on the test sample may be compared to the results of the same assay on a reference sample. In some cases, the results of the assay on the normal sample are from a database. In some cases, the results of the assay on the normal sample are a known or generally accepted value by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, coy number variations, indications of the presence or absence of one or more DNA markers or regions, and/or nucleic acid sequences.

In some embodiments, the gene expression profile of a test sample is evaluated using methods for correlating gene product expression levels with a specific phenotype of CD, such as the subtype described herein. In some cases, a specified statistical confidence level may be determined in order to provide a diagnostic confidence level. For example, it may be determined that a confidence level of greater than 90% may be a useful predictor of a PAF-CD-mono signature or a CD-mono signature. In other embodiments, more or less stringent confidence levels may be chosen. For example, a confidence level of approximately 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and the number of gene expression products analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operator Curve analysis (ROC), binormal ROC, principal component analysis, partial least squares analysis, singular value decomposition, least absolute shrinkage and selection operator analysis, least angle regression, and the threshold gradient directed regularization method.

Raw gene expression level data may in some cases be improved through the application of algorithms designed to normalize and or improve the reliability of the data. In some embodiments of the present invention the data analysis requires a computer or other device, machine or apparatus for application of the various algorithms described herein due to the large number of individual data points that are processed. A “machine learning algorithm” refers to a computational-based prediction methodology, also known as a “classifier”, employed for characterizing a gene expression profile. The signals corresponding to certain expression levels, which are obtained by, e.g., microarray-based hybridization assays or sequencing, are typically subjected to the algorithm in order to classify the expression profile. Supervised learning generally involves “training” a classifier to recognize the distinctions among classes and then “testing” the accuracy of the classifier on an independent test set. For test samples the classifier can be used to predict the class in which the samples belong.

In some cases, the robust multi-array Average (RMA) method may be used to normalize the raw data. The RMA method begins by computing background-corrected intensities for each matched cell on a number of microarrays. The background corrected values are restricted to positive values as described by Irizarry et al. Biostatistics 2003 Apr. 4 (2): 249-64. The background corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Data may further be filtered to remove data that may be considered suspect. In some embodiments, data deriving from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some cases, unreliable probe sets may be selected for exclusion from data analysis by ranking probe-set reliability against a series of reference datasets. For example, RefSeq or Ensembl (EMBL) are considered very high-quality reference datasets. Data from probe sets matching RefSeq or Ensembl sequences may in some cases be specifically included in microarray analysis experiments due to their expected high reliability. Similarly, data from probe-sets matching less reliable reference datasets may be excluded from further analysis, or considered on a case-by-case basis for inclusion. In some cases, the Ensembl high throughput cDNA (HTC) and/or mRNA reference datasets may be used to determine the probe-set reliability separately or together. In other cases, probe-set reliability may be ranked. For example, probes and/or probe-sets that match perfectly to all reference datasets such as for example RefSeq, HTC, and mRNA, may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there are many methods understood in the art for assessing the reliability of a given probe and/or probe-set for molecular profiling and the methods of the present invention encompass any of these methods and combinations thereof.

The results of the expression profile may be analyzed to classify a subject as having or lacking an IBD disease or condition. In some cases, a diagnostic result may indicate a certain molecular pathway involved in the IBD disease or condition, or a certain grade or stage of a particular IBD disease or condition. In some cases, a diagnostic result may inform an appropriate therapeutic intervention, such as a specific drug regimen like a molecule that targets a biomolecule in a pathway of any biomarker provided herein, e.g., in Tables 1, 2, or 3, or a surgical intervention. In some cases, a diagnostic result indicates suitability or non-suitability of a patient for treatment with anti-TNFα.

In some embodiments, results are classified using a trained algorithm. Trained algorithms include algorithms that have been developed using a reference set of samples with a known IBD phenotype, such as PAF-CD-mono, or with an inflammatory monocyte signature or a CD signature. Algorithms suitable for categorization of samples include but are not limited to k-nearest neighbor algorithms, concept vector algorithms, naive Bayesian algorithms, neural network algorithms, hidden Markov model algorithms, genetic algorithms, and mutual information feature selection algorithms or any combination thereof. In some cases, trained algorithms may incorporate data other than gene expression such as DNA polymorphism data, sequencing data, scoring or diagnosis by cytologists or pathologists, information provided by the pre-classifier algorithm, or information about the medical history of the subject.

In some embodiments, described herein are methods for diagnosing a disease or a condition disclosed herein. In some embodiments, methods comprise: (a) detecting an increase or a decrease in expression of one or more genes provided in Tables 1, 2, or 3; and (b) diagnosing the subject with the disease or the condition based on the expression of the one or more genes that is detected. In some embodiments, methods comprise detecting a transcriptomic signature of the subject, wherein the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Tables 1, 2, and 3. In some embodiments, the transcriptomic signature comprises an increase or a decrease in any combination of 1, 2, 3, 4, 5, 6, 7, 8 genes selected from CARD9, NOD2, TNFRSF1B, CD93, SELL, ITGAL, ITGAM, and IGTB2. In some embodiments, the transcriptomic signature comprises an increase or a decrease in any combination of 1, 2, 3, 4, 5, 6, 7, 8 genes selected from TLR1, TLR5, TRLR6, TRAF3, TLR8, TLR4, MYD88, and IRAK3. In some embodiments, the transcriptomic signature comprises the transcriptomic signature comprises an increase or a decrease in VWF. In embodiments, the transcriptomic signature comprises an increase or a decrease in any combination of 1, 2, 3, 4, 5, 6, 7, or 8 genes selected from CLU, ITGA2B, MGLL, PROS1, GP1BA, GP9, SPARC, and YES1. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

In certain embodiments, described herein are methods for evaluating an effect of a treatment described herein. In some instances, the treatment comprises administration with a therapeutic agent provided herein, and optionally one or more additional therapeutic agents. In some instances, the treatment is monitored by evaluating the gene expression profile of a subject for expression of one or more genes disclosed herein, e.g., in Tables 1, 2, and 3. The gene expression profile may be determined prior to and/or after administration of a therapeutic agent. Gene expression profiling may also be used to ascertain the potential efficacy of a specific therapeutic intervention prior to administering to a subject. In some embodiments, methods comprise detecting a transcriptomic signature of the subject, wherein the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Tables 1, 2, and 3. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein.

In some embodiments, methods disclosed herein comprise treating a disease or a condition in a subject by administering a therapeutic agent to the subject, provided that a transcriptomic signature disclosed herein is detected in a sample obtained from the subject. In some embodiments, the transcriptomic signature comprises an increase or a decrease in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Tables 1, 2, and 3. In some embodiments, the transcriptomic signature comprises an increase or a decrease in any combination of 1, 2, 3, 4, 5, 6, 7, 8 genes selected from CARD9, NOD2, TNFRSF1B, CD93, SELL, ITGAL, ITGAM, and IGTB2. In some embodiments, the transcriptomic signature comprises an increase or a decrease in any combination of 1, 2, 3, 4, 5, 6, 7, 8 genes selected from TLR1, TLR5, TRLR6, TRAF3, TLR8, TLR4, MYD88, and IRAK3. In some embodiments, the transcriptomic signature comprises the transcriptomic signature comprises an increase or a decrease in VWF. In embodiments, the transcriptomic signature comprises an increase or a decrease in any combination of 1, 2, 3, 4, 5, 6, 7, or 8 genes selected from CLU, ITGA2B, MGLL, PROS1, GP1BA, GP9, SPARC, and YES1. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold as compared to a reference sample. In some embodiments, the increase or the decrease in the expression of the one or more genes disclosed herein is an increase or a decrease of at least about 80%, 85%, 90%, 95%, 96%, 97%, 98%, or 99% of a level of expression in a reference sample. In some embodiments, the increase or the decrease in the expression of the one or more genes disclosed herein is an increase or a decrease of at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 relative to a reference sample. In some embodiments, the reference sample is obtained from a subject that does not have the disease or the condition disclosed herein. In some embodiments, the reference sample is obtained from a subject that has the disease or the condition, but does not have the subtype of the disease of the condition described herein. In some embodiments, the therapeutic agent disclosed herein, comprise modulators of miR-181a, miR-92a or miR-124, or any combination thereof. In some embodiments, the therapeutic agents are useful for the treatment of a disease or condition, or symptom of the disease or condition, disclosed herein. For example, the disease or condition comprises a subtype disclosed here, such as, for example, a PAF-CD-mono signature or a CD-mono signature of Crohn's disease. In some embodiments, the disease or the condition is an inflammatory bowel disease (IBD). In some embodiments, the IBD is Crohn's disease (CD) or ulcerative colitis (UC).

In some embodiments, the therapeutic agents comprise a modulator of miR-181a. In some cases, the modulator of miR-181a is an antagonist, partial antagonist, agonist, or partial agonist. In some embodiments, the miR-181a modulator modulates the expression of one or more genes comprising PLAG1, PTPN11, HRAS, BCL2, FOS, DDIT4, BCL2L11, ATM, NOTCH1, MCL1, GATA6, DUSP6, DUSP5, PTPN22, PROX1, KAT2B, CDKN1B, XIAP, MTMR3, SRT1, NLK, KLF6, HPK2, RALA, or a combination thereof.

In certain embodiments, an miR-181a modulator comprises a molecule that upregulates expression of miR-181a. In certain embodiments, an miR-181a modulator comprises a molecule that downregulates or otherwise inhibits miR-181a. In some embodiments, the modulator of miR-181a is an oligomer. In some embodiments, the modulator of miR-181a is a microRNA inhibitor. In some embodiments, the modulator of miR-181a is a microRNA mimic. In a non-limiting exemplary embodiment, the microRNA is microRNA-181a or a precursor thereof, such as a mammalian microRNA-181a. Mammalian microRNA-181a includes human and mouse microRNA-181a.

In some embodiments, the therapeutic agents comprise a modulator of miR-92a. In some cases, the modulator of miR-92a is an antagonist, partial antagonist, agonist, or partial agonist. In some embodiments, the miR-92a modulator modulates the expression of one or more genes comprising BMPR2, PCGF5, TGFBR2, ITGAS, ESR2, CPEB2, OSBPL2, KLF2, KAT2B, HIPK3, MAPRE1, RFFL, OSBPL8, TP63, ARD48, MYLP, or a combination thereof.

In certain embodiments, an miR-92a modulator comprises a molecule that upregulates expression of miR-92a. In certain embodiments, an miR-92a modulator comprises a molecule that downregulates or otherwise inhibits miR-92a. In some embodiments, the modulator of miR-92a is an oligomer. In some embodiments, the modulator of miR-92a is a microRNA inhibitor. In some embodiments, the modulator of miR-92a is a microRNA mimic. In a non-limiting exemplary embodiment, the microRNA is microRNA-92a or a precursor thereof, such as a mammalian microRNA-92a. Mammalian microRNA-92a includes human and mouse microRNA-92a.

In some embodiments, the therapeutic agents comprise a modulator of miR-124. In some cases, the modulator of miR-124 is an antagonist, partial antagonist, agonist, or partial agonist. In some embodiments, the miR-124 modulator modulates the expression of one or more genes comprising CEBPA, BACE1, SMYD3, RELA, AR, MECP2, E2F6, FXN, PEA15, IL6R, SLC116A1, NR3C2, ITGB1, NFKBIZ, CTDSP1, IQGAP1, HMGA1, LAMC1, CDK4, ROCK2, CDK2, RDH10, NR3C1, ELK3, CCL2, AHR, EZH2, MTPN, CDK8, EFNB1, VM, ADPOR2 or a combination thereof.

In certain embodiments, an miR-124 modulator comprises a molecule that upregulates expression of miR-124. In certain embodiments, an miR-124 modulator comprises a molecule that downregulates or otherwise inhibits miR-124. In some embodiments, the modulator of miR-124 is an oligomer. In some embodiments, the modulator of miR-124 is a microRNA inhibitor. In some embodiments, the modulator of miR-124 is a microRNA mimic. In a non-limiting exemplary embodiment, the microRNA is microRNA-124 or a precursor thereof, such as a mammalian microRNA-124. Mammalian microRNA-124 includes human and mouse microRNA-124.

### Pharmaceutical Compositions, Formulations and Methods of Administration

In one aspect, methods of treating a subject, e.g., a subject having a PAF-CD-mono signature, or a CD-mono signature, or any combination thereof, involve administration of a pharmaceutical composition comprising a therapeutic agent described herein, e.g., a modulatory of expression and/or activity of a biomarker disclosed herein, e.g., in Tables 1, 2, or 3, or a modulator of miR-181a, miR-92a or miR-124, or a combination thereof, in therapeutically effective amounts to said subject. In some embodiments, the subject has perianal disease/fistula, stricturing disease, recurrence, or increased immune reactivity to a microbial antigen, or a combination thereof. In some embodiments, a therapeutic agent described herein is used in the preparation of medicaments for treating an inflammatory disease, such as Crohn's Disease.

In certain embodiments, the compositions containing the therapeutic agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a therapeutic agent is administered to a patient suffering from an inflammatory disease such as CD, and optionally comprises a PAF-CD-mono signature or a CD-mono signature.

In prophylactic applications, compositions containing a therapeutic agent described herein are administered to a patient susceptible to or otherwise at risk of a particular disease, disorder or condition, e.g., an inflammatory disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given therapeutic agent that corresponds to such an amount varies depending upon factors such as the particular therapeutic agent, disease condition and its severity, the identity (e.g., weight, sex, age) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a therapeutic agent, the patient is also weaned off (e.g., step-wise decrease in dose) a second treatment regimen.

In one embodiment, the daily dosages appropriate for a therapeutic agent herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended-release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

Disclosed herein are therapeutic agents formulated into pharmaceutical compositions. Pharmaceutical compositions are formulated in a conventional manner using one or more pharmaceutically acceptable inactive ingredients that facilitate processing of the active therapeutic agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a therapeutic agent described herein, and at least one pharmaceutically acceptable inactive ingredient. In some embodiments, the therapeutic agents described herein are administered as pharmaceutical compositions in which the therapeutic agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of a therapeutic agent, with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L 100-55, Eudragit® L 100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the linker.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

The disclosure also provides kits for detecting expression of one or more genes disclosed herein, e.g., in Tables 1, 2, or 3. Exemplary kits include nucleic acids configured for specific hybridization to one or more genes disclosed herein, e.g., in Tables 1, 2, or 3. In some cases a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, a gene disclosed herein, e.g., in Tables 1, 2, or 3. In some embodiments, the kit includes primers and probes for control genes, such as housekeeping genes. In some embodiments, the primers and probes for control genes are used, for example, in ΔCt calculations. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to a biomarker of Tables 1, 2, or 3.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for detecting a particular subtype of IBD or CD in a subject. In some embodiments, the subtype comprises a PAF-CD-mono signature. In some embodiments, the subtype comprises a CD-mono signature. In some embodiments, the subtype is PAF-CD-mono subtype. In some embodiments, the subtype is CD-mono subtype. The system is configured to implement the methods described in this disclosure, including, but not limited to, detecting the presence of a particular CD subtype to determine whether the subject is suitable for treatment with a particular therapy.

In some embodiments, disclosed herein is a system for detecting a IBD subtype in a subject, comprising: (a) a computer processing device, optionally connected to a computer network; and (b) a software module executed by the computer processing device to analyze a target nucleic acid sequence of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system comprises a genotype device, such as a sequencer, polymerase chain reaction (PCR) machine or a genotype array configured for detecting the increase or the decrease in the expression of the one or more genes disclosed herein, e.g., in Table 1 and/or Table 2A or Table 2B. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

The computer-implemented platforms or systems disclosed herein for determining a Crohn's Disease (CD) subtype status in a subject having CD, wherein the status comprises identifying a perianal fistula related CD14+ monocyte subtype (PAF-CD-mono subtype), comprise: (a) one or more processors collectively or individually programmed to implement a method comprising: (i) analyzing gene expression data of the subject to detect a level of expression of one or more genes provided herein, e.g., in Tables 1, 2, or 3 to produce an expression profile of the subject; and (ii) determining the CD subtype status of the subject based upon the expression profile, wherein differential expression of the one or more genes as compared to a reference expression profile indicates that the CD subtype status of the subject comprises a PAF-CD-mono subtype; and (b) a database for storing the gene expression data of the subject and/or the expression profile.

In some embodiments, the subtype is predicted with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the subtype of the disease or condition using the system may be calculated as the percentage of samples identified or classified as having the subtype of the disease or condition that correspond to subjects that truly have the subtype.

In some embodiments, the subtype is predicted with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the subtype of the disease or condition using the system may be calculated as the percentage of samples identified or classified as not having the subtype of the disease or condition that correspond to subjects that truly do not have the subtype.

In some embodiments, the subtype is predicted with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the subtype of the disease or condition using the system disclosed herein may be calculated as the percentage of independent test samples associated with presence of the subtype (e.g., subjects known to have the subtype) that are correctly identified or classified as having the subtype.

In some embodiments, the subtype is predicted with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the subtype of the disease or condition using the system may be calculated as the percentage of independent test samples associated with absence of the subtype (e.g., subjects with negative clinical test results for the subtype) that are correctly identified or classified as not having the subtype.

In some embodiments, the system is configured to identify the presence (e.g., positive test result) or absence (e.g., negative test result) of the subtype with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the algorithm that classifies the samples as having or not having the subtype. The AUC may range from a value of 0 to 1, where an AUC of 0.5 is indicative of a completely random classifier (e.g., a coin flip) and an AUC of 1 is indicative of a perfectly accurate classifier (with sensitivity of 100% and specificity of 100%).

### Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft®.NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

### Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

### Business Methods Utilizing a Computer

The gene expression profiling methods may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g., demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a gene expression profile may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used herein percentage identity or homology may generally refer to percentage of nucleotides that are identical between two or more sequences of DNA or RNA.

As used herein the term “gene” may generally refer to a sequence of nucleotides that comprise a part of a chromosome.

As used herein the term “profile” may generally refer to a compilation of data associated with an individual or a population comprising information specific to that individual or population. In some instances, the information comprises genetic information such as genetic variations or gene expressions specific to that individual or population.

As used herein the term “signature” may generally refer to a single or combined group of genes that are a uniquely characteristic pattern of gene expression associated with a population or subpopulation. In some instances, the characteristic pattern of gene expressions is associated with a phenotype expressed by the population or subpopulation.

As used herein the term “control” or “reference” may generally refer to a group that can be used to in a scientific experiment in which the independent variable cannot influence the outcome.

As used herein, the term “PAF-CD-mono” refers to a subtype of CD14+ monocytes, which has (i) a gene expression profile of a cluster of transcripts/genes that is associated with perianal fistula, or (ii) a property of aggregation with platelets or megakaryocytes which aggregation is associated with perianal fistula, or (iii) both (i) and (ii).

As used herein, the term “PAF-CD-mono subtype,” refers to a subclass of Crohn's disease subjects that have a higher level (e.g., increased number and/or higher percentage) of PAF-CD-mono compared to other CD subjects.

### EXAMPLE

The following illustrative examples are representative of embodiments of the systems and methods described herein and are not meant to be limiting in any way.

### Example 1—Characterizing of Two Functionally Diverse Gene Expression Profiles in Peripheral Monocytes Isolated from Treatment Resistant Crohn's Disease Patients: Diminished Selectin P and CD226 Expression is Associated with Perianal Fistula

### Subjects

Human subjects were recruited through the MIRIAD IBD Biobank at the F. Widjaja Foundation Inflammatory Bowel and Immunobiology Research Institute at Cedars-Sinai Medical Center. Informed consent (approved by the Institutional Review Board at Cedars-Sinai Medical Center) was obtained from all participating subjects. Clinical information was obtained from Crohn's disease (CD) patients (n=48) prior to undergoing surgical resection after which patients were followed prospectively. Subjects without inflammatory bowel disease (IBD) had no known history of IBD and underwent surgery for colon cancer (n=3), colon polyps (n=2), colonic inertia (n=1), diverticulitis (n=2), rectal cancer (n=1), familial adenomatous polyposis (n=1), and rectal polyps (n=1). All CD and non-IBD samples were collected from surgical resections performed by a single provider.

### Methods and Results

CD14+ peripheral monocyte cells were purified from 73 CD patients requiring surgery. Whole RNA was extracted from the CD14+ peripheral monocytes. Libraries for RNA-Seq were prepared with an updated version of the kit (Nugen Universal RNA-seq with NuQuant (part number: 0364 Nugen, Tecan) to generate strand-specific RNA-seq libraries. The workflow consists of poly(A) RNA selection, RNA fragmentation and double-stranded cDNA generation using a mixture of random and oligo(dT) priming, followed by end repair to generate blunt ends, adaptor ligation, strand selection, and PCR amplification to produce the final library. Different index adaptors were used for multiplexing samples in one sequencing lane. RNA sequencing was performed using the Illumina NovaSeq™ 6000 (2×150 output) at 30 million (M) reads/sample from each direction. All libraries were prepared using a single lot or reagents, equipment and processed by same technical staff Samples were processed in two runs with technical and sample duplicates with negligible batch differences. Data quality check was done on Illumina SAV. Demultiplexing was performed with Illumina Bcl2fastq2 v 2.19.1.403 program. STAR aligner software version 2.7.2a was used to align sequenced reads to Human Genome version GRCh38. Reads per gene was quantified using STAR version 2.7.2a with Ensembl GRCh38.98 GTF file. Normalization of reads per gene data was performed with TMM normalization method in Partek Flow software. Samples that had less than 10 million reads mapping to gen exons were eliminated from final normalization. Clean, processed data along with respective meta-data was available in-house. The differential gene expression data from the RNA-Seq is provided in Table 1.

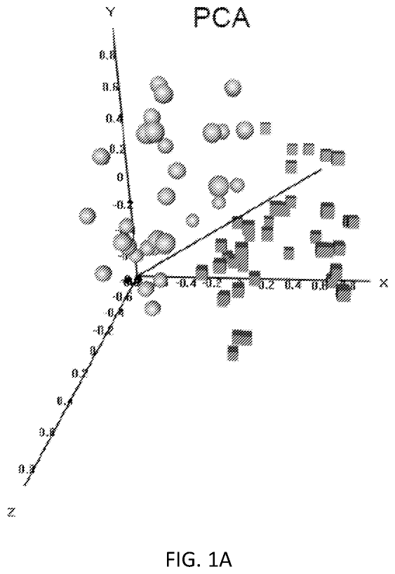

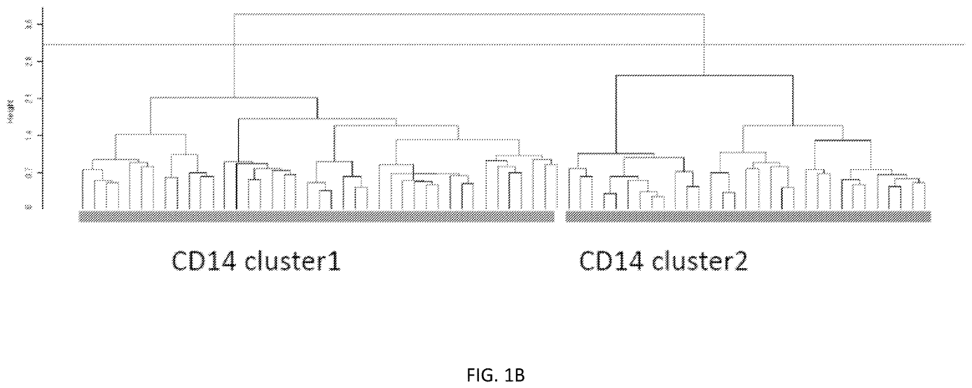

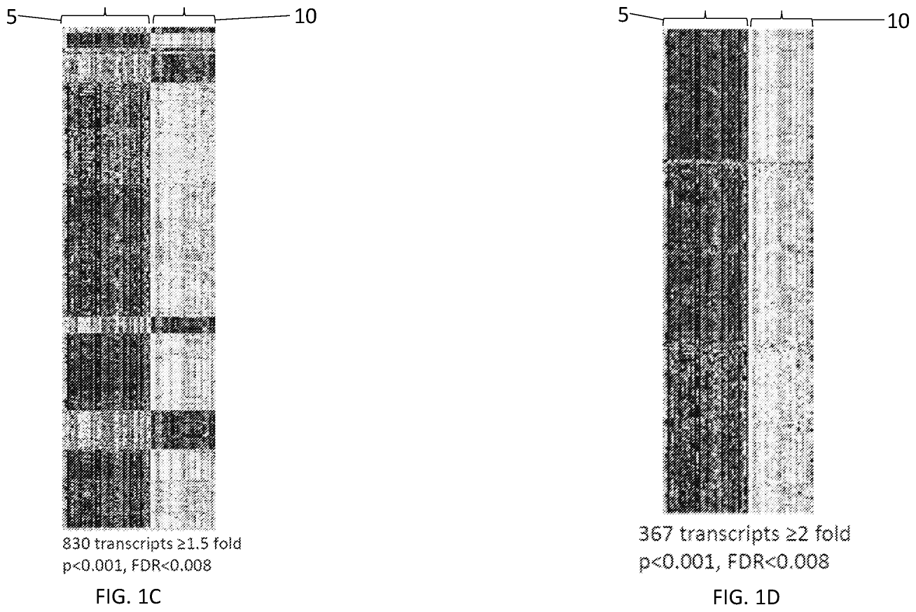

Unsupervised clustering of gene expression stratified severe CD into 2 transcriptomic signatures (FIG. 1B) and identified a distinct monocyte subtype, designated PAF-CD-mono, which was clinically associated with perianal fistula (p=3.7E-04). A principal component analysis (PCA) was performed based on this clustering analysis, which is shown in FIG. 1A. Heat map analyses revealed that 830 transcripts were differentially regulated at least 1.5-fold between the CD14+ cluster 1 subtype (5) and the CD14+ cluster 2 subtype (10) (FIG. 1C) (Table 2) and that 367 transcripts were differentially regulated at least 2-fold between the CD14+ cluster 1 subtype (5) and the CD14+ cluster 2 subtype (10) (FIG. 1D) (Table 1).

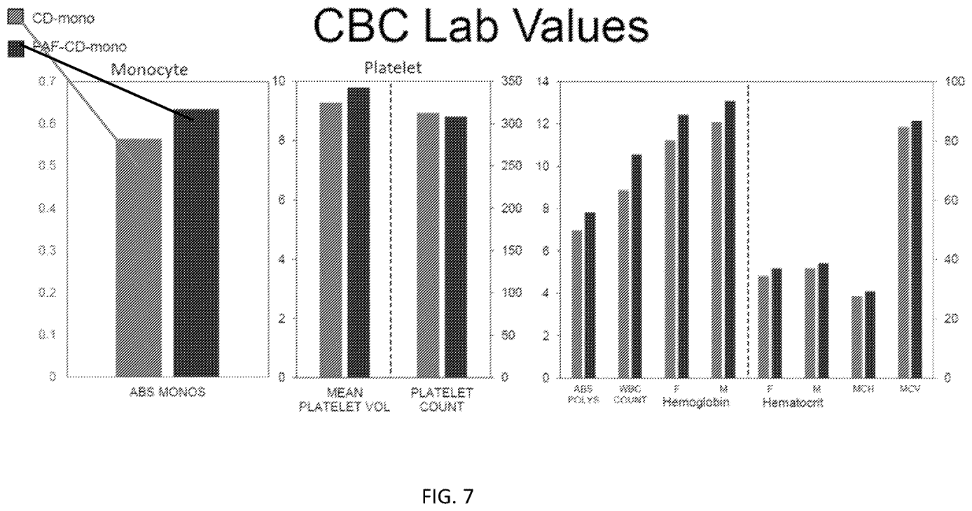

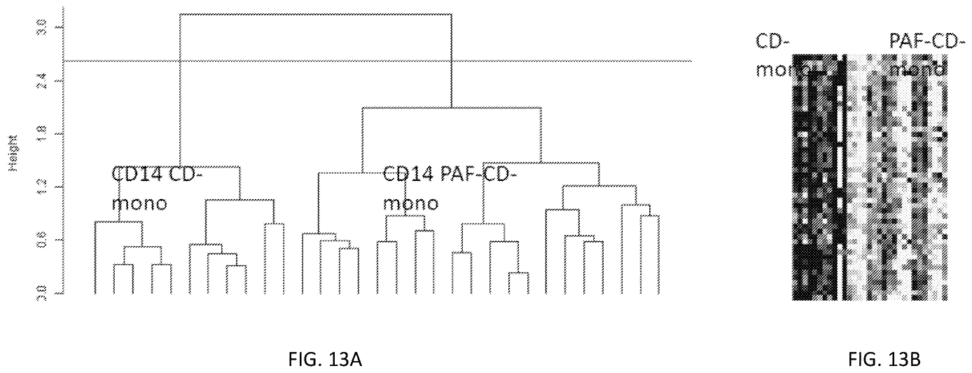

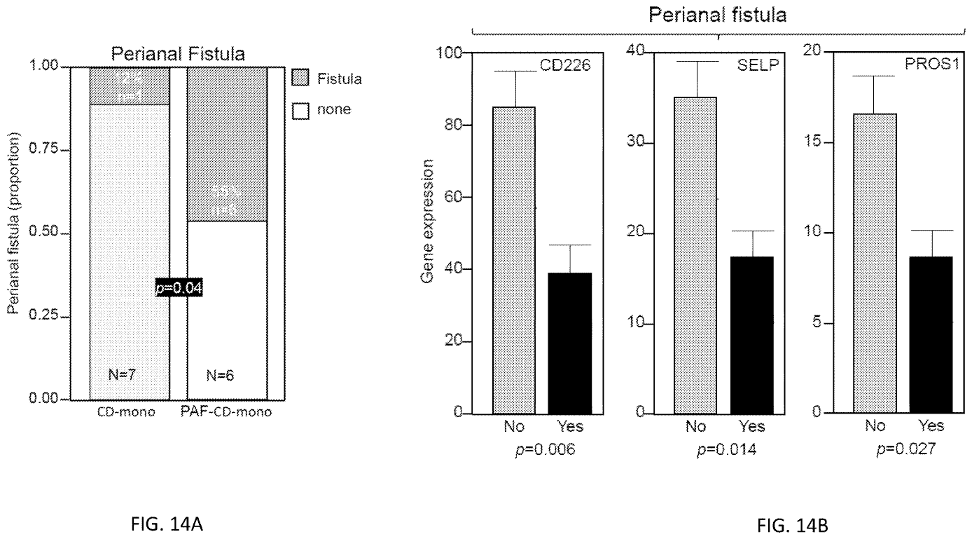

Stratification of CD14⁺ and PAF-CD-mono association with perianal fistula was confirmed in a separate CD patient cohort (see FIGS. 13A-13B and FIGS. 14A-14B). Monocyte subtype signatures were not associated with gender, age, disease location/behavior or therapeutic treatment nor WBC, platelet and monocyte count or CRP lab values at the time of surgery (Table 7 and FIG. 7). Differentially expressed genes (DEG) (regulated at least 1.5-fold) (830, p<0.001, 81% were down-regulated in PAF CD-mono) (Table 2) were significantly enriched in regions flanking GWAS-identified IBD susceptibility variants (12%) (Table 3).

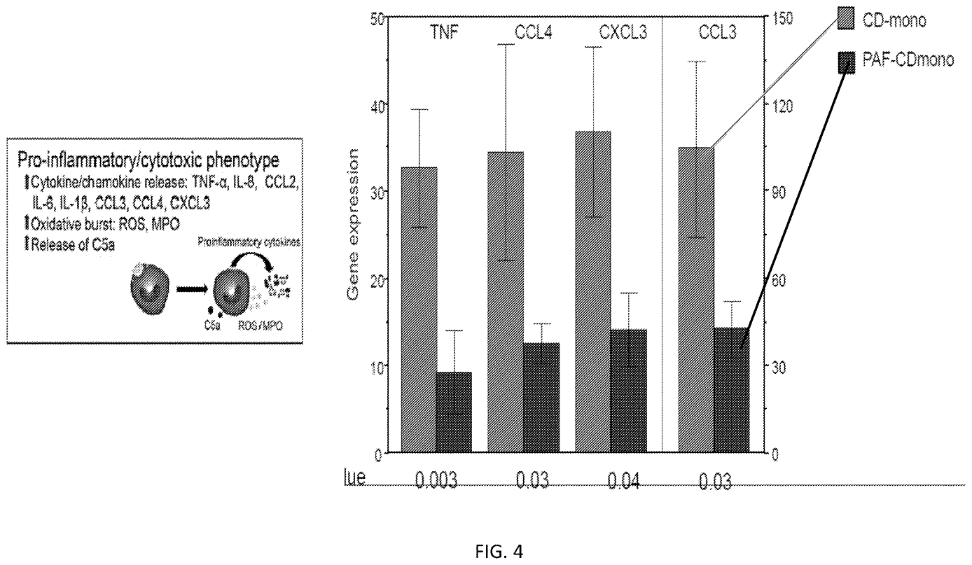

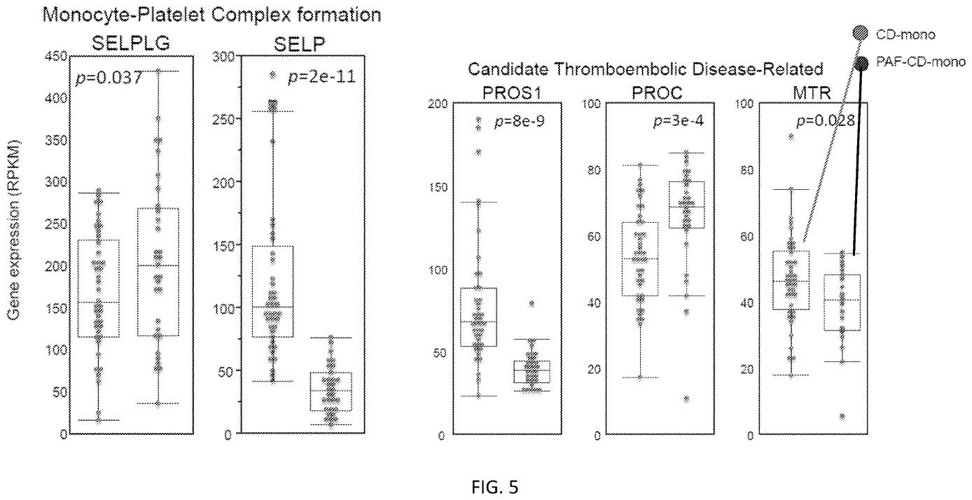

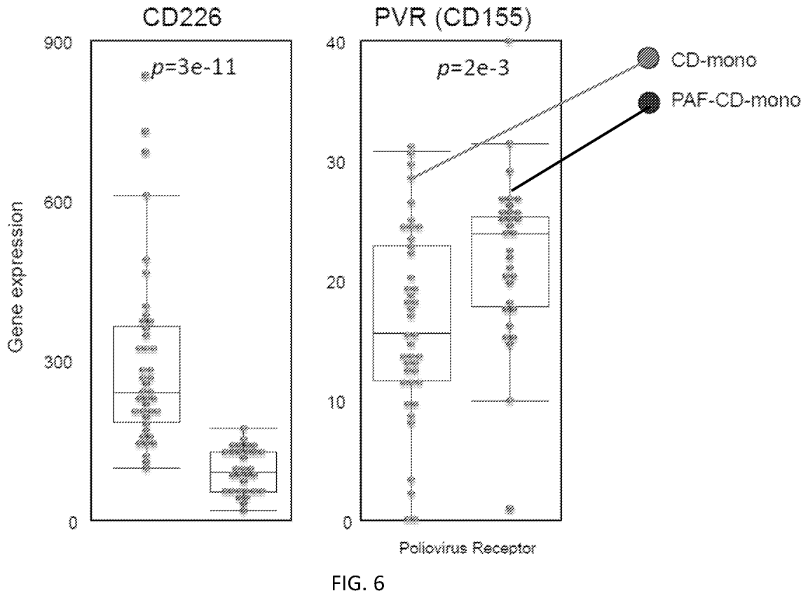

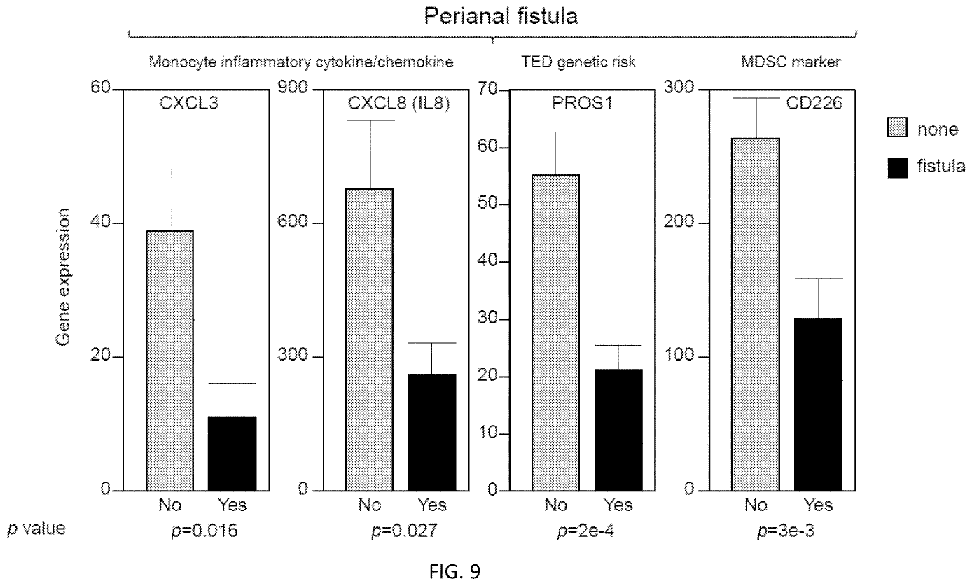

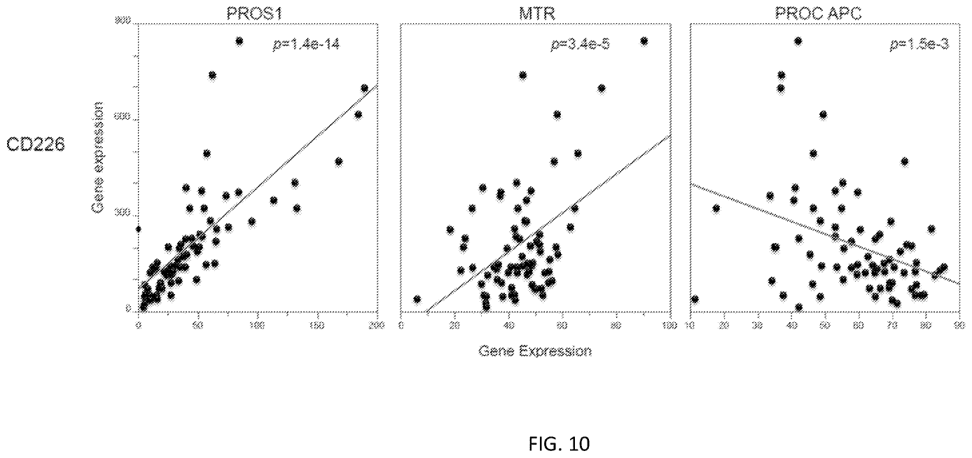

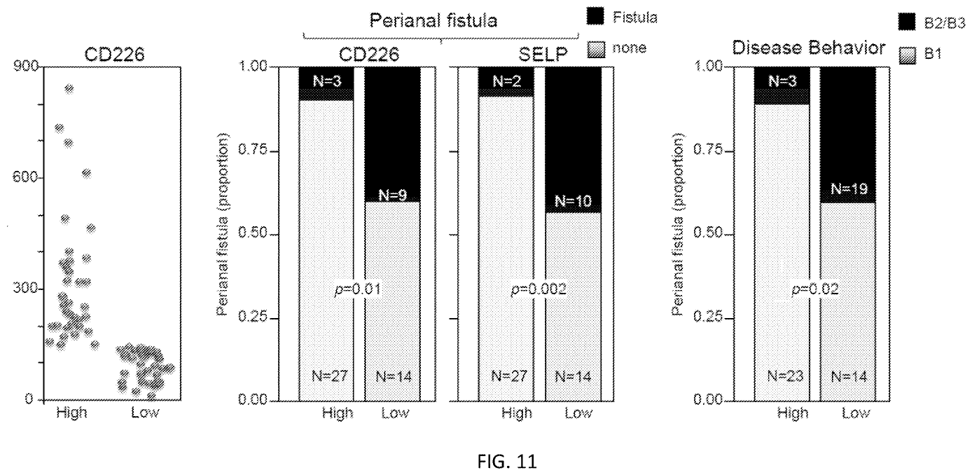

Analysis utilized DGE to show that genes/transcripts involved in platelet-monocyte complex formation (SELPL, SELP), monocyte inflammatory cytokine/chemokine expression (TNF, CCL4, CXCL3, CCL3), IBD candidate thromboembolic disease (TED)-risk genes (PROS1, PROC, MTR) and monocyte inflammatory activation markers (CD1D, CD226-DNAM1) were differentially regulated relative to controls. Each of the transcripts, TNF, CCL4, CXCL3, and CCL3 showed lower expression in the PAF-CD-mono subtype relative to the CD-mono subtype (FIG. 4). Low expression of CD226 (p=0.01) (FIG. 6) and SELP (p=0.002) (FIG. 5) in the PAF-CD-mono subtype, both implicated by GWAS for CD susceptibility, were associated with perianal fistula. In particular, CD226 along with other PAF-CD-mono/monocyte-platelet markers (CXCL3, CXCL8, and PROS1) showed decreased gene expression in perianal fistula (FIG. 9). These findings are consistent with FIG. 10, which shows that CD226 expression is correlated to thromboembolic (TED) associated genes. In addition, low expression of CD226 and SELP was found to be associated with perianal fistula and stricturing (B2)/penetrating (B3) disease behavior (FIG. 11).

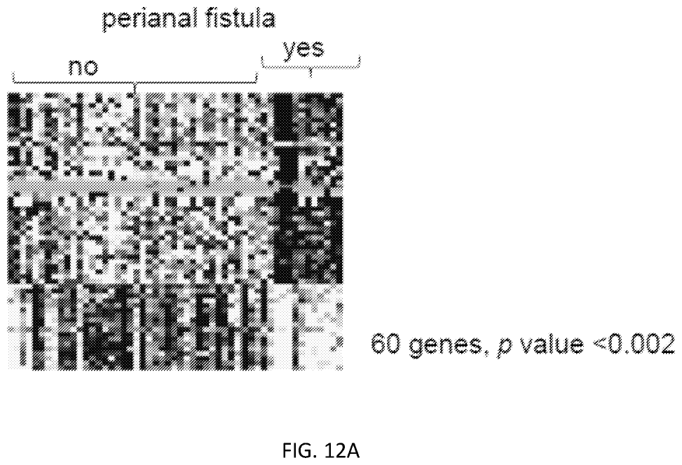

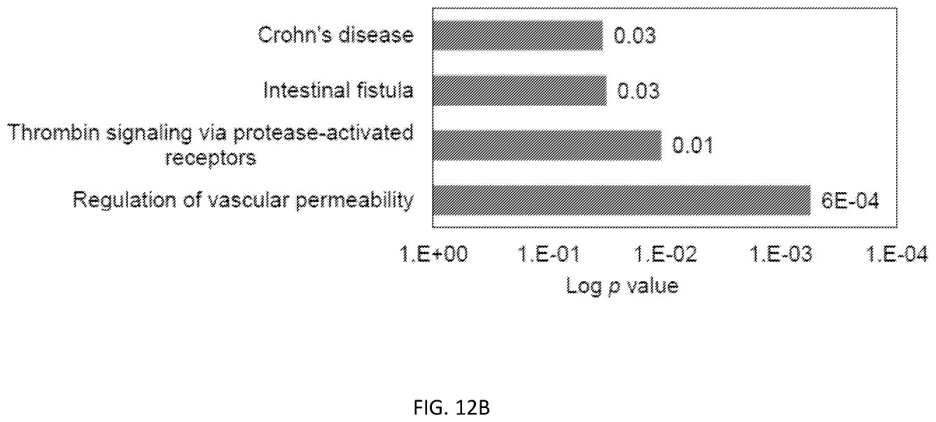

Gene expression was analyzed in monocytes. FIG. 12A shows a heat map of 60 differentially regulated genes expressed between CD patients with (n=10) and without (n=42) perianal fistula. Pathway analysis of the differentially expressed genes showed that differentially regulated genes were highly associated with CD and intestinal fistula (FIG. 12B). These data indicated that differentially expressed genes are associated with the perianal fistula phenotype.

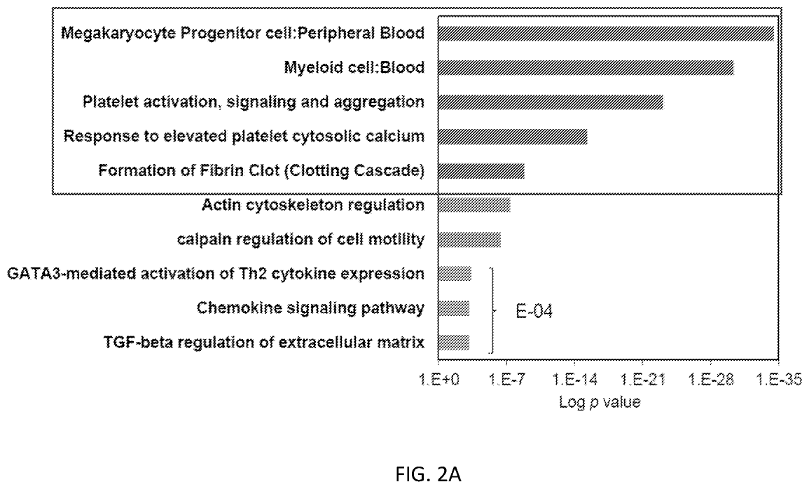

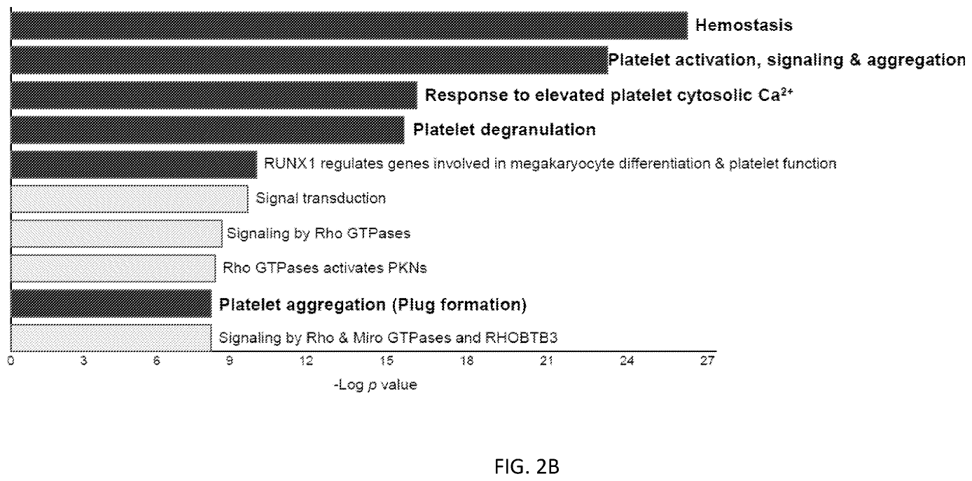

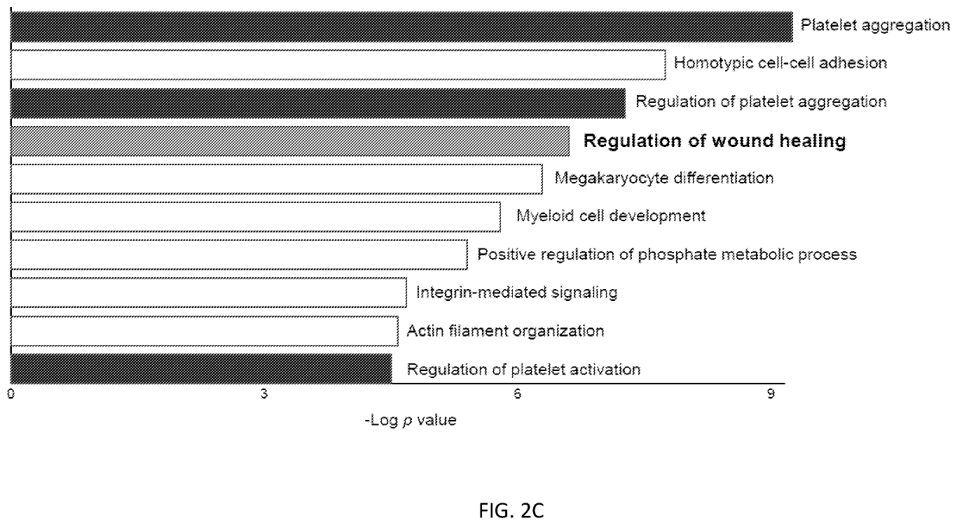

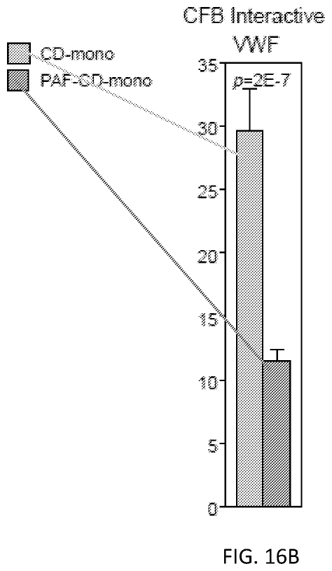

Further experiments were performed to confirm that transcripts clustered into the CD-mono and the PAF-CD-mono subtypes, and that the PAF-CD-mono subtype is associated with perianal fistula. Gene expression was analyzed from 31 CD14+ monocytes, and clustering was based on a differential gene expression filtered list. Of the 31 tested monocytes, 15 were repeat patient samples that were used in the experiments that produced the data in FIGS. 1A and 1B, and 16 were from new patient samples. Among the 15 repeat patient samples, 14 of the 15 classified as they did in FIGS. 1A-1B and FIG. 2A. After removing the repeat samples, of the remaining 16 new patient samples, 15 clustered the same way as shown in FIGS. 13A and 13B. FIG. 13A shows hierarchical clustering of the differentially regulated genes in the 31 tested monocytes and FIG. 13B shows a heatmap of the differentially regulated genes in the 31 tested monocytes. Together, FIGS. 13A and 13B show validation of clustering of separate CD14+ monocytes.

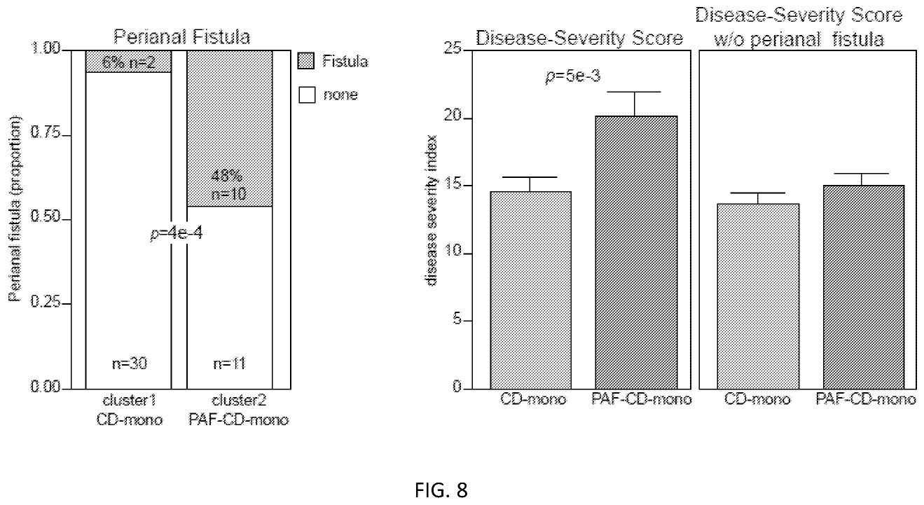

FIGS. 14A and 14B provide data from a validation cohort showing that the PAF-CD-mono subtype is associated perianal fistula, and that certain downregulated genes of the PAF-CD-mono subtype are associated with perianal fistula. The results in FIG. 14A show that perianal fistula is associated with the PAF-CD-mono subtype in a cohort of patients not included in FIG. 8 and FIG. 11. Also, gene expression analysis of this same cohort showed that reduced expression of certain monocyte-platelet markers (CD226, SELP, and PROS1) are associated with perianal fistula (FIG. 14B). Together, FIGS. 14A and 14B show that, in a validation cohort, perianal fistula is associated with the PAF-CD-mono subtype and monocyte-platelet marker expression.

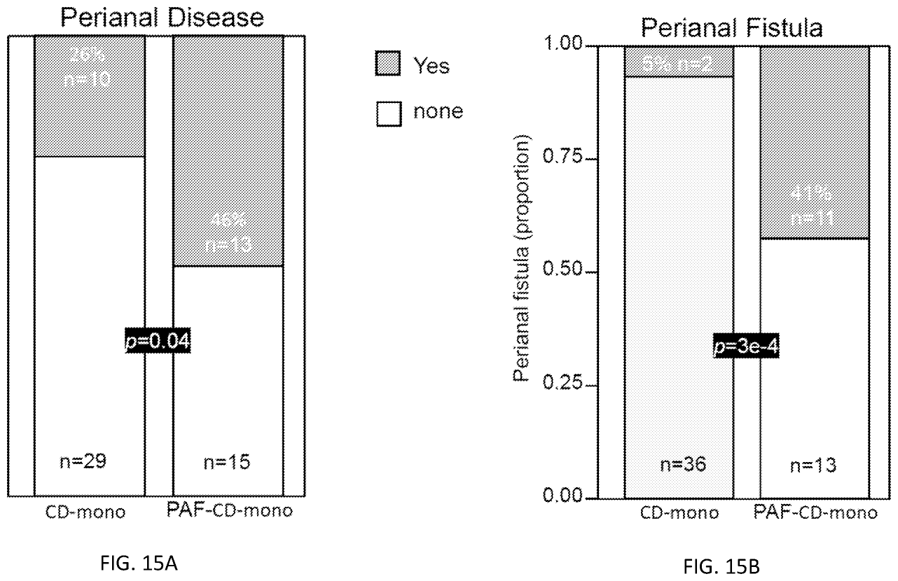

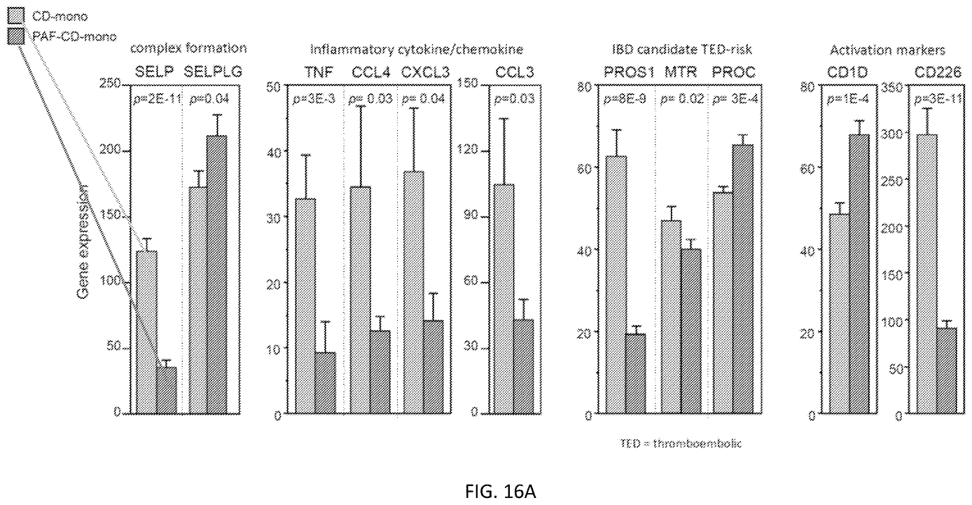

FIGS. 15A and 15B show combined gene expression analysis using two independent RNAseq runs from (i) the cohort tested in FIG. 8 and FIG. 11, as well as (ii) the validation cohort tested in FIGS. 14A and 14B. Together, these data show that perianal disease is associated with the PAF-CD-mono subtype (FIG. 15A) and perianal fistula is associated with the PAF-CD-mono subtype (FIG. 15B).

Pathway analysis (see FIG. 2 and Table 9) provided the focus to further define perianal-fistula biomarkers. Experiments were run to examine differential expressed genes implicated in platelet-monocyte complex formation (SELPL, SELP), monocyte inflammatory cytokine/chemokine expression (TNF, CCL4, CXCL3, CCL3), IBD candidate thromboembolic disease (TED)-risk genes (PROS1, PROC, MTR) and monocyte activation/inflammatory markers (CD1D and CD226). As shown in FIG. 16A, SELP, TNF, CCL4, CXCL3, CCL3, PROS1, MTR, and CD226 each had lower expression levels in the PAF-CD-mono subtype relative to the CD-mono subtype. SELPLG, PROC, and CD1D each had higher expression levels in the PAF-CD-mono subtype relative to the CD-mono subtype. As shown in FIG. 16B, VWF had a lower expression level in the PAF-CD-mono subtype relative to the CD-mono subtype.

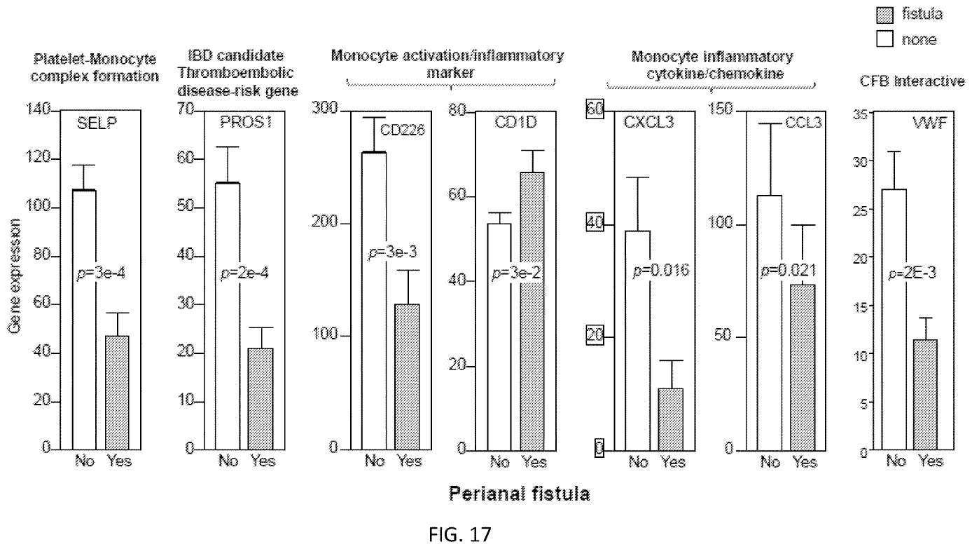

Additional experiments showed that perianal fistula is associated with dysregulated gene expression in gene pathways associated with certain platetelet-mediated monocyte activation pathways. As shown in FIG. 17, SELP (a gene associated with platelet-monocyte complex formation), PROS1 (an IBD candidate thromboembolic disease-risk gene), CD226 and CD1D (monocyte activation/inflammatory markers), CXCL3 (a monocyte inflammatory cytokine), CCL3 (a monocyte inflammatory cytokine), and VWF (a blood glycoprotein that promotes hemostasis, including platelet adhesion) were each differentially regulated in perianal fistula relative to the control.

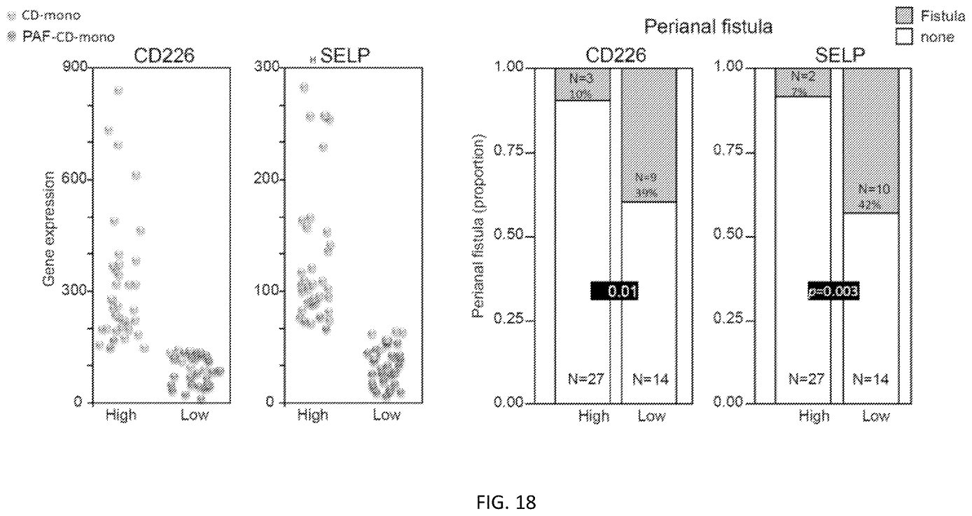

Experiments were also performed to examine perianal risk-stratification based on gene expression levels of CD226 and SELP. Patients were stratified based on relative high or low levels of expression of CD226 and SELP irrespective of subset classification in CD-mono or PAF-CD-mono. As shown in FIG. 18, increased risk of perianal fistula is associated with low expression of both CD226 and SELP.

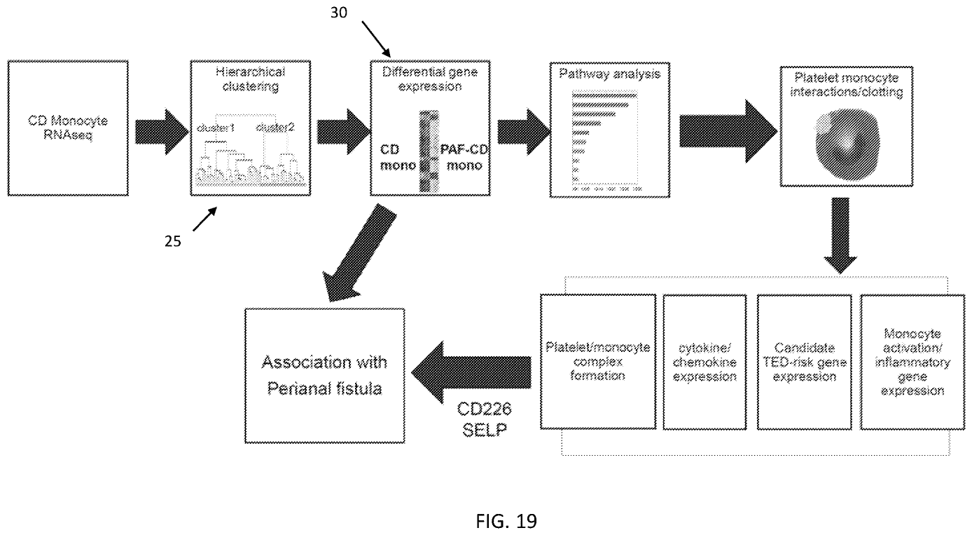

FIG. 19 provides an embodiment of a schematic analysis that can be used for identifying CD14+ subtypes. Specifically, FIG. 19 shows that (i) using hierarchical cluster analysis to stratify the genes into different subpopulations (25) and (ii) using differential gene expression comparing CD-mono and PAF-CD-mono subtypes (30) are effective in showing genes that are differentially expressed in CD14+ subtypes.

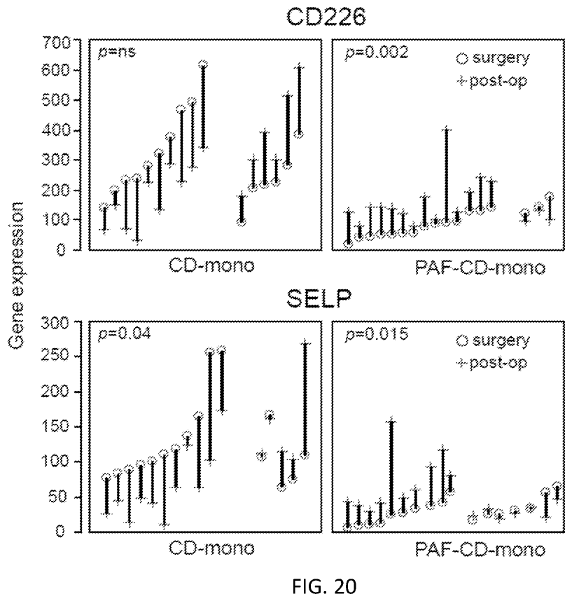

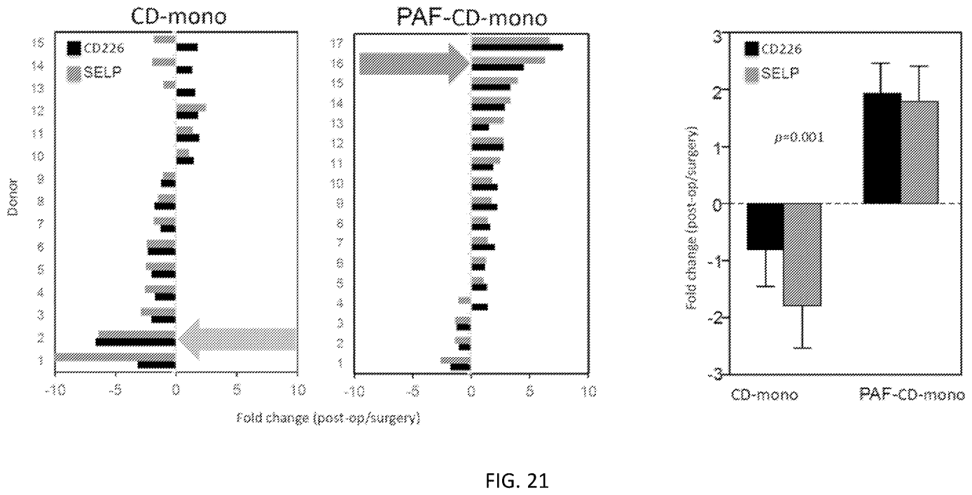

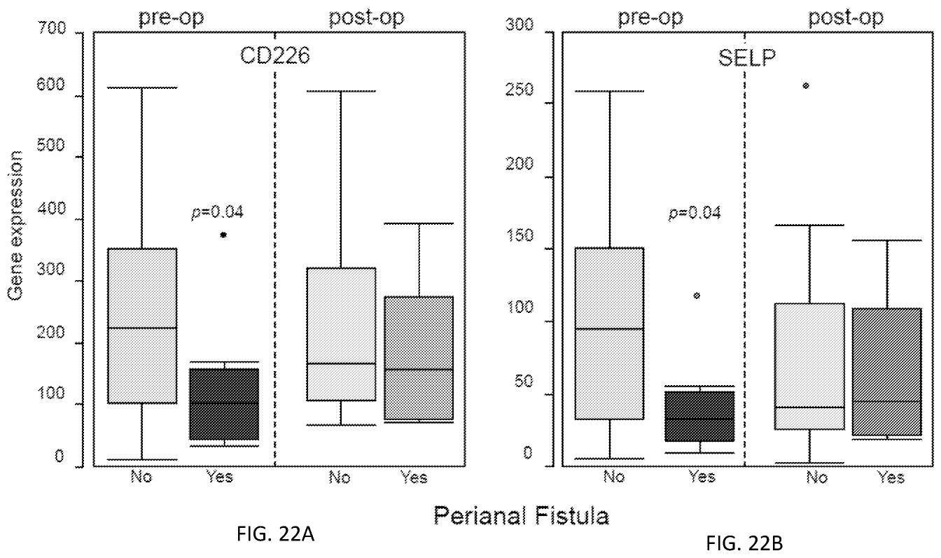

Additional experiments demonstrated that markers of the different CD14+ subtypes can change post-peratively. FIG. 20, and Table 8 show that the monocyte-platelet markers CD226 and SELP change post-operatively in the CD-mono and the PAF-CD-mono subtypes. FIG. 21 shows data from 33 patients, of which 16 were the CD-mono subtype and 17 were the PAF-CD-mono subype. CD226 and SELP expression levels were tested before and after surgery. Samples from the patients were taken between 4 and 13 months after surgery. FIG. 21 shows CD226 and SELP gene expression is altered following surgery. In addition, post-operatively, the association of CD226 and SELP with perianal fistula is lost (FIGS. 22A and 22B).

### Crohn's Disease Severity Score

A pre-operative Crohn's disease severity score was calculated based on a modified disease severity weighted index previously described. (Siegel CA, Whitman C B, Spiegel B M, et al. Development of an index to define overall disease severity in IBD. Gut. 2016.) The attributes included fistula, perianal abscess, steroid use, biologics/immunologics use, stricture and disease extent. Patients who had previous resections were assigned a weighted score of 3 and a score of 0 indicated no prior resection. All laboratory procedures were performed by staff blinded to the patient clinical phenotype. FIG. 8 shows that the PAF-CD-mono subset is associated with perianal fistula and a higher disease-severity score.

### Statistical Analysis

RNA-Seq data analysis and data mining were performed using the BRB array tools (brb.nci.nih.gov/BRB-ArrayTools, version 4.6.1) and R-program (www.r-project.org). Class prediction analysis used Bayesian covariate predictor, diagonal linear discriminant analysis, k-nearest neighbor (using k=1 and 3), nearest centroid, support vector machines and non-negative matrix factorization multivariate classification methods, based upon a minimum p value of 0.001. A 0.632+ bootstrap cross-validation randomly re-sampling method was used to compute mis-classification rate. False Discovery Rate to control for multiple hypothesis testing was calculated by Benjamini and Hochberg method. Cluster analysis was performed using BRB array tools and Cluster 3.0 with Java Treeview. Tests for statistical significance were determined using JMP Statistical Software (Cary, NC). Data were assessed for normality by the Shapiro-Wilk test. If data were normal, a 2-tailed, unpaired Student's t test was used. For non-normal data, Wilcoxon or Kolmogorov-Smirnov test was used to calculate p values. A univariate model was fitted with CD subtypes for demographic and clinical data.

### Pathway Analysis and Co-Expression Analysis

Enrichr (http://amp.pharm.mssm.edu/Enrichr/) or BRB array tools GO, and KEGG pathway enrichment analysis were used to analyze pathway enrichment of differentially expressed genes. Pathway analysis strongly associated CD14 differential gene expression with megakaryocyte progenitor cell markers, monocyte-platelet aggregation/activation/signaling and the clotting cascade (p=E-29 to -07) (FIG. 2A and Table 9). FIG. 2B provides Reactome pathway analysis showing that CD-mono differential gene expression is associated with platelet activation and clotting pathways. FIG. 2C provides GO biological process pathway analysis showing that CD-mono differential gene expression is associated with platelet activation, adhesion, and wound healing pathways.

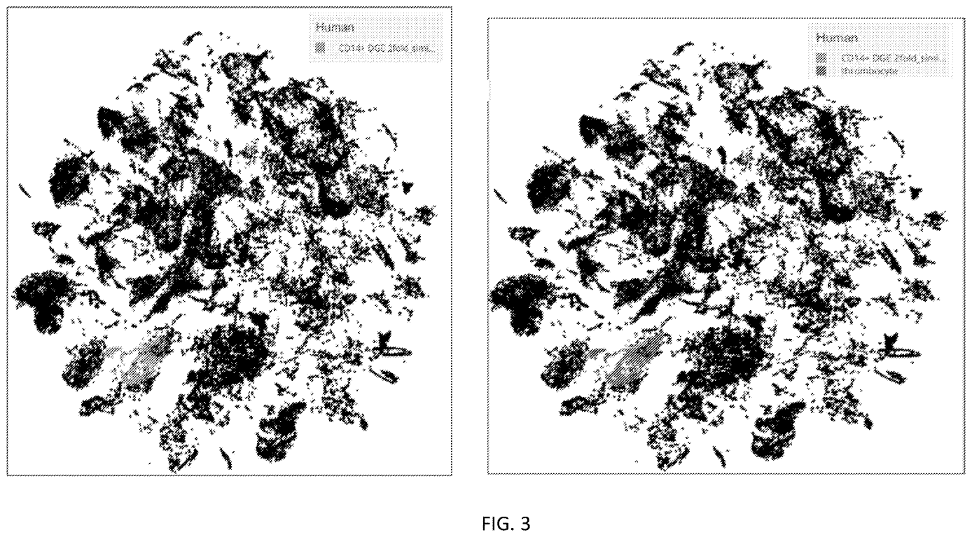

ARCHS4 generated t-SNe plots showed that the gene signature from the differentially upregulated gene panel in PAF-CD-mono vs. CD-mono overlaps with similar coexpression from thrombocytes (FIG. 3).

### Circulating CD226 Protein Levels

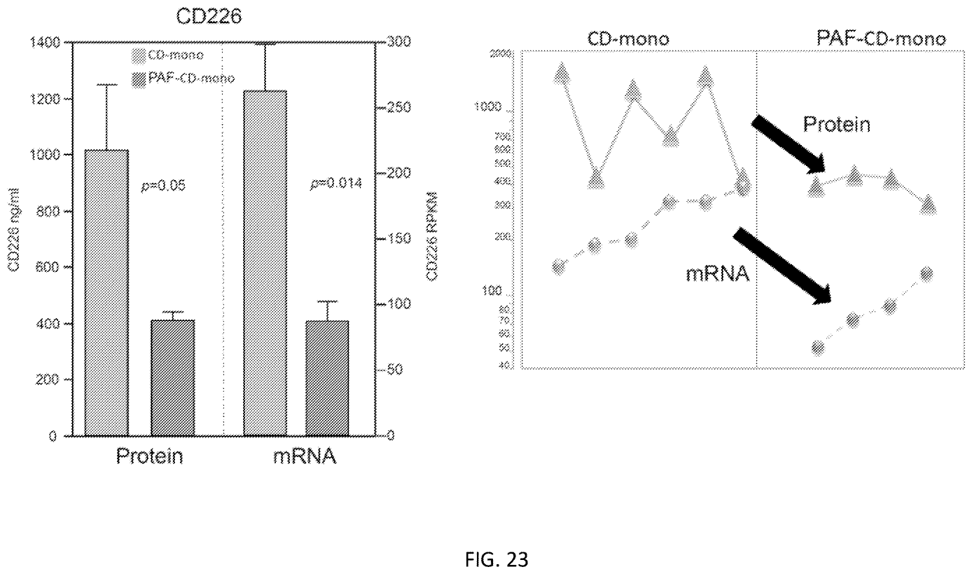

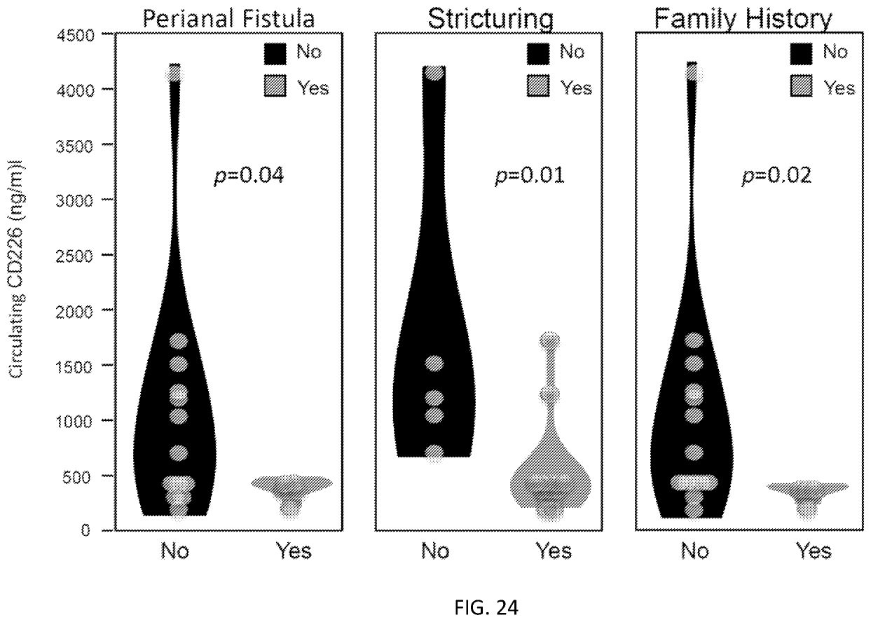

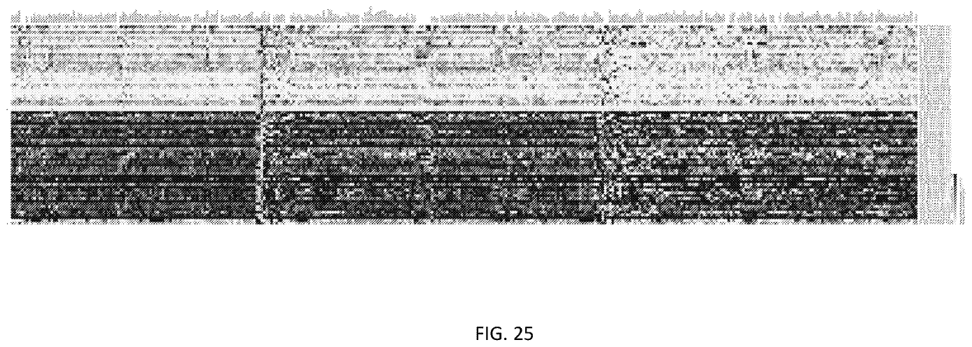

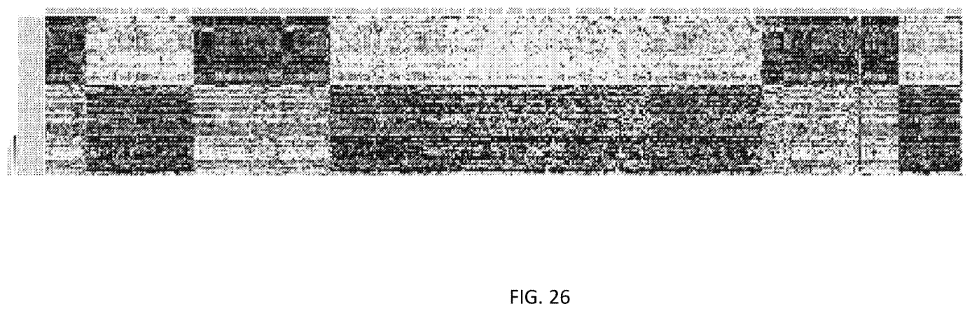

Plasma was isolated from blood of CD patients undergoing surgery and circulating CD226 protein levels measured by ELISA (R&D Systems, Inc., Minneapolis, MN). As shown in FIG. 23, the level of circulating CD226 is plasma at the time of surgery is decreased in the PAF-CD-mono subtype and this level of circulated CD226 is consistent with CD226 mRNA levels. Moreover, as shown in FIG. 24, low levels of circulating CD226 protein are associated with perianal fistula, stricturing, and family history.

TABLE 7

| Patient demographics | CD-mono | | PAF-CD-mono | |
|---|---|---|---|---|
| Gender Female (%) | 47 | | 38 | |
| Age at diagnosis (median | 23 | (16-39) | 23 | (15-30) |
| and IQR), yr. | | | | |
| Disease duration (median | 8 | (2-14) | 9 | (4-13) |
| and IQR), yrs. | | | | |
| Age at surgery (median | 34 | (25-53) | 32 | (24-42) |
| and IQR), yr. | | | | |
| elevated CRP at surgery (%) | 58 | | 67 | |
| Steroids (%) | 73 | | 85 | |
| 5-ASA (%) | 78 | | 67 | |
| anti-TNF (%) | 57 | | 63 | |
| Resected bowel length | 32 | (21-57) | 32 | (21-49) |
| (median and IQR), cm. | | | | |
| Disease location | | | | |
| L1, % (n) | 9 | (4) | 3 | (1) |
| L2, % (n) | 14 | (6) | 3 | (1) |
| L3, % (n) | 77 | (34) | 93 | (28) |
| Hematology lab values | | | | |
| WBC (10⁹/L) | 8 | (6-12) | 10 | (7-15) |
| MCV fl | 86 | (78-90) | 87 | (82-91) |
| MCH pg | 29 | (26-31) | 30 | (27-32) |
| Platelet count (10⁹/L) | 303 | (214-381) | 262 | (207-397) |
| Mean platelet volume fl | 9.4 | (8.5-10.2) | 9.9 | (8.6-11.2) |
| ABS Monocytes (10⁹/L) | 0.55 | (0.33-0.7) | 0.6 | (0.43-0.87) |

TABLE 8

| | |
|---|---|
| CD-Mono | PAF-CD-mono |

| Gene | p value | direction | p value | direction |
|---|---|---|---|---|
| CCL3 | 0.029 | ↓ | | |
| CD226 | | | 0.002 | ↑ |
| CXCL3 | 0.021 | ↓ | | |
| PROS1 | | | | |
| SELP | 0.044 | ↓ | 0.015 | ↑ |
| SELPLG | 0.044 | ↓ | | |
| TNF | | | 0.006 | ↑ |

TABLE 9
DGE Pathways

| Term | P-value |
|---|---|
| Megakaryocyte Progenitor cell: Peripheral Blood | 2.55E−29 |
| Homeostasis | 8.06E−25 |
| Platelet Activation, Signaling and Aggregation | 5.37E−22 |
| Response To Elevated Platelet Cytosolic Ca2+ | 1.05E−14 |
| Formation Of Fibrin Clot (Clotting Cascade) | 1.87E−07 |
| Genes Down-Regulated in Macrophages Vs Monocytes | 8.61E−06 |
| Serotonin transporter activity | 0.000769 |
| Chemokine signaling pathway | 0.000918 |

### Example 2—CD226 rs783361 is Associated with Decreased Expression of CD226 and Perianal Fistula

CD226 rs763361 is a missense mutation that leads to an amino acid substitution (Gly307Ser). The rs763361 T variant is associated with risk for coeliac disease, type 1 diabetes, multiple sclerosis (MS), rheumatoid arthritis (RA) and Graves disease, as well as decreased monocyte mRNA expression and cell activation. Moreover, the CD226 risk isoform has increased activity and reduced Treg suppressive capacity.

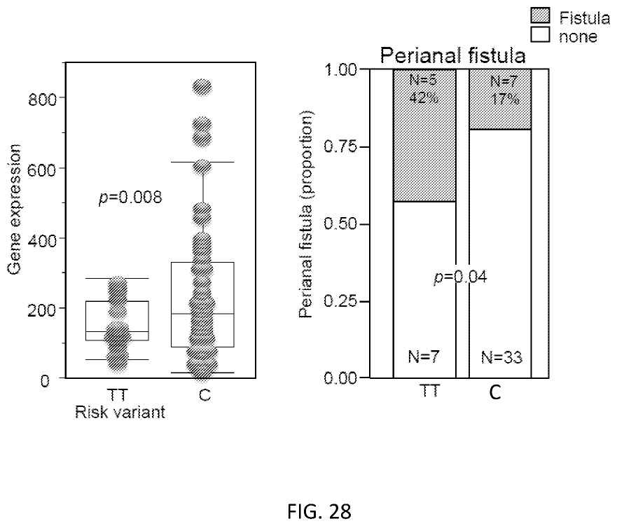

Experiments were performed to evaluate the effect of rs763361 on CD226 gene expression and risk of perianal fistula. The subjects and methods were essentially as described in Example 1. As shown in FIG. 28, left panel, subjects whose genotypes comprise the rs763361 T variant (e.g., subjects who were homozygous for the T variant) had decreased gene expression of CD226 compared to subjects whose genotypes comprise the C allele at rs763361. Additionally, subjects whose genotypes comprise the rs763361 T variant (e.g., subjects who were homozygous for the T variant) had an increased risk of perianal fistula (right panel).

These results indicate that the presence of the rs763361 T variant in a subject's genome can be used, e.g., for determining a subject's Crohn's Disease (CD) subtype status and/or risk of perianal fistula, as well as for patient selection for CD therapies.

### Example 3—Innate Immune Response, Adhesion, and Toll-Like Receptor (TLR) Markers are Elevated in PAF-CD-Mono Subtype Subjects

Experiments were performed to evaluate the expression of innate immune response, adhesion, and TLR markers in CD-mono subtype subjects compared to PAF-CD-mono subjects. The subjects and methods were essentially as described in Example 1.

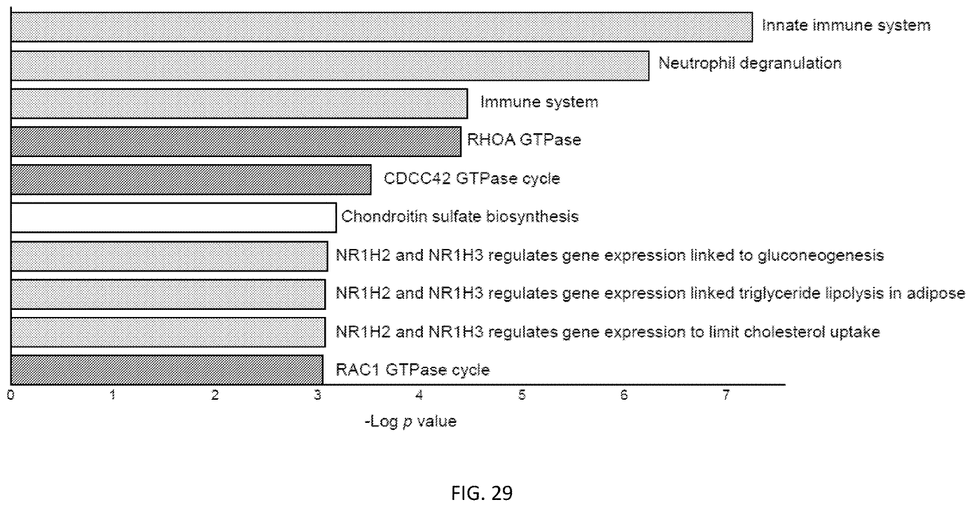

FIG. 29 shows results from Reactome pathway analysis showing that PAF-CD-mono upregulated differential gene expression is associated with innate immune regulation.

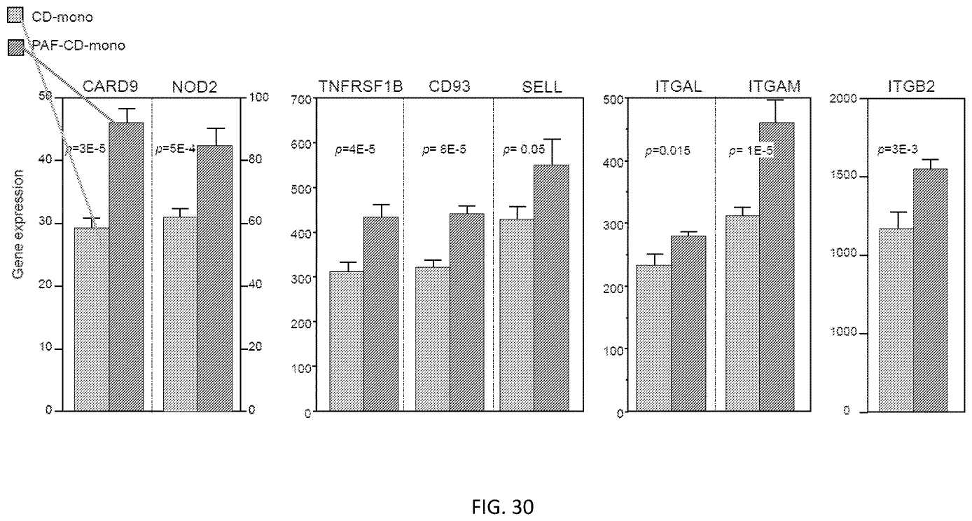

FIG. 30 shows results showing that innate immune response and adhesion markers including CARD9, NOD2, TNFRSF1B, CD93, SELL, ITGAL, ITGAM, and IGTB2 were elevated in PAF-CD-mono as compared to CD-mono.

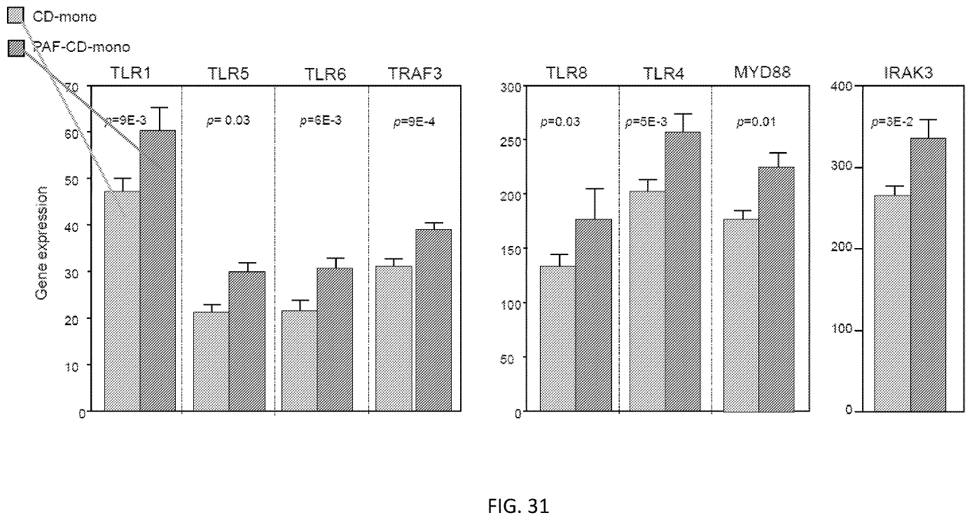

FIG. 31 provides results showing that expression of Toll-like receptor (TLR) signaling molecules including TLR1, TLR5, TRLR6, TRAF3, TLR8, TLR4, MYD88, and IRAK3 are elevated in PAF-CD-mono as compared to CD-mono.

These results indicate that the expression level of innate immune response and adhesion markers, including CARD9, NOD2, TNFRSF1B, CD93, SELL, ITGAL, ITGAM, and IGTB2, can be used e.g., for determining a subject's CD subtype status and/or risk of perianal fistula, as well as for patient selection for CD therapies. These results further indicate that the expression level of TLR signaling molecules, including TLR1, TLR5, TRLR6, TRAF3, TLR8, TLR4, MYD88, and IRAK3, can be used e.g., for determining a subject's CD subtype status and/or risk of perianal fistula, as well as for patient selection for CD therapies.

What is claimed is:

1. A method of treating a Crohn's Disease (CD) subtype status, wherein the status comprises a perianal fistula related CD14+ monocyte subtype (PAF-CD-mono subtype), the method comprising:
detecting expression of any one or more of the genes CD226, SELP, TNF, CCL4, CXCL3, CCL3, SELPLG, PROS1, PROC, MTR, CD155, and CXCL8 to obtain an expression profile;
determining the CD subtype status based on the expression profile,
wherein differential expression of the any one or more genes as compared to a reference expression profile indicates status of PAF-CD-mono subtype; and
administering a therapeutically effective amount of a therapeutic agent for treating CD.

2. A method of treating a Crohn's Disease (CD) subtype status, wherein the status comprises a perianal fistula related CD14+ monocyte subtype (PAF-CD-mono subtype), the method comprising:
detecting expression of any one or more the genes from Tables 1, 2, and 3 to obtain an expression profile;
determining the CD subtype status based on the expression profile,
wherein differential expression in the any one or more genes as compared to a reference expression profile indicates status of PAF-CD-mono subtype; and
administering a therapeutically effective amount of a therapeutic agent for treating CD.

3. A method of treating a Crohn's Disease (CD) subtype status, wherein the status comprises a perianal fistula related CD14+ monocyte subtype (PAF-CD-mono subtype), the method comprising:
detecting expression of any one or more the genes of the cell type cluster PAF-CD-mono to obtain an expression profile;
determining the CD subtype status based on the expression profile,
wherein differential expression in the any one or more genes as compared to a reference expression profile indicates status of PAF-CD-mono subtype; and
administering a therapeutically effective amount of a therapeutic agent for treating CD.

4. A method of treating a Crohn's Disease (CD) subtype status, wherein the status comprises a perianal fistula related CD14+ monocyte subtype (PAF-CD-mono subtype), the method comprising:
detecting expression of any one or more of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF to obtain an expression profile;
determining the CD subtype status based on the expression profile,
wherein differential expression of the any one or more genes as compared to a reference expression profile indicates status of PAF-CD-mono subtype; and
administering a therapeutically effective amount of a therapeutic agent for treating CD.

5. The method of any of claims 1-4, wherein differential expression in the any one or more genes comprises reduced expression in any one or more of the genes.

6. The method of any of claims 1-4, wherein differential expression in the any one or more genes comprises increased expression in any one or more of the genes.

7. The method of any of claims 1-4, wherein the therapeutic agent comprises an anti-TL1A antibody.

8. The method of any of claims 1-4, wherein the therapeutic agent comprises a steroid.

9. The method of any of claims 1-4, wherein the therapeutic agent comprises an immunosuppressant.

10. The method of claim 1, wherein the any one or more genes comprise any one or more of SELP and CD226.

11. The method of claim 2, wherein any one or more of the genes from Tables 1, 2, and 3, comprises genes associated with any one or more of (i) platelet-monocyte interaction and clotting pathways, (ii) monocyte mediated inflammatory cytokine/chemokine expression, (iii) candidate TED-risk gene expression, and (iv) MDSC surface marker expression.

12. The method of claim 11, wherein the genes associated with candidate TED-risk gene expression comprise any one or more of PROS1, MTR, and PROC APC.

13. The method of claim 2, wherein the any one or more the genes from Tables 1, 2, and 3, comprises genes associated with any one or more of perianal fistula and perianal disease.

14. The method of claim 13, wherein the genes associated with any one or more of perianal fistula and perianal disease comprise any one or more of CD226, SELP, and PROS1.

15. The method of any of claims 1-14, wherein differential expression of the any one or more of the genes is due to aggregation of CD14+ monocytes with (i) platelets, (ii) megakaryocytes, or (iii) both platelets and megakaryocytes.

16. A method of treating a Crohn's Disease (CD) subtype status in a subject, wherein the status comprises a perianal fistula related CD14+ monocyte subtype (PAF-CD-mono subtype), the method comprising administering a therapeutically effective amount of a therapeutic agent for treating CD, provided that one or more polymorphisms comprising rs763361 or a proxy polymorphism in linkage disequilibrium therewith as determined with an r² of at least 0.85, or a combination thereof, are detected in a biological sample obtained from the subject.

17. A method of treating moderate to severe Crohn's Disease (CD) in a subject, the method comprising:
administering a therapeutically effective amount of a therapeutic agent for treatment of the CD, provided the subject is determined to have a perianal fistula related CD14+ monocyte subtype (PAF-CD-mono subtype) based, at least in part, on (i) differential expression of one or more genes from Tables 1, 2, and 3 in a biological sample obtained from the subject, relative to a reference expression profile; (ii) differential expression of one or more of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF in a biological sample obtained from the subject, relative to a reference expression profile;
and/or (iii) the presence of one or more polymorphisms comprising rs763361 or a proxy polymorphism in linkage disequilibrium therewith as determined with an r² of at least 0.85, or a combination thereof, in a biological sample obtained from the subject.

18. The method of claim 17, wherein the one or more genes from Tables 1, 2, or 3 comprise genes of the cell type cluster PAF-CD-mono.

19. The method of claim 18, wherein the one or more genes from Tables 1, 2, or 3 comprise any one or more of CD226, SELP, TNF, CCL4, CXCL3, CCL3, SELPLG, PROS1, PROC, MTR, CD155, and CXCL8.

20. The method of claim 18, wherein the one or more genes from Tables 1, 2, or 3 comprises any one or more of SELP and CD226.

21. The method of any of claims 17-20, wherein the PAF-CD-mono subtype is associated with perianal disease or perianal fistula.

22. The method of any of claims 17-20, wherein the therapeutic agent comprises an anti-TL1A antibody.

23. The method of any of claims 17-20, wherein the therapeutic agent comprises a steroid.

24. The method of any of claims 17-20, wherein the therapeutic agent comprises an immunosuppressant.

25. A method of determining a Crohn's Disease (CD) subtype status, wherein the status comprises a perianal fistula related CD14+ monocyte subtype (PAF-CD-mono subtype), the method comprising:
detecting expression of any one or more of the genes CD226, SELP, TNF, CCL4, CXCL3, CCL3, SELPLG, PROS1, PROC, MTR, CD155, and CXCL8 to obtain an expression profile; and
determining the CD subtype status based on the expression profile,
wherein differential expression in the any one or more genes as compared to a reference expression profile indicates status of PAF-CD-mono subtype.

26. A method of determining a Crohn's Disease (CD) subtype status, wherein the status comprises a perianal fistula related CD14+ monocyte subtype (PAF-CD-mono subtype), the method comprising:
detecting expression of any one or more of the genes from Tables 1, 2, and 3 to obtain an expression profile; and
determining the CD subtype status based on the expression profile,
wherein differential expression in the any one or more genes as compared to a reference expression profile indicates status of PAF-CD-mono subtype.

27. A method of determining a Crohn's Disease (CD) subtype status, wherein the status comprises a perianal fistula related CD14+ monocyte subtype (PAF-CD-mono subtype), the method comprising:
detecting expression of any one or more the genes of the cell cluster type PAF-CD-mono to obtain an expression profile; and
determining the CD subtype status based on the expression profile,
wherein differential expression in the any one or more genes as compared to a reference expression profile indicates status of PAF-CD-mono subtype.

28. A method of determining a Crohn's Disease (CD) subtype status, wherein the status comprises a perianal fistula related CD14+ monocyte subtype (PAF-CD-mono subtype), the method comprising:
detecting expression of any one or more of the genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF to obtain an expression profile; and
determining the CD subtype status based on the expression profile,
wherein differential expression in the any one or more genes as compared to a reference expression profile indicates status of PAF-CD-mono subtype.

29. The method of any of claims 25-28, wherein differential expression in the any one or more of the genes comprises reduced expression in any one or more of the genes.

30. The method of any of claims 25-28, wherein differential expression in the any one or more of the genes comprises increased expression in any one or more of the genes.

31. The method of any of claims 25-28, 29, and 30, wherein any one or more of the genes comprises any one or more of SELP and CD226.

32. The method of claim 31, wherein any one or more of the genes from Tables 1, 2, and 3, comprise genes associated with any one or more of (i) platelet-monocyte interaction and clotting pathways, (ii) monocyte mediated inflammatory cytokine/chemokine expression, (iii) candidate TED-risk gene expression, and (iv) MDSC surface marker expression.

33. The method of claim 32, wherein the genes associated with candidate TED-risk gene expression, comprise any one or more of PROS1, MTR, and PROC APC.

34. The method of claim 26, wherein the any one or more of the genes from Tables 1, 2, and 3, comprise genes associated with any one or more of perianal fistula and perianal disease.

35. The method of claim 34, wherein the genes associated with any one or more of perianal fistula and perianal disease comprise any one or more of CD226, SELP, and PROS1.

36. The method of any one of claims 26-35, wherein differential expression of any one or more of the genes is due to aggregation of CD14+ monocytes with (i) platelets, (ii) megakaryocytes, or (iii) both platelets and megakaryocytes.

37. The method of any one of claims 1-36, wherein the reference expression profile is derived from gene expression levels measured in samples obtained from one or more individuals that:
(a) does not have CD; or
(b) has a subtype of CD that is not characterized by PAF-CD-Mono.

38. A method of determining a Crohn's Disease (CD) subtype status, wherein the status comprises a perianal fistula related CD14+ monocyte subtype (PAF-CD-mono subtype), the method comprising:
detecting the presence of one or more polymorphisms comprising rs763361 or a proxy polymorphism in linkage disequilibrium therewith as determined with an r² of at least 0.85, or a combination thereof, in a biological sample obtained from the subject; and
determining the CD subtype status based on the presence of the one or more polymorphisms,
wherein the presence of the one or more polymorphisms indicates status of PAF-CD-mono subtype.

39. The method of any one of claims 16, 17, and 38, wherein:
(i) the one or more polymorphisms is detected using one or more of a microarray, sequencing, and qPCR; and/or
(ii) the biological sample comprises a blood sample or is purified from a blood sample of the subject.

40. The method of any one of claims 16, 17, 38, and 39, wherein the subject's genotype comprises a rs763361 T variant, optionally wherein the subject is homozygous for the rs763361 T variant.

41. A method for processing or analyzing a biological sample from a subject, comprising:
(a) obtaining the biological sample comprising gene expression products, wherein the subject has or is suspected of having Crohn's Disease (CD);
(b) subjecting the biological sample to an assay by sequencing, array hybridization, and/or nucleic acid amplification to yield a data set including data corresponding to gene expression product levels;
(c) in a programmed computer, inputting said data including said gene expression product levels from (b) to a trained algorithm to generate a classification of said sample as positive or negative for a PAF-CD-mono subtype, wherein the trained algorithm is trained with a plurality of training samples, and wherein said biological sample is independent of said plurality of training samples; and
(d) electronically outputting a report that identifies the classification of the biological sample as positive or negative for the PAF-CD-mono subtype.

42. The method of claim 41, wherein the gene expression products comprise any one or more of CD226, SELP, TNF, CCL4, CXCL3, CCL3, SELPLG, PROS1, PROC, MTR, CD155, and CXCL8.

43. The method of claim 42, wherein the gene expression products comprise (i) SELP, (ii) CD226, or (iii) both SELP and CD226.

44. The method of claim 41, wherein the gene expression products comprise any one or more of CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF.

45. The method of claim 41, wherein the gene expression products comprise any one or more genes from Tables 1, 2, and 3.

46. The method of claim 41, wherein the gene expression products comprise any one or more genes of cell type cluster PAF-CD-mono.

47. A system comprising a kit for determining a Crohn's Disease (CD) subtype status in a subject having CD, the system comprising:
one or more detection reagents comprising nucleic acids configured to hybridize to (i) any one or more genes CD226, SELP, TNF, CCL4, CXCL3, CCL3, SELPLG, PROS1, PROC, MTR, CD155, CXCL8; (ii) CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF; and/or (iii) one or more polymorphisms comprising rs763361 or a proxy polymorphism in linkage disequilibrium therewith as determined with an r² of at least 0.85, or a combination thereof.

48. A system comprising a kit for determining a Crohn's Disease (CD) subtype status in a subject having CD, the system comprising:
one or more detection reagents comprising nucleic acids configured to hybridize to any one or more genes in Tables 1, 2, and 3.

49. A system comprising a kit for determining a Crohn's Disease (CD) subtype status in a subject having CD, the system comprising:
one or more detection reagents comprising nucleic acids configured to hybridize to any one or more genes of cell type cluster PAF-CD-mono.

50. The system of any of claims 47-49, wherein the kit comprises reagents for use in a qPCR reaction.

51. The system of any of claims 47-49, wherein the one or more detection reagents comprises one or more primers or probe.

52. The system of claim 51, wherein the probe comprises a quencher.

53. The system of claims 51 or 52, wherein the probe comprises a detectable label.

54. The system claim 47, wherein the kit further comprises a chip comprising a solid substrate functionalized with oligonucleotides that hybridize to at least a portion of a sequence of the genes (i) CD226, SELP, TNF, CCL4, CXCL3, CCL3, SELPLG, PROS1, PROC, MTR, CD155, CXCL8; and/or (ii) CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF.

55. The system of claim 54, wherein the oligonucleotides that hybridize to the at least a portion of the sequence of the genes comprise the genes of (i) SELP, (ii) CD226, or (iii) both SELP and CD226.

56. The system of claim 47, wherein the kit further comprises a chip comprising a solid substrate functionalized with oligonucleotides that hybridizes to at least a portion of a sequence of the any one or more genes in Tables 1, 2, and 3.

57. The system of claim 49, wherein the kit further comprises a chip comprising a solid substrate functionalized with oligonucleotides that hybridize to at least a portion of a sequence of the any one or more genes of cell type cluster PAF-CD-mono.

58. A computer-implemented platform for determining a Crohn's Disease (CD) subtype status in a subject having CD, wherein the status comprises identifying a perianal fistula related CD14+ monocyte subtype (PAF-CD-mono subtype), the computer-implemented platform comprising:
one or more processors collectively or individually programmed to implement a method comprising:
(a) analyzing gene expression data of the subject to detect a level of expression of any one or more genes CD226, SELP, TNF, CCL4, CXCL3, CCL3, SELPLG, PROS1, PROC, MTR, CD155, and CXCL8, to produce an expression profile of the subject; and
(b) determining the CD subtype status of the subject based upon the expression profile,
wherein differential expression of the one or more genes as compared to a reference expression profile indicates that the CD subtype status of the subject comprises a PAF-CD-mono subtype; and
a database for storing the gene expression data of the subject and/or the expression profile.

59. The computer-implemented platform of claim 58, wherein analyzing the gene expression data of the subject to detect the level of expression of any one or more of the genes comprises detecting the level of expression of (i) SELP, (ii) CD226, or (iii) both SELP and CD226.

60. A computer-implemented platform for determining a Crohn's Disease (CD) subtype status in a subject having CD, wherein the status comprises identifying a perianal fistula related CD14+ monocyte subtype (PAF-CD-mono subtype), the computer-implemented platform comprising:
one or more processors collectively or individually programmed to implement a method comprising:
(a) analyzing gene expression data of the subject to detect a level of expression of any one or more genes in Tables 1, 2, and 3, to produce an expression profile of the subject; and
(b) determining the CD subtype status of the subject based upon the expression profile, wherein differential expression of the one or more genes as compared to a reference expression profile indicates that the CD subtype status of the subject comprises a PAF-CD-Mono subtype; and
a database for storing the gene expression data of the subject and/or the expression profile.

61. The computer-implemented platform of claim 60, wherein the any one or more genes in Tables 1, 2, and 3 comprise (i) SELP, (ii) CD226, or (iii) both SELP and CD226.

62. A computer-implemented platform for determining a Crohn's Disease (CD) subtype status in a subject having CD, wherein the status comprises identifying a perianal fistula related CD14+ monocyte subtype (PAF-CD-mono subtype), the computer-implemented platform comprising:
one or more processors collectively or individually programmed to implement a method comprising:
(a) analyzing gene expression data of the subject to detect a level of expression of any one or more genes of cell type cluster PAF-CD-mono, to produce an expression profile of the subject; and
(b) determining the CD subtype status of the subject based upon the expression profile, wherein differential expression of the one or more genes as compared to a reference expression profile indicates that the CD subtype status of the subject comprises a PAF-CD-mono subtype; and
a database for storing the gene expression data of the subject and/or the expression profile.

63. The computer-implemented platform of claim 62, wherein any one or more of the genes of the cell type cluster PAF-CD-mono comprise (i) SELP, (ii) CD226, or (iii) both SELP and CD226.

64. A computer-implemented platform for determining a Crohn's Disease (CD) subtype status in a subject having CD, wherein the status comprises identifying a perianal fistula related CD14+ monocyte subtype (PAF-CD-mono subtype), the computer-implemented platform comprising:
one or more processors collectively or individually programmed to implement a method comprising:
(a) analyzing gene expression data of the subject to detect a level of expression of any one or more genes CARD9, NOD2, TNFRSF1B, CD93, SELL, ITGAL, ITGAM, IGTB2, TLR1, TLR5, TRLR6, TRAF3, TLR8, TLR4, MYD88, IRAK3, and VWF, to produce an expression profile of the subject; and
(b) determining the CD subtype status of the subject based upon the expression profile, wherein differential expression of the one or more genes as compared to a reference expression profile indicates that the CD subtype status of the subject comprises a PAF-CD-mono subtype; and
a database for storing the gene expression data of the subject and/or the expression profile.

65. The computer implemented platform of any of claims 58-64, further comprising a device for detecting the expression of any one or more genes in a biological sample obtained from the subject.

66. The computer implemented platform of claim 65, wherein the device comprises a microarray, a sequencer, or a qPCR machine.

67. The computer implemented platform of any of claims 58-66, wherein the expression profile is predictive of the PAF-CD-mono subtype with an accuracy of at least 70%, 80%, 90%, or 100%.

68. The computer implemented platform of any of claims 58-66, wherein the expression profile is predictive of the PAF-CD-mono subtype with an area under the curve (AUC) of at least about 0.70, 0.80, 0.90, or 1.0.

69. The computer implemented platform of any of claims 58-66, wherein the expression profile is predictive of PAF-CD-mono subtype with a negative predictive value (NPL) of at least 70%, 80%, 90%, or 100%.

70. The computer implemented platform of any of claims 58-66, wherein the expression profile is predictive of PAF-CD-mono subtype with a positive predictive value (PPV) of at least 70%, 80%, 90%, or 100%.

71. The computer implemented platform of any of claims 58-66, wherein the CD-MNP subtype is characterized by a PAF-CD-mono transcriptomic signature.

72. The computer implemented platform of claim 71, wherein the PAF-CD-mono transcriptomic signature is associated with perianal disease or perianal fistula.

73. The computer implemented platform of any one of claims 58-72, wherein the reference expression profile is derived from gene expression levels measured in samples obtained from one or more individuals that:
(a) does not have CD; or
(b) has a subtype of CD that is not characterized by PAF-CD-Mono.

74. The computer implemented platform of any one of claims 58-73, wherein the differential expression of the one or more genes comprises reduced expression in any one or more of the genes.

75. The computer implemented platform of any one of claims 58-74, wherein the differential expression of the one or more genes comprises increased expression in any one or more of the genes.

76. A method of treating a Crohn's Disease (CD) subtype status, wherein the status comprises a perianal fistula related CD14+ monocyte subtype (PAF-CD-mono subtype), the method comprising:
detecting expression of (i) SELP, (ii) CD226, or (iii) both SELP and CD226;
determining the CD subtype status based on detecting expression of SELP, CD226, or both SELP and CD226,
wherein differential expression of SELP, CD226, or both SELP and CD226, as compared to a reference expression profile indicates status of PAF-CD-mono subtype; and
administering a therapeutically effective amount of a therapeutic agent for treating CD.

77. The method of claim 76, wherein differential expression of SELP, CD226, or both SELP and CD226 comprises reduced expression of SELP, CD226, or both SELP and CD226.

78. The method of claim 76 or 77, wherein the therapeutic agent comprises an anti-TL1A antibody.

79. The method of claim 76 or 77, wherein the therapeutic agent comprises a steroid.

80. The method of claim 76 or 77, wherein the therapeutic agent comprises an immunosuppressant.

81. The method of any of claims 76-80, wherein differential expression of SELP, CD226, or both SELP and CD226 is due to aggregation of CD14+ monocytes with (i) platelets, (ii) megakaryocytes, or (iii) both platelets and megakaryocytes.

82. The method of any of claims 76-81, wherein SELP, CD226, or both SELP and CD226 are associated with any one or more of perianal fistula and perianal disease.

83. A method of treating moderate to severe Crohn's Disease (CD) in a subject, the method comprising:
administering a therapeutically effective amount of a therapeutic agent for treatment of the CD, provided the subject is determined to have a perianal fistula related CD14+ monocyte subtype (PAF-CD-mono subtype) based, at least in part, on differential expression of (i) SELP, (ii) CD226, or (iii) both SELP and CD226 in a biological sample obtained from the subject, relative to a reference expression profile.

84. The method of claim 83, wherein differential expression of SELP, CD226, or both SELP and CD226 comprises reduced expression of SELP, CD226, or both SELP and CD226.

85. The method of claim 83 or 84, wherein the PAF-CD-mono subtype is associated with any one or more of perianal disease or perianal fistula.

86. The method of any of claims 83-85, wherein the therapeutic agent comprises an anti-TL1A antibody.

87. The method of any of claims 83-85, wherein the therapeutic agent comprises a steroid.

88. The method of any of claims 83-85, wherein the therapeutic agent comprises an immunosuppressant.

89. The method of any of claims 83-88, wherein differential expression of SELP, CD226, or both SELP and CD226 is due to aggregation of CD14+ monocytes with (i) platelets, (ii) megakaryocytes, or (iii) both platelets and megakaryocytes.

90. The method of any one of claims 83-89, wherein differential expression of SELP, CD226, or both SELP and CD226 is associated with any one or more of perianal fistula and perianal disease.

91. A method of determining a Crohn's Disease (CD) subtype status, wherein the status comprises a perianal fistula related CD14+ monocyte subtype (PAF-CD-mono subtype), the method comprising:
detecting expression of (i) SELP, (ii) CD226, or (iii) both SELP and CD226; and
determining the CD subtype status based on the expression of (i), (ii), or (iii),
wherein differential expression in the any one or more of SELP, CD226, or both SELP and CD226 as compared to a reference expression profile indicates status of PAF-CD-mono subtype.

92. The method of claim 91, wherein differential expression of SELP, CD226, or both SELP and CD226 comprises reduced expression of SELP, CD226, or both SELP and CD226.

93. The method of claim 91 or 92, wherein differential expression of SELP, CD226, or both SELP and CD226 is due to aggregation of CD14+ monocytes with (i) platelets, (ii) megakaryocytes, or (iii) both platelets and megakaryocytes.

94. The method of any of claims 91-93, wherein the PAF-CD-mono subtype is associated with any one or more of perianal disease or perianal fistula.

95. The method of any of claims 76-94, wherein the reference expression profile is derived from gene expression levels measured in samples obtained from one or more individuals that:
(a) does not have CD; or
(b) has a subtype of CD that is not characterized by PAF-CD-mono.
